(19) United States
(12) Patent Application Publication
(10) Pub. No.: US 2007/0105914 A1
(43) Pub. Date: May 10, 2007

(54) Substituted sulfonamides

(76) Inventors: Helen M. Armstrong, Westfield, NJ (US); Linda L. Chang, Wayne, NJ (US); Ravindra N. Guthikonda, Edison, NJ (US); William K. Hagmann, Westfield, NJ (US); Linus S. Lin, Westfield, NJ (US); Shrenik K. Shah, Metuchen, NJ (US)

Correspondence Address: MERCK AND CO., INC, P O BOX 2000, RAHWAY, NJ, 07065-0907, US

(21) Appl. No.: 10/571,842

(22) PCT Filed: Sep. 14, 2004
(86) PCT No.: PCT/US04/30122

§ 371 Date: Mar. 15, 2006

Related U.S. Application Data
(60) Provisional application No. 60/504,377, filed on Sep. 18, 2003.

(51) Int. Cl. A61K 31/44; A61K 31/277; A61K 31/18

(52) U.S. Cl. 514/357; 564/86; 514/602; 514/520; 558/408; 546/329

(57) ABSTRACT

The substituted sulfonamides of the invention are antagonists and/or inverse agonists of the Cannabinoid-1 (CB1) receptor and are useful in the treatment, prevention and suppression of diseases mediated by the CB1 receptor. The compounds of the present invention are useful as centrally acting drugs in the treatment of psychosis, memory deficits, cognitive disorders, migraine, neuropathy, neuro-inflammatory disorders including multiple sclerosis and Guillain-Barre syndrome and the inflammatory sequelae of viral encephalitis, cerebral vascular accidents, and head trauma, anxiety disorders, stress, epilepsy, Parkinson's disease, movement disorders, and schizophrenia The compounds are also useful for the treatment of substance abuse disorders, the treatment of obesity or eating disorders, as well as the treatment of asthma, constipation, chronic intestinal pseudo-obstruction, and cirrhosis of the liver.

### BACKGROUND OF THE INVENTION

Marijuana (Cannabis sativa L.) and its derivatives have been used for centuries for medicinal and recreational purposes. A major active ingredient in marijuana and hashish has been determined to be Δ⁹-tetrahydrocannabinol (Δ⁹-THC). Detailed research has revealed that the biological action of Δ⁹-THC and other members of the cannabinoid family occurs through two G-protein coupled receptors termed CB1 and CB2. The CB1 receptor is primarily found in the central and peripheral nervous systems and to a lesser extent in several peripheral organs. The CB2 receptor is found primarily in lymphoid tissues and cells. Three endogenous ligands for the cannabinoid receptors derived from arachidonic acid have been identified (anandamide, 2-arachidonoyl glycerol, and 2-arachidonyl glycerol ether). Each is an agonist with activities similar to Δ⁹-THC, including sedation, hypotherrnia, intestinal immobility, antinociception, analgesia, catalepsy, anti-emesis, and appetite stimulation.

There is at least one CB1 modulator characterized as an inverse agonist or an antagonist, N-(1-piperidinyl)-5-(4-chlorophenyl)-1-(2,4-dichlorophenyl)-4-methylpyrazole-3-carboxamide (SR141716A), in clinical trials for treatment of eating disorders at this time. There still remains a need for potent low molecular weight CB1 modulators that have pharmacokinetic and pharmacodynamic properties suitable for use as human pharmaceuticals.

U.S. Pat. Nos. 5,624,941, 6,028,084, and 6,509,367, PCT Publications WO98/43636 and WO98/43635, and EP-658546 disclose substituted pyrazoles having activity against the cannabinoid receptors. PCT Publications WO98/31227 and WO98/41519 also disclose substituted pyrazoles having activity against the cannabinoid receptors. PCT Publications WO98/37061, WO00/10967, and WO00/10968 disclose diaryl ether sulfonamides having activity against the cannabinoid receptors. PCT Publications WO97/29079 and WO99/02499 disclose alkoxy-isoindolones and alkoxy-quinolones as having activity against the cannabinoid receptors. U.S. Pat. No. 5,532,237 discloses N-benzoyl-indole derivatives having activity against the cannabinoid receptors. US Patents U.S. Pat. Nos. 4,973,587, 5,013,837, 5,081,122, and 5,112,820, 5,292,736 disclose aminoalkylindole derivatives as having activity against the cannabinoid receptors. PCT publication WO 01/58869 discloses pyrazoles, pyrroles and imidazole cannabinoid receptor modulators useful for treating respiratory and non-respiratory leukocyte activation-associated disorders. United States patents U.S. Pat. No. 6,355,631, and U.S. Pat. No. 6,479,479 and PCT publications WO 01/64632, 01/64633, and 01/64634 are directed to azetidine derivatives as cannabinoid antagonists. Other cannabinoid receptor modulating compounds are disclosed in WO 01/70700, WO 02/076949; WO 03/026647; WO 03/026648; WO 03/027069; WO 03/027076; and WO 03/027114.

### SUMMARY OF THE INVENTION

The present invention is concerned with substituted sulfonamide derivatives of general formula I: 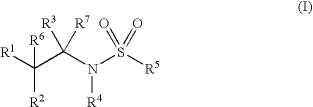
stereoisomers and pharmaceutically acceptable salts thereof which are antagonists and/or inverse agonists of the Cannabinoid-1 (CB1) receptor and are useful in the treatment, prevention or suppression of diseases mediated by the Cannabinoid-1 (CB1) receptor. The invention is concerned with the use of these novel compounds to selectively antagonize the Cannabinoid-1 (CB1) receptor. As such, compounds of the present invention are useful as centrally acting drugs in the treatment of psychosis, memory deficits, cognitive disorders, migraine, neuropathy, neuro-inflammatory disorders including multiple sclerosis and Guillain-Barre syndrome and the inflammatory sequelae of viral encephalitis, cerebral vascular accidents, and head trauma, anxiety disorders, stress, epilepsy, Parkinson's disease, movement disorders, and schizophrenia. The compounds are also useful for the treatment of substance abuse disorders, such as for example, those relating to opiates, alcohol, marijuana, and nicotine, including smoking cessation. The compounds are also useful for the treatment of obesity or eating disorders associated with excessive food intake and complications associated therewith, including left ventricular hypertrophy. The compounds are also useful for the treatment of constipation and chronic intestinal pseudo-obstruction. The compounds are also useful for the treatment of cirrhosis of the liver. The compounds are also useful for the treatment of asthma.

The present invention is also concerned with treatment of these conditions, and the use of compounds of the present invention for manufacture of a medicament useful in treating these conditions. The present invention is also concerned with treatment of these conditions through a combination of compounds of formula I and other currently available pharmaceuticals.

The invention is also concerned with pharmaceutical formulations comprising a compound of structural formula I as an active ingredient.

The invention is further concerned with processes for preparing the compounds of this invention.

### DETAILED DESCRIPTION OF THE INVENTION

The compounds of the present invention are represented by structural formula I: 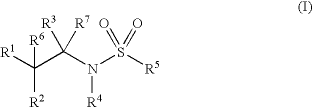
or a pharmaceutically acceptable salt or stereoisomer thereof, wherein:
R¹ is chosen from:
   (1) C₁₋₁₀alkyl,
   (2) C₃₋₁₀cycloalkyl-C₀₋₄alkyl,
   (3) cycloheteroalkyl-C₀₋₄alkyl,
   (4) aryl-C₀₋₄alkyl,
   (5) heteroaryl-C₁₋₄alkyl,
   (6) —OR^{d},
   (7) —SR^{d},
   (8) —(C═O)_{z}NR^{c}R^{d},
   (9) —NR^{c}C(O)R^{d}, and
   (10) —CO₂R^{d},
   wherein each alkyl is optionally substituted with one to four substituents independently chosen from R^{a}, and each cycloalkyl, and cycloheteroalkyl, aryl, and heteroaryl are optionally substituted with one to four substituents independently chosen from R^{b};
R² is chosen from:
   (1) C₁₋₁₀alkyl,
   (2) C₃₋₁₀cycloalkyl-C₀₋₄akyl,
   (3) cycloheteroalkyl-C₀₋₄alkyl,
   (4) aryl-C₀₋₄alkyl, and
   (5) heteroaryl-C₀₋₄alkyl,
   wherein each alkyl is optionally substituted with one to four substituents independently chosen from R^{a}, and each cycloalkyl, cycloheteroalkyl, aryl and heteroaryl is optionally substituted with one to four substituents independently chosen from R^{b};
R³ and R⁷ are each independently chosen from:
   (1) hydrogen,
   (2) C₃₋₁₀cycloalkyl-C₀₋₄alkyl,
   (3) cycloheteroalkyl-C₀₋₄alkyl,
   (4) aryl-C₀₋₄alkyl,
   (5) heteroaryl-C₀₋₄alkyl, and
   (6) C₁₋₄alkyl,
   wherein each alkyl is optionally substituted with one to four substituents independently chosen from R^{a}, and each cycloalkyl, cycloheteroalkyl, aryl and heteroaryl is optionally substituted with one to four substituents independently chosen from R^{b};
R⁴ is chosen from:
   (1) hydrogen, and
   (2) C₁₋₄alkyl,
   wherein each alkyl is optionally substituted with one to four substituents independently chosen from R^{a};
R⁵ is chosen from:
   (1) C₁₋₄alkyl,
   (2) C₂₋₁₀alkenyl,
   (3) C₂₋₁₀alkynyl,
   (4) C₃₋₁₀cycloalkyl-C₀₋₄alkyl,
   (5) cycloheteroalkyl-C₀₋₄alkyl,
   (6) aryl-C₀₋₄alkyl,
   (7) heteroaryl-C₁₋₄alkyl,
   (8) —NR^{c}R^{d}, and
   (9) —NR^{c}C(O)R^{d},
   wherein alkyl, alkenyl, and alkynyl are optionally substituted with one to four substituents independently chosen from R^{a} and cycloalkyl, cycloheteroalkyl, aryl and heteroaryl are optionally substituted with one to four substituents independently chosen from R^{b};
R⁶ is chosen from:
   (1) hydrogen,
   (2) hydroxyl,
   (3) C₁₋₄alkyl,
   (4) halogen, and
   (5) cyano,
provided that when R¹ is —OR^{d}, —SR^{d}, or —NR^{c}C(O)R^{d}, then R⁶ is chosen from hydrogen and C₁₋₄alkyl; each R^{a} is independently chosen from:
   (1) —OR^{d},
   (2) —NR^{c}S(O)ₘR^{d},
   (3) halogen,
   (4) —SR^{d},
   (5) —S(O)ₘNR^{c}R^{d},
   (6) —(C═O)_{z}NR^{c}R^{d},
   (7) —C(O)R^{d},
   (8) —CO₂R^{d},
   (9) —CN,
   (10) —NR^{c}C(O)R^{d},
   (11) —NR^{c}C(O)OR^{d},
   (12) —NR^{c}C(O)NR^{c}R^{d},
   (13) —CF₃,
   (14) —OCF₃, and
   (15) cycloheteroalkyl;
each R^{b} is independently chosen from:
   (1) R^{a},
   (2) C₁₋₁₀alkyl,
   (3) oxo,
   (4) arylC₀₋₄alkyl, and
   (5) heteroarylC₀₋₄alkyl,
R^{c} and R^{d} are independently chosen from:
   (1) hydrogen,
   (2) C₁₋₁₀alkyl,
   (3) C₂₋₁₀alkenyl,
   (4) cycloalkyl-C₀₋₁₀alkyl;
   (5) cycloheteroalkyl-C₀₋₁₀alkyl;
   (6) aryl-C₀₋₁₀alkyl, and
   (7) heteroaryl-C₁₋₁₀alkyl, wherein:
R^{c} and R^{d} together with the atom(s) to which they are attached optionally form a heterocyclic ring of 4 to 7 members containing 0-2 additional heteroatoms independently chosen from oxygen, sulfur and N—R_{g}, and
each R^{c} and R^{d} can be optionally substituted with one to three substituents chosen from R^{h}; each R_{g} is independently chosen from
   (1) C₁₋₁₀alkyl,
   (2) —C(O)R^{c},
   (3) —C(O)H,
   (4) —C(O)C₁₋₁₀alkyl,
   (5) —C(O)C₂₋₁₀alkenyl,
   (6) —C(O)C₀₋₁₀alkylcycloalkyl,
   (7) —C(O)C₀₋₁₀alkylcycloheteroalkyl,
   (8) —C(O)C₀₋₁₀alkylaryl, and
   (9) —C(O)C₀₋₁₀alkyl heteroaryl;
each R^{h} is independently chosen from:
   (1) halogen,
   (2) C₁₋₁₀alkyl,
   (3) —O—C₁₋₄alkyl,
   (4) —S—C₁₋₄alkyl,
   (5) —CN,
   (6) —NO₂,
   (7) —CF₃, and
   (8) —OCF₃;
m is chosen from 1 and 2; and
z is chosen from 0 and 1.

In one embodiment of the invention, R⁵ is chosen from: C₁₋₁₀alkyl, aryl-C₀₋₄alkyl, and heteroaryl-C₁₋₄alkyl, wherein alkyl is optionally substituted with one to four substituents independently chosen from R^{a} and aryl and heteroaryl are optionally substituted with one to four substituents independently chosen from R^{b}.

In another embodiment, R³ and R⁷ are each independently chosen from: hydrogen, aryl-C₀₋₄alkyl, and C₁₋₄alkyl, wherein each alkyl is optionally substituted with one to four substituents independently chosen from R^{a}, and each cycloalkyl, cycloheteroalkyl, aryl and heteroaryl is optionally substituted with one to four substituents independently chosen from R^{b}.

In an embodiment, the compounds of the invention are chosen from those wherein:
R¹ is chosen from:
   (1) C₁₋₁₀alkyl,
   (2) C₃₋₁₀cycloalkyl-C₀₋₄alkyl,
   (3) cycloheteroalkyl-C₀₋₄alkyl,
   (4) aryl-C₀₋₄alkyl,
   (5) heteroaryl-C₁₋₄alkyl,
   (6) —OR^{d},
   (7) —SR^{d},
   (8) —(C═O)_{z}NR^{c}R^{d},
   (9) —NR^{c}C(O)R^{d}, and
   (10) —O₂R^{d},
   wherein each alkyl is optionally substituted with one to four substituents independently chosen from R^{a}, and each cycloalkyl, and cycloheteroalkyl, aryl, and heteroaryl are optionally substituted with one to four substituents independently chosen from R^{b};
R² is R^{2′}—Y—;
Y is C₀₋₄alkyl optionally substituted with one to four substituents independently chosen from R^{a};
R^{2′} is chosen from: aryl and heteroaryl, wherein each aryl and heteroaryl is optionally substituted with one to four substituents independently chosen from R^{b};
R³ and R⁷ are each independently chosen from:
   (1) hydrogen,
   (2) aryl-C₀₋₄alkyl, and
   (3) C₁₋₄alkyl,
   wherein each alkyl is optionally substituted with one to four substituents independently chosen from R^{a}, and each cycloalkyl, cycloheteroalkyl, aryl and heteroaryl is optionally substituted with one to four substituents independently chosen from R^{b};
R⁴ is chosen from:
   (1) hydrogen, and
   (2) C₁₋₄alkyl,
   wherein each alkyl is optionally substituted with one to four substituents independently chosen from R^{a};
R⁵ is chosen from:
   (1) C₁₋₁₀alkyl,
   (2) C₂₋₁₀alkenyl,
   (3) C₂₋₁₀alkynyl,
   (4) C₃₋₁₀cycloalkyl-C₀₋₄alkyl,
   (5) cycloheteroalkyl-C₀₋₄alkyl,
   (6) aryl-C₀₋₄alkyl,
   (7) heteroaryl-C₁₋₄alkyl,
   (8) —NR^{c}R^{d}, and
   (9) —NR^{c}C(O)R^{d},
   wherein alkyl, alkenyl, and alkynyl, are optionally substituted with one to four substituents independently chosen from R^{a} and cycloalkyl, cycloheteroalkyl, aryl and heteroaryl are optionally substituted with one to four substituents independently chosen from R^{b};
R⁶ is chosen from:
   (1) hydrogen,
   (2) hydroxyl,
   (3) C₁₋₄alkyl,
   (4) halogen, and
   (5) cyano,
provided that when R¹ is —OR^{d}, —SR^{d}, or —NR^{c}C(O)R^{d}, then R⁶ is chosen from hydrogen and C₁₋₄alkyl; each R^{a} is independently chosen from:
   (1) —OR^{d},
   (2) —NR^{c}S(O)ₘR^{d},
   (3) halogen,
   (4) —SR^{d},
   (5) —S(O)ₘNR^{c}R^{d},
   (6) —(C═O)_{z}NR^{c}R^{d},
   (7) —C(O)R^{d},
   (8) —CO₂R^{d},
   (9) —CN,
   (10) —NR^{c}C(O)R^{d},
   (11) —NR^{c}C(O)OR^{d},
   (12) —NR^{c}C(O)NR^{c}R^{d},
   (13) —CF₃,
   (14) —OCF₃, and
   (15) cycloheteroalkyl;
each R^{b} is independently chosen from:
   (1) R^{a},
   (2) C₁₋₁₀alkyl,
   (3) oxo,
   (4) arylC₀₋₄alkyl, and
   (5) heteroarylC₀₋₄alkyl;
R^{c} and R^{d} are independently chosen from:
   (1) hydrogen,
   (2) C₁₋₁₀alkyl,
   (3) C₂₋₁₀alkenyl,
   (4) cycloalkyl-C₀₋₁₀alkyl;
   (5) cycloheteroalkyl-C₀₋₁₀alkyl;
   (6) aryl-C₀₋₁₀alkyl, and
   (7) heteroaryl-C₁₋₁₀alkyl, or
R^{c} and R^{d} together with the atom(s) to which they are attached form a heterocyclic ring of 4 to 7 members containing 0-2 additional heteroatoms independently chosen from oxygen, sulfur and N—R_{g},
each R^{c} and R^{d} may be unsubstituted or substituted with one to three substituents chosen from R^{h}; each R_{g} is independently chosen from
   (1) C₁₋₁₀alkyl, and
   (2) —C(O)R^{c};
each R^{h} is independently chosen from:
   (1) halogen,
   (2) C₁₋₁₀alkyl,
   (3) —O—C₁₋₄alkyl,
   (4) —S—C₁₋₄alkyl,
   (5) —CN,
   (6) —NO₂,
   (7) —CF₃, and
   (8) —OCF₃;
m is chosen from 1 and 2; and
z is chosen from 0 and 1.

In another embodiment, R^{2′} is chosen from: 2,3-dihydro-1H-indolyl, 3,4-dihydroquinolinyl, phenyl, benzyl, and pyridinyl, and R^{2′} is optionally substituted with one to four substituents independently chosen from R^{b}.

In yet another embodiment, Y is —CH₂—.

Compounds of the present invention may also be chosen from compounds of structural formula II 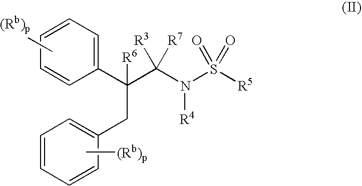
or a pharmaceutically acceptable salt or stereoisomer thereof, wherein:
R³ and R⁷ are each independently chosen from:
   (1) hydrogen,
   (2) C₃₋₁₀cycloalkyl-C₀₋₄alkyl,
   (3) cycloheteroalkyl-C₀₋₄alkyl,
   (4) aryl-C₀₋₄alkyl, and
   (5) heteroaryl-C₀₋₄alkyl, and
   (6) C₁₋₄alkyl,
   wherein each alkyl is optionally substituted with one to four substituents independently chosen from R^{a}, and each cycloalkyl, cycloheteroalkyl, aryl and heteroaryl is optionally substituted with one to four substituents independently chosen from R^{b};
R⁴ is chosen from:
   (1) hydrogen, and
   (2) C₁₋₄alkyl,
   wherein each alkyl is optionally substituted with one to four substituents independently chosen from R^{a};
R⁵ is chosen from:
   (1) C₁₋₁₀alkyl,
   (2) C₂₋₁₀alkenyl,
   (3) C₂₋₁₀alkynyl,
   (4) C₃₋₁₀cycloalkyl-C₀₋₄alkyl,
   (5) cycloheteroalkyl-C₀₋₄alkyl,
   (6) aryl-C₀₋₄alkyl,
   (7) heteroaryl-C₁₋₄alkyl,
   (8) —NR^{c}R^{d}, and
   (9) —NR^{c}C(O)R^{d},
   wherein alkyl, alkenyl, and alkynyl, are optionally substituted with one to four substituents independently chosen from R^{a} and cycloalkyl, cycloheteroalkyl, aryl and heteroaryl are optionally substituted with one to four substituents independently chosen from R^{b};
R⁶ is chosen from:
   (1) hydrogen,
   (2) hydroxyl,
   (3) C₁₋₄alkyl,
   (4) halogen, and
   (5) cyano,
each R^{a} is independently chosen from:
   (1) —OR^{d},
   (2) —NR^{c}S(O)ₘR^{d},
   (3) halogen,
   (4) —SR^{d},
   (5) —S(O)ₘNR^{c}R^{d},
   (6) —C(═O)_{z}NR^{c}R^{d},
   (7) —C(O)R^{d},
   (8) —CO₂Rd,
   (9) —CN,]—NR^{c}C(O)R^{d},
   (10) —NR^{c}C(O)OR^{d},
   (11) —NR^{c}C(O)NR^{c}R^{d},
   (12) —CF₃,
   (13) —OCF₃, and
   (14) cycloheteroalkyl;
each R^{b} is independently chosen from:
   (1) R^{a},
   (2) C₁₋₁₀alkyl,
   (3) oxo,
   (4) arylC₀₋₄alkyl, and
   (5) heteroarylC₀₋₄alkyl,
R^{c} and R^{d} are independently chosen from:
   (1) hydrogen,
   (2) C₁₋₁₀alkyl,
   (3) C₂₋₁₀alkenyl,
   (4) cycloalkyl-C₀₋₁₀alkyl;
   (5) cycloheteroalkyl-C₀₋₁₀alkyl;
   (6) aryl-C₀₋₁₀alkyl, and
   (7) heteroaryl-C₁₋₁₀alkyl, or
R^{c} and R^{d} together with the atom(s) to which they are attached form a heterocyclic ring of 4 to 7 members containing 0-2 additional heteroatoms independently chosen from oxygen, sulfur and N—R_{g},
each R^{c} and R^{d} may be unsubstituted or substituted with one to three substituents chosen from R^{h}; each R_{g} is independently chosen from
   (1) C₁₋₁₀alkyl, and
   (2) —C(O)R^{c};
each R^{h} is independently chosen from:
   (1) halogen,
   (2) C₁₋₁₀alkyl,
   (3) —O—C₁₋₄alkyl,
   (4) —S—C₁₋₄alkyl,
   (5) —CN,
   (6) —NO₂,
   (7) —CF₃, and
   (8) —OCF₃;
m is chosen from 1 and 2;
p is 0, 1, 2, 3, or 4; and
z is chosen from 0 and 1.

In one embodiment of the invention, R³ and R⁷ are each independently chosen from: hydrogen, aryl-C₀₋₄alkyl, and C₁₋₄alkyl, wherein each alkyl is optionally substituted with one to four substituents independently chosen from R^{a}, and aryl is optionally substituted with one to four substituents independently chosen from R^{b}.

In another embodiment, R⁵ is chosen from: C₁₋₁₀alkyl, and aryl-C₀₋₄alkyl, wherein alkyl is optionally substituted with one to four substituents independently chosen from R^{a} and aryl is optionally substituted with one to four substituents independently chosen from R^{b}.

In yet another embodiment of the invention, R⁶ is chosen from hydrogen, hydroxyl, and halogen.

“Alkyl”, as well as other groups having the prefix “alk”, such as alkoxy, alkanoyl, means carbon chains which may be linear or branched or combinations thereof. Examples of alkyl groups include methyl, ethyl, n-propyl, isopropyl, butyl, isobutyl, sec- and tert-butyl, pentyl, hexyl, heptyl, octyl, nonyl, and the like. The term “C₀ alkyl” (as in “C₀₋₈ alkylaryl”) shall refer to the absence of an alkyl group.

The term “alkenyl” shall mean straight or branched chain alkenes of two to ten total carbon atoms, or any number within this range. Examples of alkenyl include vinyl, allyl, isopropenyl, pentenyl, hexenyl, heptenyl, 1-propenyl, 2-butenyl, 2-methyl-2-butenyl, and the like.

The term “alkynyl” refers to a hydrocarbon radical straight, branched or cyclic, containing from 2 to 10 carbon atoms and at least one carbon to carbon triple bond. Up to three carbon-carbon triple bonds can be present. Thus, “C₂₋₆ alkynyl” means an alkynyl radical having from 2 to 6 carbon atoms. Alkynyl groups include ethynyl, propynyl, butynyl, 3-methylbutynyl and so on. The straight, branched or cyclic portion of the alkynyl group can contain triple bonds and can be substituted if a substituted alkynyl group is indicated. Examples of alkynyl include ethynyl, propargyl, 3-methyl-1-pentynyl, 2-heptynyl and the like.

“Cycloalkyl” as used herein is intended to include non-aromatic cyclic hydrocarbon groups, having the specified number of carbon atoms, which may or may not be bridged or structurally constrained. Examples of such cycloalkyls include, but are not limited to, cyclopropyl, cyclobutyl, cyclopentyl, cyclohexyl, adamantyl, cyclooctyl, cycloheptyl, tetrahydro-naphthalene, methylenecylohexyl, and the like. As used herein, examples of “C₃-C₁₀ cycloalkyl” can include, but are not limited to:

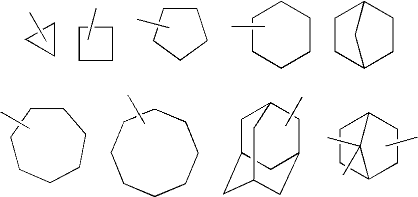

As used herein, “aryl” is intended to mean any stable monocyclic or bicyclic carbon ring of up to 7 atoms in each ring, wherein at least one ring is aromatic. Examples of such aryl elements include, but are not limited to, phenyl, naphthyl, tetrahydro-naphthyl, indanyl, or biphenyl. In cases where the aryl substituent is bicyclic and one ring is non-aromatic, it is understood that attachment is via the aromatic ring.

“Heteroaryl” means a mono- or bicyclic aromatic ring containing at least one heteroatom selected from N, O, and S, with each ring containing 5 to 6 atoms. Examples of heteroaryl include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrmidyl, pyridazinyl, pyrazinyl, benzoxazolyl, benzothiazolyl, benzimidazolyl, benzofuranyl, benzothiophenyl, furo(2,3-b)pyridyl, quinolyl, indolyl, isoquinolyl, imidazothiazolyl, and the like. The heteroaryl ring may be substituted on one or more carbon or nitrogen atoms

“Cycloheteroalkyl” means mono- or bicyclic or bridged saturated rings containing at least one heteroatom selected from N, S and O, each of said ring having from 3 to 10 atoms in which the point of attachment may be carbon or nitrogen. The term also includes monocyclic heterocycle fused to an aryl or heteroaryl group in which the point of attachment is on the non-aromatic portion. Examples of “cycloheteroalkyl” include pyrrolidinyl, piperidinyl, piperazinyl, dioxanyl, imidazolidinyl, 2,3-dihydrofuro(2,3-b)pyridyl, benzoxazinyl, benzoxazolinyl, 2-H-phthalazinyl, isoindolinyl, benzoxazepinyl, 5,6-dihydroimidazo[2,1-b]thiazolyl, tetrahydrohydroquinolinyl, morpholinyl, tetrahydroisoquinolinyl, dihydroindolyl, and the like. The term also includes partially unsaturated monocyclic rings that are not aromatic, such as 2- or 4-pyridones attached through the nitrogen or N-substituted-(1H,3H)-pyrimidine-2,4-diones (N-substituted uracils). The term also includes bridged rings such as 5-azabicyclo[2.2.1]heptyl, 2,5-diazabicyclo[2.2.1]heptyl, 2-azabicyclo[2.2.1]heptyl, 7-azabicyclo[2.2.1]heptyl, 2,5-diazabicyclo[2.2.2]octyl, 2-azabicyclo[2.2.2]octyl, and 3-azabicyclo[3.2.2]nonyl, and azabicyclo[2.2.1]heptanyl. The cycloheteroalkyl ring may be substituted on the ring carbons and/or the ring nitrogens.

The term “oxy” means an oxygen (O) atom. The term “thio” means a sulfur (S) atom. The term “oxo” means “═O”. The term “carbonyl” means “C═O.”

As appreciated by those of skill in the art, “halo” or “halogen” as used herein is intended to include chloro, fluoro, bromo and iodo.

When any variable (e.g., R¹, R^{d}, etc.) occurs more than one time in any constituent or in formula I, its definition on each occurrence is independent of its definition at every other occurrence. Also, combinations of substituents and/or variables are permissible only if such combinations result in stable compounds.

Under standard nomenclature used throughout this disclosure, the terminal portion of the designated side chain is described first, followed by the adjacent functionality toward the point of attachment. For example, a C₁₋₅ alkylcarbonylamino C₁₋₆ alkyl substituent is equivalent to:

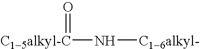

In choosing compounds of the present invention, one of ordinary skill in the art will recognize that the various substituents, i.e. R¹, R², etc., are to be chosen in conformity with well-known principles of chemical structure connectivity and stability.

The term “substituted” shall be deemed to include multiple degrees of substitution by a named substitutent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

Lines drawn into the ring systems from substituents indicate that the indicated bond can be attached to any of the substitutable ring atoms. If the ring system is polycyclic, it is intended that the bond be attached to any of the suitable carbon atoms on the proximal ring only.

It is understood that substituents and substitution patterns on the compounds of the instant invention can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups can be on the same carbon or on different carbons, so long as a stable structure results. The phrase “optionally substituted with one or more substituents” should be taken to be equivalent to the phrase “optionally substituted with at least one substituent” and in such cases one embodiment will have from zero to three substituents.

In one embodiment, R¹ is chosen from: C₁₋₁₀alkyl, C₃₋₁₀cycloalkyl-C₀₋₄alkyl, cycloheteroalkyl-C₀₋₄alkyl, aryl-C₀₋₄alkyl, heteroaryl-C₁₋₄alkyl, OR^{d}, —SR^{d}, —(C═O)_{z}NR^{c}R^{d}, and —CO₂R^{d}, wherein each alkyl is optionally substituted with one to four substituents independently chosen from R^{a}, and each cycloalkyl, and cycloheteroalkyl, aryl, and heteroaryl are optionally substituted with one to four substituents independently chosen from R^{b};

In another embodiment, R¹ is chosen from: C₃₋₁₀cycloalkyl-C₀₋₄alkyl, cycloheteroalkyl-C₀₋₄alkyl, aryl-C₀₋₄alkyl, heteroaryl-C₁₋₄alkyl, —OR^{d}, —S R^{d}, and —CO₂R^{d}, wherein each alkyl is optionally substituted with one to four substituents independently chosen from R^{a}, and each cycloalkyl, and cycloheteroalkyl, aryl, and heteroaryl are optionally substituted with one to four substituents independently chosen from R^{b}.

In another embodiment, R¹ is chosen from cyclopentyl-C₀₋₄alkyl, cyclobutyl-C₀₋₄alkyl, cyclopropyl-C₀₋₄alkyl, piperidinyl-C₀₋₄alkyl, pyridyl-C₀₋₄alkyl, pyrrolidinyl-C₀₋₄alkyl, triazolyl-C₀₋₄alkyl, indolinyl-C₀₋₄alkyl, 7-azaindolyl-C₀₋₄alkyl, benzisoxazolyl-C₀₋₄alkyl, 3,4-dihydroquinolinyl-C₀₋₄alkyl, 1H-1,2,3-benzotriazolyl-C₀₋₄alkyl, thiophenyl-C₀₋₄alkyl, pyridazinyl-C₀₋₄alkyl, pyrimidinyl-C₀₋₄alkyl, phenyl, benzyl, —CO₂(C₀₋₄alkyl ), —CO₂aryl, —OC₀₋₁₀alkylaryl, —OC₀₋₁₀alkylcycloalkyl, —SC₀₋₁₀alkylaryl, —N(C₁₋₁₀alkyl)aryl-C₀₋₄alkyl, —CO₂R wherein each alkyl is optionally substituted with one to four substituents independently chosen from R^{a}, and each cycloalkyl and cycloheteroalkyl is optionally substituted with one to four substituents independently chosen from R^{b}.

In one embodiment of the invention, R² is chosen from: C₁₋₁₀alkyl, cycloheteroalkyl-C₀₋₄alkyl, aryl-C₀₋₄alkyl, and heteroaryl-C₀₋₄alkyl,wherein each alkyl is optionally substituted with one to four substituents independently chosen from R^{a}, and each cycloheteroalkyl, aryl and heteroaryl is optionally substituted with one to four substituents independently chosen from R^{b}.

In a variant of this embodiment, R² is chosen from aryl-C₀₋₄alkyl, optionally substituted with one to four substituents independently chosen from R^{b}.

In another embodiment of the invention, R³ and R⁷ are each independently chosen from: hydrogen, C₃₋₁₀cycloalkyl-C₀₋₄alkyl, aryl-C₀₋₄alkyl, and C₁₋₄alkyl, wherein each alkyl is optionally substituted with one to four substituents independently chosen from R^{a}, and each cycloalkyl, and aryl, is optionally substituted with one to four substituents independently chosen from R^{b}. In one variant of this embodiment, R³ and R⁷ are each independently chosen from: hydrogen and C₁₋₄alkyl optionally substituted with one to four substituents independently chosen from R^{a}.

In one embodiment, R⁴ is hydrogen. In another embodiment, R⁴ is C₁₋₄alkyl optionally substituted with one to four substuents independently chosen from R^{a}.

In one embodiment of the invention, R⁵ is chosen from: C₁₋₁₀alkyl, C₃₋₁₀cycloalkyl-C₀₋₄alkyl, cycloheteroalkyl-C₀₋₄alkyl, aryl-C₀₋₄alkyl; and heteroaryl-C₁₋₄alkyl, wherein alkyl is optionally substituted with one to four substituents independently chosen from R^{a} and cycloalkyl, cycloheteroalkyl, aryl and heteroaryl are optionally substituted with one to four substituents independently chosen from R^{b}.

In another embodiment, R⁵ is chosen from: C₁₋₁₀alkyl and aryl-C₀₋₄alkyl, wherein alkyl is optionally substituted with one to four substituents independently chosen from R^{a} and aryl is optionally substituted with one to four substituents independently chosen from R^{b}.

In one embodiment of the invention, R⁶ is chosen from: hydrogen, hydroxyl, C₁₋₄alkyl, and halogen. In a variant of this embodiment, R⁶ is chosen from: hydrogen, hydroxyl, and halogen.

Compounds of Formula I may contain one or more asymmetric centers and can thus occur as racemates and racemic mixtures, single enantiomers, stereoisomers, diastereomeric mixtures and individual diastereomers. The present invention is meant to comprehend all such isomeric forms of the compounds of Formula I.

Some of the compounds described herein contain olefinic double bonds, and unless specified otherwise, are meant to include both E and Z geometric isomers.

Tautomers are defined as compounds that undergo rapid proton shifts from one atom of the compound to another atom of the compound. Some of the compounds described herein may exist as tautomers with different points of attachment of hydrogen. Such an example may be a ketone and its enol form known as keto-enol tautomers. The individual tautomers as well as mixture thereof are encompassed with compounds of Formula I.

Compounds of the Formula I may be separated into diastereoisomeric pairs of enantiomers or stereoisomers by, for example, fractional crystallization from a suitable solvent, for example MeOH or ethyl acetate or a mixture thereof. The pair of enantiomers or stereoisomers thus obtained may be separated into individual stereoisomers by conventional means, for example by the use of an optically active amine as a resolving agent or on a chiral HPLC column.

Alternatively, any enantiomer of a compound of the general Formula I may be obtained by stereospecific synthesis using optically pure starting materials or reagents of known configuration.

Furthermore, some of the crystalline forms for compounds of the present invention may exist as polymorphs and as such are intended to be included in the present invention. In addition, some of the compounds of the instant invention may form solvates with water or common organic solvents. Such solvates are encompassed within the scope of this invention.

It is generally preferable to administer compounds of the present invention as enantiomerically pure formulations. Racemic mixtures can be separated into their individual enantiomers by any of a number of conventional methods. These include chiral chromatography, derivatization with a chiral auxiliary followed by separation by chromatography or crystallization, and fractional crystallization of diastereomeric salts.

The term “pharmaceutically acceptable salts” refers to salts prepared from pharmaceutically acceptable non-toxic bases or acids including inorganic or organic bases and inorganic or organic acids. Salts derived from inorganic bases can be chosen from aluminum, ammonium, calcium, copper, ferric, ferrous, lithium, magnesium, manganic salts, manganous, potassium, sodium, zinc, and the like, such as for example, ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations.

It will be understood that, as used herein, references to the compounds of Formula I are meant to also include the pharmaceutically acceptable salts.

Compounds of the present invention are modulators of the CB1 receptor. In particular, the compounds of structural formula I are antagonists or inverse agonists of the CB1 receptor.

An “agonist” is a compound (hormone, neurotransmitter or synthetic compound) which binds to a receptor and mimics the effects of the endogenous regulatory compound, such as contraction, relaxation, secretion, change in enzyme activity, etc. An “antagonist” is a compound, devoid of intrinsic regulatory activity, which produces effects by interfering with the binding of the endogenous agonist or inhibiting the action of an agonist. An “inverse agonist” is a compound which acts on a receptor but produces the opposite effect produced by the agonist of the particular receptor.

Compounds of this invention are modulators of the CB1 receptor and as such are useful as centrally acting drugs in the treatment of psychosis, memory deficits, cognitive disorders, migraine, neuropathy, neuro-inflammatory disorders including multiple sclerosis and Guillain-Barre syndrome and the inflammatory sequelae of viral encephalitis, cerebral vascular accidents, and head trauma, anxiety disorders, stress, epilepsy, Parkinson's disease, movement disorders, and schizophrenia. The compounds are also useful for the treatment of substance abuse disorders, such as for example, to opiates, alcohol, marijuana, and nicotine. The compounds are also useful for the treatment of obesity or eating disorders associated with excessive food intake and complications associated therewith, including left ventricular hypertrophy. The compounds are also useful for the treatment of constipation and chronic intestinal pseudo-obstruction. The compounds are also useful for the treatment of cirrhosis of the liver. The compounds are also useful for the treatment of asthma.

The terms “administration of” and or “administering a” compound should be understood to mean providing a compound of the invention or a prodrug of a compound of the invention to the individual in need of treatment.

The administration of the compound of structural formula I in order to practice the present methods of therapy is carried out by administering an effective amount of the compound of structural formula I to the mammalian patient in need of such treatment or prophylaxis. The need for a prophylactic administration according to the methods of the present invention is determined by the use of well known risk factors. The effective amount of an individual compound is determined, in the final analysis, by the physician or veterinarian in charge of the case, but depends on factors such as the exact disease to be treated, the severity of the disease and other diseases or conditions from which the patient suffers, the chosen route of administration other drugs and treatments which the patient may concomitantly require, and other factors in the physician's judgment.

The utilities of the present compounds in these diseases or disorders may be demonstrated in animal disease models that have been reported in the literature. The following are examples of such animal disease models: a) suppression of food intake and resultant weight loss in rats (Life Sciences 1998, 63, 113-117); b) reduction of sweet food intake in marmosets (Behavioural Pharm. 1998, 9, 179-181); c) reduction of sucrose and ethanol intake in mice (Psychopharm. 1997, 132, 104-106); d) increased motor activity and place conditioning in rats (Psychopharm. 1998, 135, 324-332; Psychopharmacol 2000, 151: 25-30); e) spontaneous locomotor activity in mice (J. Pharm. Exp. Ther. 1996, 277, 586-594); f) reduction in opiate self-administration in mice (Sci. 1999, 283, 401-404); g) bronchial hyperresponsiveness in sheep and guinea pigs as models for the various phases of asthma (for example, see W. M. Abraham et al., “α₄-Integrins mediate antigen-induced late bronchial responses and prolonged airway hyperresponsiveness in sheep.” J. Clin. Invest. 93, 776 (1993) and A. A. Y. Milne and P. P. Piper, “Role of VLA-4 integrin in leucocyte recruitment and bronchial hyperresponsiveness in the guinea-pig.” Eur. J. Pharmacol., 282, 243 (1995)); h) mediation of the vasodilated state in advanced liver cirrhosis induced by carbon tetrachloride (Nature Medicine, 2001, 7 (7), 827-832); i) amitriptyline-induced constipation in cynomolgus monkeys is beneficial for the evaluation of laxatives (Biol. Pharm. Bulletin (Japan), 2000, 23(5), 657-9); j) neuropathology of paediatric chronic intestinal pseudo-obstruction and animal models related to the neuropathology of paediatric chronic intestinal pseudo-obstruction (Journal of Pathology (England), 2001, 194 (3), 277-88).

The magnitude of prophylactic or therapeutic dose of a compound of Formula I will, of course, vary with the nature of the severity of the condition to be treated and with the particular compound of Formula I and its route of administration. It will also vary according to the age, weight and response of the individual patient. In general, the daily dose range lie within the range of from about 0.001 mg to about 100 mg per kg body weight of a mammal, such as, for example, from 0.01 mg to about 50 mg per kg, and further from 0.1 to 10 mg per kg, in single or divided doses. On the other hand, it may be necessary to use dosages outside these limits in some cases.

For use where a composition for intravenous administration is employed, a suitable dosage range is from about 0.001 mg to about 100 mg (such as for example from 0.01 mg to about 50 mg, further from 0.1 mg to 10 mg) of a compound of Formula I per kg of body weight per day.

In the case where an oral composition is employed, a suitable dosage range is, e.g. from about 0.01 mg to about 1000 mg of a compound of Formula I per day, such as for example from about 0.1 mg to about 10 mg per day. For oral administration, the compositions are can be provided in the form of tablets containing from 0.01 to 1,000 mg, such as for example from 0.01, 0.05, 0.1, 0.5, 1, 2.5, 5, 10, 15, 20, 25, 30, 40, 50, 100, 250, 500, 750 or 1000 milligrams of the active ingredient for the symptomatic adjustment of the dosage to the patient to be treated.

Another aspect of the present invention provides pharmaceutical compositions which comprises a compound of Formula I and a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of Formula I, additional active ingredient(s), and pharmaceutically acceptable excipients.

Any suitable route of administration may be employed for providing a mammal, especially a human, with an effective dosage of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like.

The pharmaceutical compositions of the present invention comprise a compound of Formula I as an active ingredient or a pharmaceutically acceptable salt thereof, and may also contain a pharmaceutically acceptable carrier and optionally other therapeutic ingredients. By “pharmaceutically acceptable” it is meant the carrier, diluent or excipient must be compatible with the other ingredients of the formulation and not deleterious to the recipient thereof. In particular, the term “pharmaceutically acceptable salts” refers to salts prepared from pharmaceutically acceptable non-toxic bases or acids including inorganic bases or acids and organic bases or acids.

The compositions include compositions suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

For administration by inhalation, the compounds of the present invention are conveniently delivered in the form of an aerosol spray presentation from pressurized packs or nebulizers. The compounds may also be delivered as powders which may be formulated and the powder composition may be inhaled with the aid of an insufflation powder inhaler device. Non-limiting examples of delivery systems for inhalation are metered dose inhalation (MDI) aerosol, which may be formulated as a suspension or solution of a compound of Formula I in suitable propellants, such as fluorocarbons or hydrocarbons and dry powder inhalation (DPI) aerosol, which may be formulated as a dry powder of a compound of Formula I with or without additional excipients.

Suitable topical formulations of a compound of formula I include transdermal devices, aerosols, creams, solutions, ointments, gels, lotions, dusting powders, and the like. The topical pharmaceutical compositions containing the compounds of the present invention ordinarily include about 0.005% to 5% by weight of the active compound in admixture with a pharmaceutically acceptable vehicle. Transdermal skin patches useful for administering the compounds of the present invention include those well known to those of ordinary skill in that art. To be administered in the form of a transdernal delivery system, the dosage administration will, of course, be continuous rather than intermittent throughout the dosage regimen.

In practical use, the compounds of Formula I can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

In addition to the common dosage forms set out above, the compounds of Formula I may also be administered by controlled release means and/or delivery devices such as those described in U.S. Pat. Nos. 3,845,770; 3,916,899; 3,536,809; 3,598,123; 3,630,200 and 4,008,719.

Pharmaceutical compositions of the present invention suitable for oral administration may be presented as discrete units such as capsules (including timed release and sustained release formulations), pills, cachets, powders, granules or tablets each containing a predetermined amount of the active ingredient, as a powder or granules or as a solution or a suspension in an aqueous liquid, a non-aqueous liquid, an oil-in-water emulsion or a water-in-oil liquid emulsion, including elixirs, tinctures, solutions, suspensions, syrups and emulsions. Such compositions may be prepared by any of the methods of pharmacy but all methods include the step of bringing into association the active ingredient with the carrier that constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound of the invention moistened with an inert liquid diluent. Desirably, each tablet, cachet, or capsule contains from about 0.01 to about 1,000 mg, such as for example, 0.01, 0.05, 0.1, 0.5, 1, 2.5, 3, 5, 6, 10, 15, 25, 30, 40, 50, 75, 100, 125, 150, 175, 180, 200, 225, 500, 750 and 1,000 milligrams of the compound of the invention, for the symptomatic adjustment of the dosage to the patient to be treated.

Additional suitable means of administration of the compounds of the present invention include injection, intravenous bolus or infusion, intraperitoneal, subcutaneous, intramuscular and topical, with or without occlusion.

Exemplifying the invention is a pharmaceutical composition comprising any of the compounds described above and a pharmaceutically acceptable carrier. Also exemplifying the invention is a pharmaceutical composition made by combining any of the compounds described above and a pharmaceutically acceptable carrier. An illustration of the invention is a process for making a pharmaceutical composition comprising combining any of the compounds described above and a pharmaceutically acceptable carrier.

The dose may be administered in a single daily dose or the total daily dosage may be administered in divided doses of two, three or four times daily. Furthermore, based on the properties of the individual compound selected for administration, the dose may be administered less frequently, e.g., weekly, twice weekly, monthly, etc. The unit dosage will, of course, be correspondingly larger for the less frequent administration.

When administered via intranasal routes, transdermal routes, by rectal or vaginal suppositories, or through a continual intravenous solution, the dosage administration will, of course, be continuous rather than intermittent throughout the dosage regimen.

The following are examples of representative pharmaceutical dosage forms for the compounds of Formula I:

| Injectable Suspension (I.M.) | mg/mL |
|---|---|
| Compound of Formula I | 10 |
| Methylcellulose | 5.0 |
| Tween 80 | 0.5 |
| Benzyl alcohol | 9.0 |
| Benzalkonium chloride | 1.0 |

Water for injection to a total volume of 1 Ml

| Capsule | mg/capsule | |
|---|---|---|
| Compound of | 25 | |
| Formula I | | |
| Lactose Powder | 573.5 | |
| Magnesium Stearate | 1.5 | |
| | 600 | |
| Tablet | mg/tablet | |
| Compound of Formula I | 25 | |
| Microcrystalline | 415 | |
| Cellulose | | |
| Povidone | 14.0 | |
| Pregelatinized Starch | 43.5 | |
| Magnesium Stearate | 2.5 | |
| | 500 | |
| Aerosol | Per canister | |
| Compound of Formula I | 24 | mg |
| Lecithin, NF Liq. Conc. | 1.2 | mg |
| Trichlorofluoromethane, NF | 4.025 | g |
| Dichlorodifluoromethane, NF | 12.15 | g |

Compounds of Formula I may be used in combination with other drugs that are used in the treatment/prevention/suppression or amelioration of the diseases or conditions for which compounds of Formula I are useful. Such other drugs may be administered, by a route and in an amount commonly used therefor, contemporaneously or sequentially with a compound of Formula I. When a compound of Formula I is used contemporaneously with one or more other drugs, a pharmaceutical composition containing such other drugs in addition to the compound of Formula I is preferred. Accordingly, the pharmaceutical compositions of the present invention include those that also contain one or more other active ingredients, in addition to a compound of Formula I. Examples of other active ingredients that may be combined with a compound of Formula I include, but are not limited to: antipsychotic agents, cognition enhancing agents, anti-migraine agents, anti-asthmatic agents, antiinflammatory agents, anxiolytics, anti-Parkinson's agents, anti-epileptics, anorectic agents, serotonin reuptake inhibitors, other anti-obesity agents, as well as antidiabetic agents, lipid lowering agents, and antihypertensive agents which may be administered separately or in the same pharmaceutical compositions.

The present invention also provides a method for the treatment or prevention of a CB1 receptor modulator mediated disease, which method comprises administration to a patient in need of such treatment or at risk of developing a CB1 receptor modulator mediated disease of an amount of a CB1 receptor modulator and an amount of one or more active ingredients, such that together they give effective relief.

In a further aspect of the present invention, there is provided a pharmaceutical composition comprising a CB1 receptor modulator and one or more active ingredients, together with at least one pharmaceutically acceptable carrier or excipient.

Thus, according to a further aspect of the present invention there is provided the use of a CB1 receptor modulator and one or more active ingredients for the manufacture of a medicament for the treatment or prevention of a CB1 receptor modulator mediated disease. In a further or alternative aspect of the present invention, there is therefore provided a product comprising a CB1 receptor modulator and one or more active ingredients as a combined preparation for simultaneous, separate or sequential use in the treatment or prevention of CB1 receptor modulator mediated disease. Such a combined preparation may be, for example, in the form of a twin pack.

It will be appreciated that for the treatment or prevention of eating disorders, including obesity, bulimnia nervosa and compulsive eating disorders, a compound of the present invention may be used in conjunction with other anorectic agents.

The present invention also provides a method for the treatment or prevention of eating disorders, which method comprises administration to a patient in need of such treatment an amount of a compound of the present invention and an amount of an anorectic agent, such that together they give effective relief.

Suitable anorectic agents of use in combination with a compound of the present invention include, but are not limited to, aminorex, amphechloral, amphetamine, benzphetamine, chlorphentermine, clobenzorex, cloforex, clominorex, clortermine, cyclexedrine, dexfenfluramine, dextroamphetamine, diethylpropion, diphemethoxidine, N-ethylamphetamine, fenbutrazate, fenfluramine, fenisorex, fenproporex, fludorex, fluminorex, furfurylmethylamphetamine, levamfetamine, levophacetoperane, mazindol, mefenorex, metamfepramone, methamphetamine, norpseudoephedrine, pentorex, phendimetrazine, phenmetrazine, phentermine, phenylpropanolamine, picilorex and sibutramine; and pharmaceutically acceptable salts thereof.

One non-limiting class of anorectic agents includes the halogenated amphetamine derivatives, such as for example, chlorphentermine, cloforex, clortermine, dexfenfluramine, fenfluramine, picilorex and sibutramine; and pharmaceutically acceptable salts thereof.

One embodiment includes the combination of a compound in accordance with the invention admixed with halogenated amphetamine derivatives selected from fenfluramine, dexfenfluramine, and pharmaceutically acceptable salts thereof.

The present invention also provides a method for the treatment or prevention of obesity, which method comprises administration to a patient in need of such treatment an amount of a compound of the present invention and an amount of another agent useful in treating obesity and obesity-related conditions, such that together they give effective relief.

Suitable anti-obesity agents of use in combination with a compound of the present invention, include, but are not limited to:

(a) anti-diabetic agents such as (1) PPARγ agonists such as glitazones (e.g. ciglitazone; darglitazone; englitazone; isaglitazone (MCC-555); pioglitazone; rosiglitazone; troglitazone; BRLA9653; CLX-0921; 5-BTZD, and GW-0207, LG-100641, and LY-300512, and the like and compounds disclosed in WO97/10813, 97/27857, 97/28115, 97/28137, 97/27847, 03/000685, and 03/027112; (2) biguanides such as buformin; metformin; and phenformin, and the like; (3) protein tyrosine phosphatase-1B (PTP-1B) inhibitors; (4) sulfonylureas such as acetohexamide; chlorpropamide; diabinese; glibenclamide; glipizide; glyburide; glimepiride; gliclazide; glipentide; gliquidone; glisolamide; tolazamide; and tolbutamide, and the like; (5) meglitinides such as repaglinide, and nateglinide, and the like; (6) alpha glucoside hydrolase inhibitors such as acarbose; adiposine; camiglibose; emiglitate; miglitol; voglibose; pradimicin-Q; salbostatin; CKD-711; MDL-25,637; MDL-73,945; and MOR 14, and the like; (7) alpha-amylase inhibitors such as tendamistat, trestatin, and Al-3688, and the like; (8) insulin secreatagogues such as linogliride; and A-4166, and the like; (9) fatty acid oxidation inhibitors, such as clomoxir, and etomoxir, and the like; (10) A2 antagonists, such as midaglizole; isaglidole; deriglidole; idazoxan; earoxan; and fluparoxan, and the like; (11) insulin or insulin mimetics, such as biota, LP-100, novarapid, insulin detemir, insulin lispro, insulin glargine, insulin zinc suspension (lente and ultralente); Lys-Pro insulin, GLP-1 (73-7) (insulintropin); and GLP-1 (7-36)-NH₂), and the like; (12) non-thiazolidinediones such as JT-501, and farglitazar (GW-2570/GI-262579), and the like; (13) PPARα/γdual agonists such as CLX-0940, GW-1536, GW1929, GW-2433, KRP-297, L-796449, LR-90, MK-0767, SB 219994, and reglitazar (JTT-501) and those disclosed in WO 99/16758, WO 99/19313, WO 99/20614, WO 99/38850, WO 00/23415, WO 00/23417, WO 00/23445, WO 00/50414, WO 01/00579, WO 01/79150, WO 02/062799, WO 03/033481, WO 03/033450, WO 03/033453; and (14) other insulin sensitizing drugs; (15) VPAC2 receptor agonists; (16) GLK modulators, such as those disclosed in WO 03/015774; (17) retinoid modulators such as those disclosed in WO 03/000249; (18) GSK 3beta/GSK 3 inhibitors such as 4-[2-(2-bromophenyl)-4-(4-fluorophenyl-1H-imidazol-5-yl]pyridine and those compounds disclosed in WO 03/037869, WO 03/03877, WO 03/037891, WO 03/024447, and the like; (19) glycogen phosphorylase (HGLPa) inhibitors, such as those disclosed in WO 03/037864; (20) ATP consumption promotors such as those disclosed in WO 03/007990; and

(b) lipid lowering agents such as (1) bile acid sequestrants such as, cholestyramine, colesevelem, colestipol, dialkylaminoalkyl derivatives of a cross-linked dextran; Colestid®; LoCholest®; and Questran®, and the like; (2) HMG-CoA reductase inhibitors such as atorvastatin, itavastatin, fluvastatin, lovastatin, pravastatin, rivastatin, rosuvastatin, simvastatin, and ZD4522, and the like; (3) HMG-CoA synthase inhibitors; (4) cholesterol absorption inhibitors such as stanol esters, beta-sitosterol, sterol glycosides such as tiqueside; and azetidinones such as ezetimibe, and the like; (5) acyl coenzyme A -cholesterol acyl transferase (ACAT) inhibitors such as avasimibe, eflucimibe, KY505, SMP 797, and the like; (6) CETP inhibitors such as JTT 705, torcetrapib, CP 532,632, BAY63-2149, SC 591, SC 795, and the like; (7) squalene synthetase inhibitors; (8) anti-oxidants such as probucol, and the like; (9) PPARα agonists such as beclofibrate, benzafibrate, ciprofibrate, clofibrate, etofibrate, fenofibrate, gemcabene, and gemfibrozil, GW 7647, BM 170744, LY518674; and other fibric acid derivatives, such as Atromid®, Lopid® and Tricor®, and the like; (10) FXR receptor modulators such as GW 4064, SR 103912, and the like; (11) LXR receptor modulators such as GW 3965, T9013137, and XTCO179628, and the like; (12) lipoprotein synthesis inhibitors such as niacin; (13) renin angiotensin system inhibitors; (14) PPAR δ partial agonists, such as those disclosed in WO 03/024395; (15) bile acid reabsorption inhibitors, such as BARI 1453, SC435, PHA384640, S8921, AZD7706, and the like; (16) PPARδagonists such as GW 501516, and GW 590735, and the like, such as those disclosed in WO97/28149, WO 01/79197, WO 02/14291, WO 02/46154, WO 02/46176, WO 02/076957, WO 03/016291, WO 03/033493; (17) triglyceride synthesis inhibitors; (18) microsomal triglyceride transport (MTTP) inhibitors, such as inplitapide, LAB687, and CP346086, and the like; (19) transcription modulators; (20) squalene epoxidase inhibitors; (21) low density lipoprotein (LDL) receptor inducers; (22) platelet aggregation inhibitors; (23) 5-LO or FLAP inhibitors; and (24) niacin receptor agonists; (25) PPAR modulators such as those disclosed in WO 01/25181, WO 01/79150, WO 02/79162, WO 02/081428, WO 03/016265, WO 03/033453; (26) niacin-bound chromium, as disclosed in WO 03/039535; (27) substituted acid derivatives disclosed in WO 03/040114; and

(c) anti-hypertensive agents such as (1) diuretics, such as thiazides, including chlorthalidone, chlorthiazide, dichlorophenamide, hydroflumethiazide, indapamide, and hydrochlorothiazide; loop diuretics, such as bumetanide, ethacrynic acid, furosemide, and torsemide; potassium sparing agents, such as amiloride, and triamterene; and aldosterone antagonists, such as spironolactone, epirenone, and the like; (2) beta-adrenergic blockers such as acebutolol, atenolol, betaxolol, bevantolol, bisoprolol, bopindolol, carteolol, carvedilol, celiprolol, esmolol, indenolol, metaprolol, nadolol, nebivolol, penbutolol, pindolol, propanolol, sotalol, tertatolol, tilisolol, and timolol, and the like; (3) calcium channel blockers such as amlodipine, aranidipine, azelnidipine, barnidipine, benidipine, bepridil, cinaldipine, clevidipine, diltiazem, efonidipine, felodipine, gallopamil, isradipine, lacidipine, lemildipine, lercanidipine, nicardipine, nifedipine, nilvadipine, nimodepine, nisoldipine, nitrendipine, manidipine, pranidipine, and verapamil, and the like; (4) angiotensin converting enzyme (ACE) inhibitors such as benazepril; captopril; cilazapril; delapril; enalapril; fosinopril; imidapril; losinopril; moexipril; quinapril; quinaprilat; ramipril; perindopril; perindropril; quanipril; spirapril; tenocapril; trandolapril, and zofenopril, and the like; (5) neutral endopeptidase inhibitors such as omapatrilat, cadoxatril and ecadotril, fosidotril, sampatrilat, AVE7688, ER4030, and the like; (6) endothelin antagonists such as tezosentan, A308165, and YM62899, and the like; (7) vasodilators such as hydralazine, clonidine, minoxidil, and nicotinyl alcohol, and the like; (8) angiotensin II receptor antagonists such as candesartan, eprosartan, irbesartan, losartan, pratosartan, tasosartan, telmisartan, valsartan, and EXP-3137, FI6828K, and RNH6270, and the like; (9) α/β adrenergic blockers as nipradilol, arotinolol and amosulalol, and the like; (10) alpha 1 blockers, such as terazosin, urapidil, prazosin, bunazosin, trimazosin, doxazosin, naftopidil, indoramin, WHIP 164, and XEN010, and the like; (11) alpha 2 agonists such as lofexidine, tiamenidine, moxonidine, rilmenidine and guanobenz, and the like; and (12) aldosterone inhibitors, and the like; and

(d) anti-obesity agents, such as (1) SHT (serotonin) transporter inhibitors, such as paroxetine, fluoxetine, fenfluramine, fluvoxamine, sertraline, and imipramine, and those disclosed in WO 03/00663; (2) NE (norepinephrine) transporter inhibitors, such as GW 320659, despiramine, talsupram, and nomifensine; (3) CB1 (cannabinoind-1 receptor) antagonist/inverse agonists, such as rimonabant (Sanofi Synthelabo), SR-147778 (Sanofi Synthelabo), BAY 65-2520 (Bayer), and SLV 319 (Solvay), and those disclosed in U.S. Pat. Nos. 4,973,587, 5,013,837, 5,081,122, 5,112,820, 5,292,736, 5,532,237, 5,624,941, 6,028,084, and 6,509367; and WO 96/33159, WO97/29079, WO98/31227, WO 98/33765, WO98/37061, WO98/41519, WO98/43635, WO98/43636, WO99/02499, WO00/10967, WO00/10968, WO 01/09120, WO 01/58869, WO 01/64632, WO 01/64633, WO 01/64634, WO 01/70700, WO 01/96330, WO 02/076949, WO 03/006007, WO 03/007887, WO 03/020217, WO 03/026647, WO 03/026648, WO 03/027069, WO 03/027076, WO 03/027114, WO 03/037332, WO 03/040107 and EPO Application No. EP-658546; (4) ghrelin antagonists, such as those disclosed in WO 01/87335, and WO 02/08250; (5) H3 (histamine H3) antagonist/inverse agonists, such as thioperamide, 3-(1H-imidazol-4-yl)propyl N-(4-pentenyl)carbamate), clobenpropit, iodophenpropit, imoproxifan, GT2394 (Gliatech), and A331440, and those disclosed in WO 02/15905; and O-[3-(1H-imidazol-4-yl)propanol]carbamates (Kiec-Kononowicz, K. et al., Pharmazie, 55:349-55 (2000)), piperidine-containing histamine H3-receptor antagonists (Lazewska, D. et al., Pharmazie, 56:927-32 (2001), benzophenone derivatives and related compounds (Sasse, A. et al., Arch. Pharm. (Weinheim) 334:45-52 (2001)), substituted N-phenylcarbamates (Reidemeister, S. et al., Pharmazie, 55:83-6 (2000)), and proxifan derivatives (Sasse, A. et al., J. Med. Chem. 43:3335-43 (2000)) and histamine H3 receptor modulators such as those disclosed in WO 03/024928 and WO 03/024929; (6) melanin-concentrating hormone 1 receptor (MCH1R) antagonists, such as T-226296 (Takeda), SNP-7941 (Synaptic), and those disclosed WO 01/21169, WO 01/82925, WO 01/87834, WO 02/051809, WO 02/06245, WO 02/076929, WO 02/076947, WO 02/04433, WO 02/51809, WO 02083134, WO 02/094799, WO 03/004027, WO 03/13574, WO 03/15769, WO 03/028641, WO 03/035624, WO 03/033476, WO 03/033480; and Japanese Patent Application Nos. JP 13226269, and JP 1437059; (7) MCH2R (melanin concentrating hormone 2R) agonist/antagonists; (8) NPY1 (neuropeptide Y Y1) antagonists, such as BIBP3226, J-115814, BIBO 3304, LY-357897, CP-671906, and GI-264879A; and those disclosed in U.S. Pat. No. 6,001,836; and WO 96/14307, WO 01/23387, WO 99/51600, WO 01/85690, WO 01/85098, WO 01/85173, and WO 01/89528; (9) NPY5 (neuropeptide Y Y5) antagonists, such as 152,804, GW-569180A, GW-594884A, GW-587081X, GW-548118X; FR 235,208; FR226928, FR 240662, FR252384; 1229U91, GI-264879A, CGP71683A, LY-377897, LY366377, PD-160170, SR-120562A, SR-120819A, JCF-104, and H409/22; and those compounds disclosed in U.S. Pat. Nos. 6,140,354, 6,191,160, 6,258,837, 6,313,298, 6,326,375, 6,329,395, 6,335,345, 6,337,332, 6,329,395, and 6,340,683; European Patent Nos. EP-01010691, and EP-01044970; and PCT Publication Nos. WO 97/19682, WO 97/20820, WO 97/20821, WO 97/20822, WO 97/20823, WO 98/27063, WO 00/107409, WO 00/185714, WO 00/185730, WO 00/64880, WO 00/68197, WO 00/69849, WO 01/09120, WO 01/14376, WO 01/85714, WO 01/85730, WO 01/07409, WO 01/02379, WO 01/02379, WO 01/23388, WO 01/23389, WO 01/44201, WO 01/62737, WO 01/62738, WO 01/09120, WO 02/20488, WO 02/22592, WO 02/48152, WO 02/49648, WO 02/051806, WO 02/094789, WO 03/009845, WO 03/014083, WO 03/022849, WO 03/028726; and Norman et al., J. Med. Chem. 43:4288-4312 (2000); (10) leptin, such as recombinant human leptin (PEG-OB, Hoffman La Roche) and recombinant methionyl human leptin (Amgen); (11) leptin derivatives, such as those disclosed in U.S. Pat. Nos. 5,552,524; 5,552,523; 5,552,522; 5,521,283; and WO 96/23513; WO 96/23514; WO 96/23515; WO 96/23516; WO 96/23517; WO 96/23518; WO 96/23519; and WO 96/23520; (12) opioid antagonists, such as nalmefene (Revex®), 3-methoxynaltrexone, naloxone, and naltrexone; and those disclosed in WO 00/21509; (13) orexin antagonists, such as SB-334867-A; and those disclosed in WO 01/96302, WO 01/68609, WO 02/44172, WO 02/51232, WO 02/51838, WO 02/089800, WO 02/090355, WO 03/023561, WO 03/032991, WO 03/037847; (14) BRS3 (bombesin receptor subtype 3) agonists; (15) CCK-A (cholecystokinin-A) agonists, such as AR-R 15849, GI 181771, JMV-180, A-71378, A-71623 and SR146131, and those disclosed in U.S. Pat. No. 5,739,106; (16) CNTF (ciliary neurotrophic factors), such as GI-181771 (Glaxo-SmithKline); SR146131 (Sanofi Synthelabo); butabindide; and PD170,292, PD 149164 (Pfizer); (17) CNTF derivatives, such as axokine (Regeneron); and those disclosed in WO 94/09134, WO 98/22128, and WO 99/43813; (18) GHS (growth hormone secretagogue receptor) agonists, such as NN703, hexarelin, MK-0677, SM-130686, CP424,391, L-692,429 and L-163,255, and those disclosed in U.S. Pat. No. 6,358,951, U.S. Patent Application Nos. 2002/049196 and 2002/022637; and WO 01/56592, and WO 02/32888; (19) 5HT2c (serotonin receptor 2c) agonists, such as BVT933, DPCA37215, IK264; PNU 22394; WAY161503, R-1065, and YM 348; and those disclosed in U.S. Pat. No. 3,914,250; and WO 02/36596, WO 02/48124, WO 02/10169, WO 01/66548, WO 02/44152; WO 02/51844, WO 02/40456, and WO 02/40457; (20) Mc3r (melanocortin 3 receptor) agonists; (21) Mc4r (melanocortin 4 receptor) agonists, such as CHIR86036 (Chiron); ME-10142, ME-10145, and HS-131 (Melacure), and those disclosed in WO 99/64002, WO 00/74679, WO 01/991752, WO 01/0125192, WO 01/52880, WO 01/74844, WO 01/70708, WO 01/70337, WO 01/91752, WO 02/059095, WO 02/059107, WO 02/059108, WO 02/059117, WO 02/06276, WO 02/12166, WO 02/11715, WO 02/12178, WO 02/15909, WO 02/38544, WO 02/068387, WO 02/068388, WO 02/067869, WO 02/081430, WO 03/06604, WO 03/007949, WO 03/009847, WO 03/009850, WO 03/013509, and WO 03/031410; (22) monoamine reuptake inhibitors, such as sibutratmine (Meridia ®/Reductil®) and salts thereof, and those compounds disclosed in U.S. Pat. Nos. 4,746,680, 4,806,570, and 5,436,272, and U.S. Patent Publication No. 2002/0006964, and WO 01/27068, and WO 01/62341; (23) serotonin reuptake inhibitors, such as dexfenfluramine, fluoxetine, and those in U.S. Pat. No. 6,365,633, and WO 01/27060, and WO 01/162341; (24) GLP-1 (glucagon-like peptide 1) agonists; (25) Topiramate (Topimax®); (26) phytopharm compound 57 (CP 644,673); (27) ACC2 (acetyl-CoA carboxylase-2) inhibitors; (28)β3 (beta adrenergic receptor 3) agonists, such as AD9677/TAK677 (Dainippon/Takeda), CL-316,243, SB 418790, BRL-37344, L-796568, BMS-196085, BRL-35135A, CGP12177A, BTA-243, GW 427353, Trecadrine, Zeneca D7114, N-5984 (Nisshin Kyorin), LY-377604 (Lilly), and SR 59119A, and those disclosed in US Pat. No. 5,705,515, U.S. Pat. No. 5,451,677; and WO94/18161, WO95/29159, WO97/46556, WO98/04526 and WO98/32753, WO 01/74782, WO 02/32897, WO 03/014113, WO 03/016276, WO 03/016307, WO 03/024948, WO 03/024953; and WO 03/037881; (29) DGAT1 (diacylglycerol acyltransferase 1) inhibitors; (30) DGAT2 (diacylglycerol acyltransferase 2)inhibitors; (31) FAS (fatty acid synthase) inhibitors, such as Cerulenin and C75; (32) PDE (phosphodiesterase) inhibitors, such as theophylline, pentoxifylline, zaprinast, sildenafil, amrinone, milrinone, cilostamide, rolipram, and cilomilast, as well as those described in WO 03/037432, WO 03/037899; (33) thyroid hormone β agonists, such as KB-2611 (KaroBioBMS), and those disclosed in WO 02/15845; and Japanese Patent Application No. JP 2000256190; (34) UCP-1 (uncoupling protein 1), 2, or 3 activators, such as phytanic acid, 4-[(E)-2-(5,6,7,8-tetrahydro-5,5,8,8-tetramethyl-2-napthalenyl)-1-propenyl]benzoic acid (TTNPB), and retinoic acid; and those disclosed in WO 99/00123; (35) acyl-estrogens, such as oleoyl-estrone, disclosed in del Mar-Grasa, M. et al., Obesity Research, 9:202-9 (2001); (36) glucocorticoid antagonists; (37) 11β HSD-1 (11-beta hydroxy steroid dehydrogenase type 1) inhibitors, such as BVT 3498, BVT 2733, 3-(1-adamantyl)-4-ethyl-5-ethylthio)4H-1,2,4-triazole, 3-(1-adamantyl)-5-(3,4,5-trimethoxyphenyl)-4-methyl-4H-1,2,4-triazole, 3-adamantanyl-4,5,6,7,8,9,10,11,12,3a-decahydro-1,2,4-triazolo[4,3-a][11]annulene, and those compounds disclosed in WO 01/90091, WO 01/90090, WO 01/90092 and WO 02/072084; (38) SCD-1 (stearoyl-CoA desaturase-1) inhibitors; (39) dipeptidyl peptidase IV (DP-IV) inhibitors, such as isoleucine thiazolidide, valine pyrrolidide, NVP-DPP728, LAF237, P93/01, TSL 225, TMC-2A/2B/2C, FE 999011, P9310/K364, VIP 0177, SDZ 274-444; and the compounds disclosed in WO 02/083128, WO 02/062764, WO 03/000180, WO 03/000181, WO 03/000250, WO 03/002530, WO 03/002531, WO 03/002553, WO 03/002593, WO 03/004498, WO 03/004496,WO 03/017936, WO 03/024942, WO 03/024965, WO 03/033524, WO 03/037327and EP 1 258 476; (40) lipase inhibitors, such as tetrahydrolipstatin (orlistat/Xenical®), Triton WR1339, RHC80267, lipstatin, teasaponin, and diethylumbelliferyl phosphate, FL-386, WAY-121898, Bay-N-3176, valilactone, esteracin, ebelactone A, ebelactone B, and RHC 80267, and those disclosed in WO 01/77094, and U.S. Pat. Nos. 4,598,089, 4,452,813, 5,512,565, 5,391,571, 5,602,151, 4,405,644, 4,189,438, and 4,242,453; (41) fatty acid transporter inhibitors; (42) dicarboxylate transporter inhibitors; (43) glucose transporter inhibitors; and (44) phosphate transporter inhibitors; (45) anorectic bicyclic compounds such as 1426 (Aventis) and 1954 (Aventis), and the compounds disclosed in WO 00/18749, WO 01/32638, WO 01/62746, WO 01/62747, and WO 03/015769; (46) peptide YY and PYY agonists such as those disclosed in WO 03/026591; (47) lipid metabolism modulators such as maslinic acid, erythrodiol, ursolic acid uvaol, betulinic acid, betulin, and the like and compounds disclosed in WO 03/011267; (48) transcription factor modulators such as those disclosed in WO 03/026576; (49) Mc5r (melanocortin 5 receptor) modulators, such as those disclosed in WO 97/19952, WO 00/15826, WO 00/15790, US 20030092041, and the like.

Specific NPY5 antagonists of use in combination with a compound of the present invention are selected from the group consisting of: 3-oxo-N-(5-phenyl-2-pyrazinyl)-spiro[isobenzofuran-1(3H),4′-piperidine]-1′-carboxamide, 3-oxo-N-7-trifluoromethylpyrido[3,2-b]pyridin-2-yl)spiro-[isobenzofuran-1(3H),4′-piperidine]-1′-carboxamide, N-[5-3-fluorophenyl)-2-pyrimidinyl]-3-oxospiro-[isobenzofuran-1(3H),4-piperidine]-1′-carboxamide, trans-3′-oxo-N-(5-phenyl-2-pyrimidinyl)spiro[cyclohexane-1,1′(3′H)-isobenzofuran]4-carboxamide, trans-3′-oxo-N-[1-(3-quinolyl)4-imidazolyl]spiro[cyclohexane-1,1′(3′H)-isobenzofuran]-4-carboxamide, trans-3-oxo-N-(5-phenyl-2-pyrazinyl)spiro[4-azaiso-benzofuran-1(3H),1′-cyclohexane]-4′-carboxamide, trans-N-[5-(3-fluorophenyl)-2-pyrimidinyl]-3-oxospiro[5-azaisobenzofuran-1(3H),1′-cyclohexane]4′-carboxamide, trans-N-[5-(2-fluorophenyl)-2-pyrimidinyl]-3-oxospiro[5-azaisobenzofuran-1(3H),1′-cyclohexane]4′-carboxamide, trans-N-[1-(3,5-difluorophenyl)-4-imidazolyl]-3-oxospiro[7-azaisobenzofuran-1(3H),1′-cyclohexane]4′-carboxamide, trans-3-oxo-N-(1-phenyl-4-pyrazolyl)spiro[4-azaisobenzofuran-1(3H),1′-cyclohexane]4′-carboxamide, trans-N-[1-(2-fluorophenyl)-3-pyrazolyl]-3-oxospiro[6-azaisobenzofuran-1(3H),1′-cyclohexane]-4′-carboxamide, trans-3-oxo-N-(1-phenyl-3-pyrazolyl)spiro[6-azaisobenzofuran-1(3H),1′-cyclohexane]-4′-carboxamide, trans-3-oxo-N-2-phenyl-1,2,3-triazol-4-yl)spiro[6-azaisobenzofuran-1(3H),1′-cyclohexane]-4′-carboxamide, and pharmaceutically acceptable salts and esters thereof.

“Obesity” is a condition in which there is an excess of body fat. The operational definition of obesity is based on the Body Mass Index (BMI), which is calculated as body weight per height in meters squared (kg/m²). “Obesity” refers to a condition whereby an otherwise healthy subject has a Body Mass Index (BMI) greater than or equal to 30 kg/m², or a condition whereby a subject with at least one co-morbidity has a BMI greater than or equal to 27 kg/m². An “obese subject” is an otherwise healthy subject with a Body Mass Index (BWM) greater than or equal to 30 kg/m² or a subject with at least one co-morbidity with a BMI greater than or equal to 27 kg/m². A “subject at risk for obesity” is an otherwise healthy subject with a BMI of 25 kg/m² to less than 30 kg/m² or a subject with at least one co-morbidity with a BMI of 25 kg/m² to less than 27 kg/m².

The increased risks associated with obesity occur at a lower Body Mass Index (BMI) in Asians. In Asian countries, including Japan, “obesity” refers to a condition whereby a subject with at least one obesity-induced or obesity-related co-morbidity that requires weight reduction or that would be improved by weight reduction, has a BMI greater than or equal to 25 kg/m². In Asian countries, including Japan, an “obese subject” refers to a subject with at least one obesity-induced or obesity-related co-morbidity that requires weight reduction or that would be improved by weight reduction, with a BMI greater than or equal to 25 kg/m². In Asian countries, a “subject at risk of obesity” is a subject with a BMI of greater than 23 kg/m² to less than 25 kg/m².

As used herein, the term “obesity” is meant to encompass all of the above definitions of obesity.

Obesity-induced or obesity-related co-morbidities include, but are not limited to, diabetes, non-insulin dependent diabetes mellitus—type 2, impaired glucose tolerance, impaired fasting glucose, insulin resistance syndrome, dyslipidemia, hypertension, hyperuricacidemia, gout, coronary artery disease, myocardial infarction, angina pectoris, sleep apnea syndrome, Pickwickian syndrome, fatty liver, cerebral infarction, cerebral thrombosis, transient ischemic attack, orthopedic disorders, arthritis deformans, lumbodynia, emmeniopathy, and infertility. A subset of the co-morbidities include: hypertension, hyperlipidemia, dyslipidemia, glucose intolerance, cardiovascular disease, sleep apnea, diabetes mellitus, and other obesity-related conditions.

“Treatment” (of obesity and obesity-related disorders) refers to the administration of the compounds of the present invention to reduce or maintain the body weight of an obese subject. One outcome of treatment may be reducing the body weight of an obese subject relative to that subject's body weight immediately before the administration of the compounds of the present invention. Another outcome of treatment may be preventing body weight regain of body weight previously lost as a result of diet, exercise, or pharmacotherapy. Another outcome of treatment may be decreasing the occurrence of and/or the severity of obesity-related diseases. The treatment may suitably result in a reduction in food or calorie intake by the subject, including a reduction in total food intake, or a reduction of intake of specific components of the diet such as carbohydrates or fats; and/or the inhibition of nutrient absorption; and/or the inhibition of the reduction of metabolic rate; and in weight reduction in patients in need thereof. The treatment may also result in an alteration of metabolic rate, such as an increase in metabolic rate, rather than or in addition to an inhibition of the reduction of metabolic rate; and/or in minimization of the metabolic resistance that normally results from weight loss.

“Prevention” (of obesity and obesity-related disorders) refers to the administration of the compounds of the present invention to reduce or maintain the body weight of a subject at risk of obesity. One outcome of prevention may be reducing the body weight of a subject at risk of obesity relative to that subject's body weight immediately before the administration of the compounds of the present invention. Another outcome of prevention may be preventing body weight regain of body weight previously lost as a result of diet, exercise, or pharmacotherapy. Another outcome of prevention may be preventing obesity from occurring if the treatment is administered prior to the onset of obesity in a subject at risk of obesity. Another outcome of prevention may be decreasing the occurrence and/or severity of obesity-related disorders if the treatment is administered prior to the onset of obesity in a subject at risk of obesity. Moreover, if treatment is commenced in already obese subjects, such treatment may prevent the occurrence, progression or severity of obesity-related disorders, such as, but not limited to, arteriosclerosis, Type II diabetes, polycystic ovarian disease, cardiovascular diseases, osteoarthritis, dermatological disorders, hypertension, insulin resistance, hypercholesterolemia, hypertriglyceridemia, and cholelithiasis.

The obesity-related disorders herein are associated with, caused by, or result from obesity. Examples of obesity-related disorders include overeating and bulimia, hypertension, diabetes, elevated plasma insulin concentrations and insulin resistance, dyslipidemias, hyperlipidemia, endometrial, breast, prostate and colon cancer, osteoarthritis, obstructive sleep apnea, cholelithiasis, gallstones, heart disease, abnormal heart rhythms and arrythmias, myocardial infarction, congestive heart failure, coronary heart disease, sudden death, stroke, polycystic ovarian disease, craniopharyngioma, the Prader-Willi Syndrome, Frohlich's syndrome, GH-deficient subjects, normal variant short stature, Tumer's syndrome, and other pathological conditions showing reduced metabolic activity or a decrease in resting energy expenditure as a percentage of total fat-free mass, e.g, children with acute lymphoblastic leukemia. Further examples of obesity-related disorders are metabolic syndrome, also known as syndrome X, insulin resistance syndrome, sexual and reproductive dysfunction, such as infertility, hypogonadism in males and hirsutism in females, gastrointestinal motility disorders, such as obesity-related gastro-esophageal reflux, respiratory disorders, such as obesity-hypoventilation syndrome (Pickwickian syndrome), cardiovascular disorders, inflammation, such as systemic inflammation of the vasculature, arteriosclerosis, hypercholesterolemia, hyperuricaemia, lower back pain, gallbladder disease, gout, and kidney cancer. The compounds of the present invention are also useful for reducing the risk of secondary outcomes of obesity, such as reducing the risk of left ventricular hypertrophy.

The compounds of formula I and II are also useful for treating or preenting obesity and obesity-related disorders in cats and dogs. As such, the term “mammal” includes companion animals such as cats and dogs.

The term “diabetes,” as used herein, includes both insulin-dependent diabetes mellitus (i.e., IDDM, also known as type I diabetes) and non-insulin-dependent diabetes mellitus (i.e., NHDDM, also known as Type II diabetes. Type I diabetes, or insulin-ependent diabetes, is the result of an absolute deficiency of insulin, the hormone which regulates glucose utilization. Type II diabetes, or insulin-independent diabetes (i.e., non-insulin-dependent diabetes mellitus), often occurs in the face of normal, or even elevated levels of insulin and appears to be the result of the inability of tissues to respond appropriately to insulin. Most of the Type II diabetics are also obese. The compounds of the present invention are useful for treating both Type I and Type II diabetes. The compounds are especially effective for treating Type II diabetes. The compounds of the present invention are also useful for treating and/or preventing gestational diabetes mellitus.

It will be appreciated that for the treatment or prevention of migraine, a compound of the present invention may be used in conjunction with other anti-migraine agents, such as ergotamines or 5-HT₁ agonists, especially sumatriptan, naratriptan, zolmatriptan or rizatriptan.

It will be appreciated that for the treatment of depression or anxiety, a compound of the present invention may be used in conjunction with other anti-depressant or anti-anxiety agents.

Suitable classes of anti-depressant agents include norepinephrine reuptake inhibitors, selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, a-adrenoreceptor antagonists, neurokinin-1 receptor antagonists and atypical anti-depressants.

Suitable norepinephrine reuptake inhibitors include tertiary amine tricyclics and secondary amine tricyclics. Suitable examples of tertiary amine tricyclics include: amitriptyline, clomipramine, doxepin, imiprarnine and trimipramine, and pharmaceutically acceptable salts thereof. Suitable examples of secondary amine tricyclics include: amoxapine, desipramine, maprotiline, nortriptyline and protriptyline, and pharmaceutically acceptable salts thereof.

Suitable selective serotonin reuptake inhibitors include: fluoxetine, fluvoxamine, paroxetine, imipramine and sertraline, and pharmaceutically acceptable salts thereof.

Suitable monoamine oxidase inhibitors include: isocarboxazid, phenelzine, tranylcypromine and selegiline, and pharmaceutically acceptable salts thereof.

Suitable reversible inhibitors of monoamine oxidase include: moclobemide, and pharmaceutically acceptable salts thereof.

Suitable serotonin and noradrenaline reuptake inhibitors of use in the present invention include: venlafaxine, and pharmaceutically acceptable salts thereof.

Suitable CRF antagonists include those compounds described in International Patent Specification Nos. WO 94/13643, WO 94/13644, WO 94/13661, WO 94/13676 and WO 94/13677. Still further, neurokinin-1 (NK-1) receptor antagonists may be favorably employed with the CB1 receptor modulators of the present invention. NK-1 receptor antagonists of use in the present invention are fully described, for example, in U.S. Pat. Nos. 5,162,339, 5,232,929, 5,242,930, 5,373,003, 5,387,595, 5,459,270, 5,494,926, 5,496,833, 5,637,699; European Patent Publication Nos. EP 0 360 390, 0 394 989, 0 428 434, 0 429 366, 0 430 771, 0 436 334, 0 443 132, 0 482 539, 0 498 069, 0 499 313, 0 512 901, 0 512 902, 0 514 273, 0514 274, 0 514 275, 0 514 276, 0 515 681, 0 517 589, 0 520 555, 0 522 808, 0 528 495, 0 532 456, 0 533 280, 0 536 817, 0 545 478, 0 558 156, 0 577 394, 0 585 913, 0 590 152, 0 599 538, 0 610 793, 0 634 402, 0 686 629, 0 693 489, 0 694 535, 0 699 655, 0 699 674, 0 707 006, 0 708 101, 0 709 375, 0 709 376, 0 714 891, 0 723 959, 0 733 632 and 0 776 893; PCT International Patent Publication Nos. WO 90/05525, 90/05729, 91/09844, 91/18899, 92/01688, 92/06079, 92/12151, 92/15585, 92/17449, 92/20661, 92/20676, 92/21677, 92/22569, 93/00330, 93/00331, 93/01159, 93/01165, 93/01169, 93/01170, 93/06099, 93/09116, 93/10073, 93/14084, 93/14113, 93/18023, 93/19064, 93/21155, 93/21181, 93/23380, 93/24465, 94/00440, 94/01402, 94/02461, 94/02595, 94/03429, 94/03445, 94/04494, 94/04496, 94/05625, 94/07843, 94/08997, 94/10165, 94/10167, 94/10168, 94/10170, 94/11368, 94/13639, 94/13663, 94/14767, 94/15903, 94/19320, 94/19323, 94/20500, 94/26735, 94/26740, 94/29309, 95/02595, 95/04040, 95/04042, 95/06645, 95/07886, 95/07908, 95/08549, 95/11880, 95/14017, 95/15311, 95/16679, 95/17382, 95/18124, 95/18129, 95/19344, 95/20575, 95/21819, 95/22525, 95/23798, 95/26338, 95/28418, 95/30674, 95/30687, 95/33744, 96/05181, 96/05193, 96/05203, 96/06094, 96/07649, 96/10562, 96/16939, 96/18643, 96/20197, 96/21661, 96/29304, 96/29317, 96/29326, 96/29328, 96/31214, 96/32385, 96/37489, 97/01553, 97/01554, 97/03066, 97/08144, 97/14671, 97/17362, 97/18206, 97/19084, 97/19942, 97/21702, 97/49710, 98/24438-98/24441, 98/24442-98/24445, 02/16343, and 02/16344; and in British Patent Publication Nos. 2 266 529, 2 268 931, 2 269 170, 2 269 590, 2 271 774, 2 292 144, 2 293 168, 2 293 169, and 2 302 689.

Specific neurokinin-1 receptor antagonists of use in the present invention include: (±)-(2R3R,2S3S)-N-{[2-cyclopropoxy-5-(trifluoromethoxy)-phenyl]methyl}-2-phenylpiperidin-3-amine; 2-(R)-(1-(R)-(3,5-bis(trifluoromethyl)phenyl)ethoxy)-43-(5-oxo-1H,4H-1,2,4-triazolo)methyl)-3-(S)-phenyl-morpholine; operpitant; CJ17493; GW597599; GW679769; R673; R067319; R1124; R1204; SSR246977; SSR2400600; T2328; and T2763; or a pharmaceutically acceptable salt thereof.

Suitable atypical anti-depressants include: bupropion, lithium, nefazodone, trazodone and viloxazine, and pharmaceutically acceptable salts thereof.

Suitable classes of anti-anxiety agents include benzodiazepines and 5-HT_{1A} agonists or antagonists, especially 5-HT_{1A} partial agonists, and corticotropin releasing factor (CRF) antagonists.

Suitable benzodiazepines include: alprazolam, chlordiazepoxide, clonazepam, chlorazepate, diazepam, halazepam, lorazepam, oxazepam and prazepam, and pharmaceutically acceptable salts thereof.

Suitable 5-HT_{1A} receptor agonists or antagonists include, for example, the 5-HT_{1A} receptor partial agonists buspirone, flesinoxan, gepirone and ipsapirone, and pharmaceutically acceptable salts thereof.

Suitable corticotropin releasing factor (CRF) antagonists include those previously discussed herein.

As used herein, the term “substance abuse disorders” includes substance dependence or abuse with or without physiological dependence. The substances associated with these disorders are: alcohol, amphetamines (or amphetamine-like substances), caffeine, cannabis, cocaine, hallucinogens, inhalants, marijuana, nicotine, opioids, phencyclidine (or phencyclidine-like compounds), sedative-hypnotics or benzodiazepines, and other (or unknown) substances and combinations of all of the above.

The term “substance abuse disorders” includes drug withdrawal disorders such as alcohol withdrawal with or without perceptual disturbances; alcohol withdrawal delirium; amphetamine withdrawal; cocaine withdrawal; nicotine withdrawal; opioid withdrawal; sedative, hypnotic or anxiolytic withdrawal with or without perceptual disturbances; sedative, hypnotic or anxiolytic withdrawal delirium; and withdrawal symptoms due to other substances. It will be appreciated that reference to treatment of nicotine withdrawal includes the treatment of symptoms associated with smoking cessation.

Other “substance abuse disorders” include substance-induced anxiety disorder with onset during withdrawal; substance-induced mood disorder with onset during withdrawal; and substance-induced sleep disorder with onset during withdrawal.

In particular, compounds of structural formula I are useful for aiding in stopping consumption of tobacco and are useful in treating nicotine dependencies and nicotine withdrawal. The compounds of formula I produce in consumers of nicotine, such as tobacco smokers, a total or partial abstinence from smoking. Further, withdrawal symptoms are lessened and the weight gain that generally accompanies quitting tobacco comsumption is reduced or nonexistent. For smoking cessation, the compound of form I may be used in combination with a nicotine agonist or a partial nicotine agonist, or a monoamine oxidase inhibitor (MAOI), or another active ingredient demonstrating efficacy in aiding cessation of tobacco consumption; for example, an antidepressant such as bupropion, doxepine, ornortriptyline; or an anxiolytic such as buspirone or clonidine.

It will be appreciated that a combination of a conventional antipsychotic drug with a CB1 receptor modulator may provide an enhanced effect in the treatment of mania. Such a combination would be expected to provide for a rapid onset of action to treat a manic episode thereby enabling prescription on an “as needed basis”. Furthermore, such a combination may enable a lower dose of the antispychotic agent to be used without compromising the efficacy of the antipsychotic agent, thereby minimizing the risk of adverse side-effects. A yet further advantage of such a combination is that, due to the action of the CB1 receptor modulator, adverse side-effects caused by the antipsychotic agent such as acute dystonias, dyskinesias, akathesia and tremor may be reduced or prevented.

Thus, according to a further aspect of the present invention there is provided the use of a CB1 receptor modulator and an antipsychotic agent for the manufacture of a medicament for the treatment or prevention of mania.

The present invention also provides a method for the treatment or prevention of mania, which method comprises administration to a patient in need of such treatment or at risk of developing mania of an amount of a CB1 receptor modulator and an amount of an antipsychotic agent, such that together they give effective relief.

In a further aspect of the present invention, there is provided a pharmaceutical composition comprising a CB1 receptor modulator and an antipsychotic agent, together with at least one pharmaceutically acceptable carrier or excipient.

It will be appreciated that the CB1 receptor modulator and the antipsychotic agent may be present as a combined preparation for simultaneous, separate or sequential use for the treatment or prevention of mania. Such combined preparations may be, for example, in the form of a twin pack.

It will be appreciated that when using a combination of the present invention, the CB1 receptor modulator and the antipsychotic agent may be in the same pharmaceutically acceptable carrier and therefore administered simultaneously. They may be in separate pharmaceutical carriers such as conventional oral dosage forms which are taken simultaneously. The term “combination” also refers to the case where the compounds are provided in separate dosage forms and are administered sequentially. Therefore, by way of example, the antipsychotic agent may be administered as a tablet and then, within a reasonable period of time, the CB1 receptor modulator may be administered either as an oral dosage form such as a tablet or a fast-dissolving oral dosage form. By a “fast-dissolving oral formulation” is meant an oral delivery form which when placed on the tongue of a patient, dissolves within about 10 seconds.

Included within the scope of the present invention is the use of CB1 receptor modulators in combination with an antipsychotic agent in the treatment or prevention of hypomania.

It will be appreciated that a combination of a conventional antipsychotic drug with a CB1 receptor modulator may provide an enhanced effect in the treatment of schizophrenic disorders. Such a combination would be expected to provide for a rapid onset of action to treat schizophrenic symptoms thereby enabling prescription on an “as needed basis”. Furthermore, such a combination may enable a lower dose of the CNS agent to be used without compromising the efficacy of the antipsychotic agent, thereby minimizing the risk of adverse side-effects. A yet further advantage of such a combination is that, due to the action of the CB1 receptor modulator, adverse side-effects caused by the antipsychotic agent such as acute dystonias, dyskinesias, akathesia and tremor may be reduced or prevented.

As used herein, the term “schizophrenic disorders” includes paranoid, disorganized, catatonic, undifferentiated and residual schizophrenia; schizophreniform disorder; schizoaffective disorder; delusional disorder; brief psychotic disorder; shared psychotic disorder; substance-induced psychotic disorder; and psychotic disorder not otherwise specified.

Other conditions commonly associated with schizophrenic disorders include self-injurious behavior (e.g. Lesch-Nyhan syndrome) and suicidal gestures.

Suitable antipsychotic agents of use in combination with a CB1 receptor modulator include the phenothiazine, thioxanthene, heterocyclic dibenzazepine, butyrophenone, diphenylbutylpiperidine and indolone classes of antipsychotic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. Suitable examples of dibenzazepines include clozapine and olanzapine. An example of a butyrophenone is haloperidol. An example of a diphenylbutylpiperidine is pimozide. An example of an indolone is molindolone. Other antipsychotic agents include loxapine, sulpiride and risperidone. It will be appreciated that the antipsychotic agents when used in combination with a CB1 receptor modulator may be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, olanzapine, haloperidol, pimozide and risperidone are commonly used in a non-salt form.

Other classes of antipsychotic agent of use in combination with a CB1 receptor modulator include dopamine receptor antagonists, especially D2, D3 and D4 dopamine receptor antagonists, and muscarinic m1 receptor agonists. An example of a D3 dopamine receptor antagonist is the compound PNU-99194A. An example of a D4 dopamine receptor antagonist is PNU-101387. An example of a muscarinic ml receptor agonist is xanomeline.

Another class of antipsychotic agent of use in combination with a CB1 receptor modulator is the 5-HT_{2A} receptor antagonists, examples of which include MDL100907 and fananserin. Also of use in combination with a CB1 receptor modulator are the serotonin dopamine antagonists (SDAs) which are believed to combine 5-HT_{2A} and dopamine receptor antagonist activity, examples of which include olanzapine and ziperasidone.

Still further, NK-1 receptor antagonists may be favorably employed with the CB1 receptor modulators of the present invention. NK-1 receptor antagonists for use in the present invention are selected from the classes of compounds described previously.

It will be appreciated that a combination of a conventional anti-asthmatic drug with a CB1 receptor modulator may provide an enhanced effect in the treatment of asthma, and may be used for the treatment or prevention of asthma, which method comprises administration to a patient in need of such treatment an amount of a compound of the present invention and an amount of an anti-asthmatic agent, such that together they give effective relief. Thus, according to a further aspect of the present invention there is provided the use of a CB1 receptor modulator and an anti-asthmatic agent for the manufacture of a medicament for the treatment or prevention of asthma.

Suitable anti-asthmatic agents of use in combination with a compound of the present invention include, but are not limited to: (a) VLA4 antagonists such as natalizumab and the compounds described in U.S. Pat. No. 5,510,332, WO97/03094, WO97/02289, WO96/40781, WO96/22966, WO96/20216, WO96/01644, WO96/06108, WO95/15973 and WO96/31206; (b) steroids and corticosteroids such as beclomethasone, methylprednisolone, betamethasone, prednisone, dexamethasone, and hydrocortisone; (c) antihistamines (H1-histamine antagonists) such as bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, desloratadine, cetirizine, fexofenadine, descarboethoxyloratadine, and the like; (d) non-steroidal anti-asthmatics including β2-agonists (such as terbutaline, metaproterenol, fenoterol, isoetharine, albuterol, bitolterol, salmeterol, epinephrine, and pirbuterol), theophylline, cromolyn sodium, atropine, ipratropium bromide, leukotriene antagonists (such as zafirlukast, montelukast, pranlukast, iralukast, pobilukast, and SKB-106,203), and leukotriene biosynthesis inhibitors (such as zileuton and BAY-1005); (e) anti-cholinergic agents including muscarinic antagonists (such as ipratropium bromide and atropine); and (f) antagonists of the chemokine receptors, especially CCR-3; and pharmaceutically acceptable salts thereof.

It will be appreciated that a combination of a conventional anti-constipation drug with a CB1 receptor modulator may provide an enhanced effect in the treatment of constipation, or chronic intestinal pseudo-obstruction, and for use for the manufacture of a medicament for the treatment or prevention of constipation or chronic intestinal pseudo obstruction.

The present invention also provides a method for the treatment or prevention of constipation, which method comprises administration to a patient in need of such treatment an amount of a compound of the present invention and an amount of an anti-constipation agent, such that together they give effective relief.

Suitable anti-constipation agents of use in combination with a compound of the present invention include, but are not limited to, osmotic agents, laxatives and detergent laxatives (or wetting agents), bulking agents, and stimulants; and pharmaceutically acceptable salts thereof. A class of osmotic agents can include, but is not limited to, sorbitol, lactulose, polyethylene glycol, magnesium, phosphate, sulfate, and pharmaceutically acceptable salts thereof. A class of laxatives and detergent laxatives, includes, but is not limited to, magnesium, docusate sodium, and pharmaceutically acceptable salts thereof. A class of bulking agents includes, but is not limited to, psyllium, methylcellulose, calcium polycarbophil, and pharmaceutically acceptable salts thereof. A class of stimulants includes, but is not limited to, anthroquinones, and phenolphthalein, and pharmaceutically acceptable salts thereof.

It will be appreciated that a combination of a conventional anti-cirrhosis drug with a CB1 receptor modulator may provide an enhanced effect in the treatment of cirrhosis of the liver, and for use for the manufacture of a medicament for the treatment or prevention of cirrhosis of the liver.

The present invention also provides a method for the treatment or prevention of cirrhosis of the liver, which method comprises administration to a patient in need of such treatment an amount of a compound of the present invention and an anti-cirrhosis agent, such that together they give effective relief.

Suitable anti-cirrhosis agents of use in combination with a compound of the present invention include, but are not limited to, corticosteroids, penicillamine, colchicine, interferon-γ, 2-oxoglutarate analogs, prostaglandin analogs, and other anti-inflammatory drugs and antimetabolites such as azathioprine, methotrexate, leflunamide, indomethacin, naproxen, and 6-mercaptopurine; and pharmaceutically acceptable salts thereof.

The method of treatment of this invention comprises a method of modulating the CB1 receptor and treating CB1 receptor mediated diseases by administering to a patient in need of such treatment a non-toxic therapeutically effective amount of a compound of this invention that selectively antagonizes the CB1 receptor in preference to the other CB or G-protein coupled receptors.

The term “therapeutically effective amount” means the amount the compound of structural formula I that will elicit the biological or medical response of a tissue, system, animal or human that is being sought by the researcher, veterinarian, medical doctor or other clinician, which includes alleviation of the symptoms of the disorder being treated. The novel methods of treatment of this invention are for disorders known to those skilled in the art. The term “mammal” includes humans, and companion animals such as dogs and cats.

The weight ratio of the compound of the Formula I to the second active ingredient may be varied and will depend upon the effective dose of each ingredient. Generally, an effective dose of each will be used. Thus, for example, when a compound of the Formula I is combined with a β-3 agonist the weight ratio of the compound of the Formula I to the β-3 agonist will generally range from about 1000:1 to about 1:1000, such as for example from about 200:1 to about 1:200. Combinations of a compound of the Formula I and other active ingredients will generally also be within the aforementioned range, but in each case, an effective dose of each active ingredient should be used.

Abbreviations used in the following Schemes and Examples: Ac: acetyl; aq.: aqueous; API-ES: atmospheric pressure ionization-electrospray (mass spectrum term); DEAD: diethyl azodicarboxylate; DMAP: 4-dimethylaminopyridine; DMF: dimethylformamide; DMSO: dimethylsulfoxide; EDC: 1-ethyl-3-(3-dimethylaminopropyl)-carbodiimide hydrochloride; EPA: ethylene polyacrylamide (a plastic); Et: ethyl; g: gram; h: hours; Hex: hexane; HOBt: 1-hydroxybenzotriazole; HPLC: high pressure liquid chromatography; HPLC/MS: high pressure liquid chromatography/mass spectrum; in vacuo: rotoevaporation; IPAC or IPAc: isopropyl acetate; KHMDS: potassium hexamethyldisilazide; LC: Liquid chromatography; LC/MS or LC-MS: liquid chromatography-mass spectrum; LDA: lithium diisopropyl amide; M: molar; MCPBA: 3-chloroperbenzoic acid; Me: methyl; MeOH: methanol; MHz: megahertz; min: minute; mL: milliliter; mmol: millimole; MS or ms: mass spectrum; N: normal; NaHMDS: sodium hexamethyldisilazide; NMR: nuclear magnetic resonance; PyBOP: (benzotriazol-1-yloxy)tripyrrolidinophosphonium hexafluorophosphate; Rₜ: retention time; rt or RT: room temperature; TFA: trifluoroacetic acid; THF: tetrahydrofuran; TLC: thin layer chromatography.

The compounds of this invention may be prepared by employing reactions as shown in the following schemes, in addition to other standard manipulations that are known in the literature or exemplified in the experimental procedures. The illustrative schemes below, therefore, are not limited by the compounds listed or by any particular substitutents employed for illustrative purposes. Substituent numbering as shown in the schemes does not necessarily correlate to that used in the claims and often, for clarity, a single substituent is shown attached to the compound in place of multiple substituents which are allowed under the definitions of Formula I defined previously.

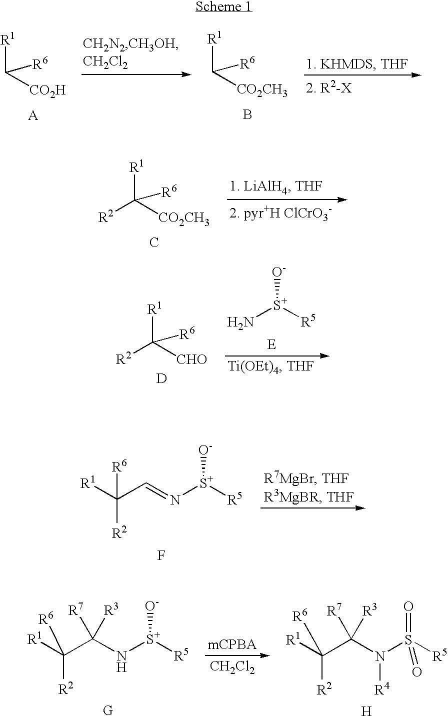

In Scheme 1, a substituted carboxylic acid A is converted to its methyl ester B which is subsequently alkylated with an appropriate halide in the presence of a strong base to afford ester C. The 15 ester is reduced and then oxidized to the aldehyde D. Reaction of D with the sulfinamide E will yield the imine F. A Grignard reagent is reacted with F to introduce R³ and R⁷ and afford sulfinamide G. Peracid oxidation of G will afford the sulfonamide H.

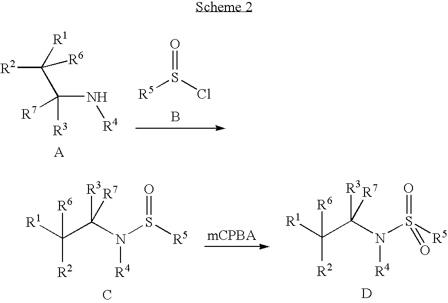

In Scheme 2, an appropriately substituted amine A is reacted with a sulfinyl chloride B in the presence of a hindered base to afford the sulfinamide C. Peracid oxidation of C will yield the sulfonamide D. This method is useful for the preparation of sterically hindered sulfonamides.

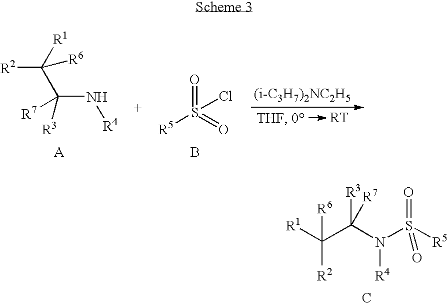

In Scheme 3, an appropriately substituted amine A is reacted with a sulfonyl chloride B in the presence of a hindered base to afford the sulfonamide C.

In order to illustrate the invention, the following examples are included. These examples do not limit the invention. They are only meant to suggest a method of reducing the invention to practice. Those skilled in the art may find other methods of practicing the invention which are readily apparent to them. However, those methods are also deemed to be within the scope of this invention.

General Procedures

The LC/MS analyses were preformed using a MICROMASS ZMD mass spectrometer coupled to an AGILENT 1100 Series HPLC utilizing a YMC ODS-A 4.6×50 mm column eluting at 2.5 mL/min with a solvent gradient of 10 to 95% B over 4.5 min, followed by 0.5 min at 95% B: solvent A=0.06% TFA in water; solvent B=0.05% TFA in acetonitrile. ¹H-NMR spectra were obtained on a 500 MHz VARIAN Spectrometer in CDCl₃ or CD₃OD as indicated and chemical shifts are reported as δ using the solvent peak as reference and coupling constants are reported in hertz (Hz).

### REFERENCE EXAMPLE 1

### N-[2,3-Bis(4-chlorophenyl)-1-methylpropyl]-amine hydrochloride

The preparation of the two diastereomers (alpha and beta) of N-[2,3-bis(4-chlorophenyl)-1-methylpropyl]-amine hydrochloride salt has been disclosed (Schultz, E. M, et al. J. Med Chem. 1967, 10, 717). Diastereomer α: LC-MS: calculated for C₁₆H₁₇Cl₂N 293. observed m/e 294 (M+H)⁺(Rₜ 2.5 min). Diastereomer β: LC-MS: calculated for C₁₆H₁₇Cl₂N 293. observed m/e 294 (M+H)⁺(Rₜ 2.2 min).

The amines of Reference Examples 2-9 were prepared by the same procedures described in Reference Example 1:

### REFERENCE EXAMPLE 2

### 2-Amino-3,4-diphenylbutane hydrochloride salt

Diastereomer α:

LC-MS: calculated for C₁₆H₁₉N 225. observed m/e 226 (M+H)⁺(2.0 min).

Diastereomer β:

LC-MS: calculated for C₁₆H₁₉N 225. observed m/e 226 (M+H)⁺(1.9 min).

### REFERENCE EXAMPLE 3

### 3-Amino-1,2-diphenylpentane hydrochloride salt

Diastereomer α:

LC-MS: calculated for C₁₇H₂₁N 239. observed m/e 240 (M+H)⁺(2.1 min).

Diastereomer β:

LC-MS: calculated for C₁₇H₂₁N 239. observed m/e 240 (M+H)⁺(2.0 min).

### REFERENCE EXAMPLE 4

### 1-Amino-1,2,3-triphenylpropane p-toluenesulfonate salt

Diastereomer α:

LC-MS: calculated for C₂₁H₂₁N 287. observed m/e 288 (M+H)⁺(2.3 min).

Diastereomer β:

LC-MS: calculated for C₂₁H₂₁N 287. observed m/e 288 (M+H)⁺(2.3 min).

### REFERENCE EXAMPLE 5

### 2-Amino-4-(4-chlorophenyl)-3-phenylbutane hydrochloride salt

Diastereomer α:

LC-MS: calculated for C₁₆H₁₈ClN 259. observed m/e 260 (M+H)⁺(2.3 min).

Diastereomer β:

LC-MS: calculated for C₁₆H₁₈ClN 259. observed m/e 260 (M+H)⁺(2.2 min).

### REFERENCE EXAMPLE 6

### 2-Amino-3-(4-chlorophenyl)4-phenylbutane hydrochloride salt

Diastereomer α:

LC-MS: calculated for C₁₆H₁₈ClN 259. observed m/e 260 (M+H)⁺(2.3 min).

Diastereomer β:

LC-MS: calculated for C₁₆H₁₈ClN 259. observed m/e 260 (M+H)⁺(2.1 min).

### REFERENCE EXAMPLE 7

### 2-Amino-4-(4-methoxycarbonylphenyl)-3-phenylbutane hydrochloride salt

Diastereomer α:

LC-MS: calculated for C₁₈H₂₁NO₂ 283. observed m/e 284 (M+H)⁺(2.0 min).

Diastereomer β:

LC-MS: calculated for C₁₈H₂₁NO₂ 283. observed m/e 284 (M+H)⁺(1.9 min).

### REFERENCE EXAMPLE 8

### 2-Amino-3-(2-Chlorophenyl)4-phenylbutane (mixture of diastereomers α/β 1:2)

LC-MS: calculated for C₁₆H₁₈ClN 259. observed m/e 260 (M+H)⁺(1.9/2.0 min).

### REFERENCE EXAMPLE 9

### 2-Amino-3-(4-methoxyphenyl)-4-phenylbutane (mixture of diastereomers α/β 2:5)

LC-MS: m/e 256 (M+H)⁺(1.7 min).

### REFERENCE EXAMPLE 10

### N-[3-(4-Chlorophenyl)-2-phenyl-1-methylpropyl]-amine hydrochloride (Diastereomer α)

### Step A: 3-4-Chlorophenyl)-2-phenylpropanoic acid, methyl ester

To a solution of methyl phenylacetate (12 g, 80 mmol) and 4-chlorobenzyl bromide (16 g, 80 mmol) in 250 mLanhydrous THF at −78° C. was added sodium hexamethyldisilazide (1 M in THF, 80 mL, 80 mmol) (potassium hexamethyldisilazide in toluene may be used with similar results). The reaction was allowed to warm to room temperature overnight. The volatile materials were removed on a rotary evaporator, and the resulting mixture was partitioned between saturated ammnonium chloride (200 mL) and EtOAc (200 mL). The organic layer was separated and the aqueous layer extracted with EtOAc (2×200 mL). The combined organic extracts were dried over anhydrous sodium sulfate, filtered, and concentrated to dryness to give the title compound.

¹H NMR (500 MHz, CD₃OD): δ 7.36-7.10 (m, 9H), 3.81 (dd, 1H), 3.52 (s, 3H), 3.36 (dd, 1H), 3.02 (dd, 1H).

### Step B: 3-(4-Chlorophenyl)-2-phenylpropanoic acid

To a mixture of methyl 3-4-chlorophenyl)-2-phenylpropionate (Step A, 20 g, 74 mmol) in acetonitrile (100 mL) and water (100 mL) was added lithium hydroxide monohydrate (8.8 g, 0.21 mol). After stirring at room temperature for 3 days, the volatile materials were removed by concentrating on a rotary evaporator and the residue was partitioned between water (300 mL) and hexane/ether (1:1, 200 mL). The water layer was separated, acidified to pH=2-3, and extracted with EtOAc (2×200 mL) The combined organic extracts were dried over anhydrous sodium sulfate, filtered, and concentrated to dryness to give the title compound. ¹H NMR (500 MHz, CD₃OD): δ 7.34-7.10 (m, 9H), 3.82 (dd, 1H), 3.36 (dd, 1H), 2.98 (dd, 1H).

### Step C: N-Methoxy-N-methyl-3-(4-chlorophenyl)-2-phenylpropanamide

To a solution of 3-(4-chlorophenyl)-2-phenylpropionic acid (Step B, 14 g, 55 mmol) in CH₂Cl₂ (125 mL) at 0° C. was added dimethyl formamide (50 μL) and oxalyl chloride (14 g, 0.11 mol) dropwise. The reaction was allowed to warm to room temperature overnight and concentrated to dryness to give the crude acyl chloride, which was used without further purification. Thus, to a solution of the acyl chloride in CH₂Cl₂ (250 mL) was added N-methoxy-N-methylamine hydrochloride (11 g, 0.11 mol) and triethyl amine (dried over activated molecular sieves, 30 mL, 0.22 mol) at 0° C. After stirring at room temperature for 4 h, the reaction mixture was diluted with ether (500 mL) and successively washed with water, dilute aqueous sodium hydrogen sulfate and brine, dried over anhydrous MgSO₄, filtered and concentrated to dryness to give the crude product, which was used without further purification. ¹H NMR (500 MHz, CD₃OD): δ 7.4-7.1 (m, 9H), 4.38 (br, 1H), 3.48 (s, 3H), 3.35 (dd, 1H), 3.10 (s, 3H), 2.92 (dd, 1H); LC-MS: m/e 304 (3.6 min).

### Step D: 4-(4-Chlorophenyl)-3-phenyl-2-butanone

To a solution of N-methoxy-N-methyl-3-(4-chlorophenyl)-2-phenylpropanamide (Step C, 16 g, 53 mmol, dried by azeotroping with toluene) in anhydrous THF (200 mL) at 0° C. was added methylmagnesium bromide (3 M in ether, 35 mL, 0.11 mol). After stirring at 0° C. for 2 h, the reaction was quenched with MeOH (5 mL) and 2 M hydrochloric acid (50 mL). The volatile materials were removed by concentrating on a rotary evaporator and the residue partitioned between saturated ammonium chloride (200 mL) and ether (200 mnL). The organic layer was separated, and the aqueous layer was extracted with ether (2×200 mL). The combined organic extracts were dried over anhydrous MgSO₄, filtered and concentrated to dryness to give the title compound, which was used without further purification. ¹H NMR (500 MHz, CD₃OD): δ 7.45-7.02 (m, 9H), 4.08 (dd, 1H), 3.34 (dd, 1H), 2.90 (dd, 1H), 2.03 (s, 3H).

### Step E: 4-(4Chlorophenyl)-3-phenyl-2-butanol

To a solution of 4-(4-chlorophenyl)-3-phenyl-2-butanone (Step D, 13 g, 50 mmol) in MeOH (100 mL) at 0° C. was added sodium borohydride (3.8 g, 100 nmmol). After stirring at 0° C. for 30 min, the reaction was quenched by addition of 2 M hydrochloric acid (50 mL). The volatile materials were removed by concentrating on a rotary evaporator and the residue partitioned between water (100 mL) and EtOAc (200 mL). The organic layer was separated and the aqueous layer extracted with EtOAc (2×200 mL). The combined organic extracts were washed with brine, dried over anhydrous sodium sulfate, filtered and concentrated to dryness to give the crude product, which was purified by flash column chromatography on silica gel eluted with 10% EtOAc in hexane to afford the pure faster eluting isomer and a mixture containing both the faster eluting isomer and the slower eluting isomer. Faster eluting isomer: ¹H NMR (500 MHz, CD₃OD): δ 7.25-7.00 (m, 9H), 4.00 (m, 1H), 3.15 (m, 1H), 2.97 (m, 1H), 2.85 (m, 1H), 1.10 (d, 3H).

### Step F: 4-(4-Chlorophenyl)-2-methanesulfonyloxy-3-phenylbutane

To a solution of 4-4-chlorophenyl)-3-phenyl-2-butanol (Step E, faster eluting isomer, 9.0 g, 34 mmol) in EtOAc (100 mL) at 0° C. was added triethyl amine (dried over activated molecular sieves, 5.8 mL. 42 mmol) and methanesulfonyl chloride (3.0 mL, 38 mmol). After stirring at 0° C. for 30 min, the reaction was quenched by addition of saturated aqueous sodium bicarbonate (100 mL). After stirring at room temperature for 1 h, the organic layer was separated, dried over anhydrous sodium sulfate, filtered, and concentrated to dryness to give the title compound, which was used without further purification. ¹H NMR (500 MHz, CD₃OD): δ 7.3-7.0 (m, 9H), 5.05 (m, 1H), 3.2-3.0 (m, 3H), 2.80 (s, 3H), 1.40 (d, 3H).

### Step G: 2-Azido-4-(4-chlorophenyl)-3-phenylbutane

To a solution of 4-4-chlorophenyl)-2-methanesulfonyloxy-3-phenylbutane (Step F, 12 g, 34 mmol) in DMF (50 mL) was added sodium azide (11 g, 0.17 mol). After stirring at 120° C. for 1 h, the reaction mixture was poured into water (200 mL), and the product was extracted with ether (2×100 mL). The combined organic extracts were washed with water, dried over MgSO₄, filtered and concentrated to dryness, and the residue was purified on a silica gel column eluting with hexane to give the title compound.

### Step H: 2-(N-tert-Butoxycarbonyl)amino-4-(4-chlorophenyl)-3-phenylbutane

To a solution of 2-azido-4-(4-chlorophenyl)-3-phenylbutane (Step G, 7.0 g, 24 mmol) in EtOAc (150 mL) was added di(tert-butyl) dicarbonate (8.0 g, 37 mmol) and platinum dioxide (0.50 g, 2.2 mmol). The mixture was degassed and filled with hydrogen with a balloon. After stirring for 1 day, the reaction mixture was filtered through CELITE diatomaceous earth, and the filtrate was concentrated to give the crude product, which was contaminated with some unreacted di(tert-butyl) dicarbonate. ¹H NMR (500 MHz, CD₃OD): δ 7.25-6.88 (m, 9H), 3.89 (m, 1H), 3.20 (m, 1H), 2.86-2.77 (m, 2H), 1.54 (s, 9H), 0.92 (d, 3H).

### Step I: N-[3-(4-Chlorophenyl)-2-phenyl-1-methylpropyl]-amine hydrochloride (Diastereomer α)

2-(N-tert-butoxycarbonyl)amino-4-(4-chlorophenyl)-3-phenylbutane (Step H, 7.0 g, 24 mmol) was treated with a saturated solution of hydrogen chloride in EtOAc (100 mL) at room temperature for 30 min (4 M hydrogen chloride in dioxane may be used with similar results). The mixture was concentrated to dryness to give the title compound. ¹H NMR (500 MHz, CD₃OD): δ 7.35-6.98 (m, 9H), 3.62 (m, 1H), 3.20 (dd, 1H), 3.05 (m, 1H), 2.98 (dd, 1H), 1.19 (d, 3H). LC-MS: m/e 260 (M+H)⁺(2.3 min).

### REFERENCE EXAMPLE 11

### N-[3-(4-Chlorophenyl)-2(S)-phenyl-1(S)-methylpropyl]-amine hydrochloride

### Step A: 4-(4-Chlorophenyl)-3(S)-phenyl-2(R)-butanol

A sample of magnesium (20 g, 0.82 mol) was activated by stirring under nitrogen for 12 h, and anhydrous ether (100 mL) was added to cover the solid material. The mixture was cooled to 0° C., and was added 4-chlorobenzyl chloride (40 g, 0.25 mmol) in 400 mL anhydrous ether dropwise. After stirring at room temperature for 1 h, a sample of the above solution (32 mL) was added to (1R,2R)-1-phenylpropylene oxide (1.0 g, 7.5 mmol) in 100 mL ether at 0° C. via syringe. After stirring at 0° C. for 2 h, the reaction was quenched by addition of saturated aqueous ammonium chloride (100 mL). The organic layer was separated and the aqueous layer extracted with ether (2×100 mL). The combined organic extracts were washed with brine, dried over anhydrous MgSO₄, filtered, and concentrated to dryness, and the residue was purified by flash column chromatography on silica gel eluted with hexane to 15% EtOAc in hexane to afford the title compound. ¹H NMR (500 MHz, CD₃OD): δ 7.28-7.02 (m, 9H), 4.01 (m, 1H), 3.14 (dd, 1H), 2.97 (dd, 1H), 2.85 (m, 1H), 1.12 (d, 3H).

### Step B: N-[3-(4-chlorophenyl)-2(S)-phenyl-1(S)-methylpropyl]-amine, hydrochloride

The product of Step A (4-(4-chlorophenyl)-3(S)-phenyl-2(R)-butanol, 1.8 g, 7.0 mmol) was converted to the title compound following the steps described in Reference Example 10, Steps F-I, except hydrogen chloride in dioxane (4 M) was used in place of hydrogen chloride in EtOAc. ¹H NMR (500 MHz, CD₃OD): δ 7.35-6.98 (m, 9H), 3.62 (m, 1H), 3.20 (dd, 1H), 3.05 (m, 1H), 2.98 (dd, 1H), 1.19 (d, 3H). LC-MS: m/e 260 (M+H)⁺(2.3 min).

### REFERENCE EXAMPLE 12

### 2-Amino-4-(4-chlorophenyl)-3-(3-fluorophenyl)butane hydrochloride salt (mixture of diastereomers α/β 5:1)

### Step A: Methyl 3-(4-Chlorophenyl)-2-(3-flurophenyl)propionate

To a solution of 3-fluorophenylacetic acid (5.0 g, 32 mmol) in MeOH (25 mL) and CH₂Cl₂ (25 mL) at 0° C. was added trimethylsilyldiazomethane (2 M in hexane, 30 mL, 60 mmol). After stirring at room temperature for 15 min, the reaction mixture was concentrated to dryness, and the residue was azeotroped with toluene to give the crude methyl 3-fluorophenylacetate (5.6 g), which was used without further purification. Thus, the crude methyl 3-fluorophenylacetate obtained above (2.5 g, 15 mmol) was converted to the title compound (purified on silica gel) by reacting with 4-chlorobenzyl bromide (4.6 g, 22 nmmol) and sodium hexamethyldisilazide (1 M in THF, 15 mL, 15 mmol) following the procedure described in Reference Example 10, Step A. ¹H NMR (400 MHz, CD₃OD): δ 7.35-6.88 (m, 8H), 3.92 (t, 1H), 3.60 (s, 3H), 3.34 (dd, 1H), 3.00 (dd, 1H). LC-MS: m/e 305 (M+Na)⁺(3.9 min).

### Step B: N-Methoxy-N-methyl-3-4-chlorophenyl)-2-(3-fluororophenyl)propanamide

To a suspension N-methoxy-N-methylamine hydrochloride (2.0 g, 21 mmol) in 50 mL CH₂Cl₂ at 0° C. was added dimethylaluminum chloride (1 M in hexane, 21 mL, 21 mmol). After stirring at room temperature for 1 h, a solution of methyl 3-(4-chlorophenyl)-2-(3-flurophenyl)propionate (Step A, 2.0 g, 10 mmol) in CH₂Cl₂ (10 mL) was added, and the resulting mixture was stirred overnight. The reaction mixture was quenched by addition of MeOH (5 mL), and the resulting mixture was concentrated with silica gel (50 g). The material was loaded onto a silica gel column, which was eluted with 10% EtOAc in hexane to 2% ammonia in MeOH (2 M) in 10% EtOAc/hexane to give the title compound. ¹H NMR (400 MHz, CD₃OD): δ 7.35-6.90 (m, 8H), 4.39 (br, 1H), 3.41 (s, 3H), 3.38-3.30 (m, 1H), 3.08 (s, 3H), 2.92 (dd, 1H). LC-MS: m/e 322 (M+H)⁺(3.6 min).

### Step C: 4-(4-Chlorophenyl)-3-(3-fluorophenyl)-2-butanol

The product of Step B (N-methoxy-N-methyl-3-4-chlorophenyl)-2-phenylpropionamide) (0.74 g, 2.3 mmol) was converted to the title compound (a 5:1 mixture of diastereomers) following the procedure described in Reference Example 10, Steps D-E. ¹H NMR (400 MHz, CD₃OD): δ 7.22-6.78 (m, 8H), 3.98 (m, 1H), 3.11 (dd, 1H), 2.94 (dd, 1H), 2.85 (m, 1H), 1.08 (d, 3H).

### Step D: 2-Azido-4-(4-chlorophenyl)-3-(3-fluorophenyl)butane

To a mixture of 4-(4-chlorophenyl)-2-(3-fluorophenyl)-2-butanol (Step C, 0.65 g, 2.3 mmol), triphenylphosphine (1.2 g, 4.7 mmol), imidazole (0.32 g, 4.7 mmol) and zinc azide dipyridine complex (Viaud, M. C.; Rollin, P. Synthesis 1990, 130) (0.72 g, 2.3 mmol) in 10 mL CH₂Cl₂ was added diethylazodicarboxylate (0.73 mL, 4.7 mmol) at 0° C. After stirring at room temperature for 30 min, the resulting mixture was concentrated with silica gel (20 g) and loaded onto a silica gel column, which was eluted with 2% ether in hexane to 2% ammonia in MeOH (2 M) in 2% ether/hexane to give the title compound. ¹H NMR (400 MHz, CD₃OD): δ 7.25-6.85 (m, 8H), 3.76 (m, 1H), 3.33 (m, 1H), 2.92 (m, 2H), 1.15 (d, 3H).

### Step E: 2-Amino-4-(4-Chlorophenyl)-3-(3-fluorophenyl)butane hydrochloride salt (mixture of diastereomers α/β 5:1)

The product of Step D (2-azido-4-(4-chlorophenyl)-3-3-fluorophenyl)butane) (0.49 g, 1.6 mmol) was converted to the title compound following the steps described in Reference Example 10, Steps H-I. ¹H NMR (400 MHz, CD₃OD): δ 7.32-6.90 (m, 7H), 3.61 (m, 1H), 3.20 (dd, 1H), 3.11 (m, 1H), 2.92 (dd, 1H), 1.19 (d, 3H). LC-MS: m/e 278 (M+H)⁺(2.4 min).

The amines of Reference Examples 13-16 were prepared according to the procedures described in Reference Example 12:

### REFERENCE EXAMPLE 13

### 2-Amino-4-(4-chlorophenyl)-3-(2-fluorophenyl)butane hydrochloride salt (mixture of diastereomers α/β 10:1)

LC-MS: m/e 278 (M+H)⁺(2.3 min).

### REFERENCE EXAMPLE 14

### 2-Amino-4-(4-chlorophenyl)-3-(4-fluorophenyl)butane hydrochloride salt (mixture of diastereomers α/β 10:1)

LC-MS: m/e 278 (M+H)⁺(2.5 min).

### REFERENCE EXAMPLE 15

### 2-Amino-4-(4-chlorophenyl)-3-(2-pyridyl)butane hydrochloride salt (mixture of diastereomers α/β 10:1)

LC-MS: m/e 261 (M+H)⁺(1.6 min).

### REFERENCE EXAMPLE 16

### 2-Amino-4-(4-chlorophenyl)-3-(4-pyridyl)butane hydrochloride salt (mixture of diastereomers α/β 10:1)

Trimethyl aluminum was used in place of dimethylaluminum chloride at Step B of Reference Example 12. LC-MS: m/e 261 (M+H)⁺.

### REFERENCE EXAMPLE 17

### 2-Amino-4-(4-cyanophenyl)-3-phenylbutane hydrochloride salt (mixture of diastereomers α/β 10:1)

### Step A: 4-(4-Cyanophenyl)-3-phenyl-2-butanone

To a solution of phenylacetone (1.2 g, 9.0 mmol) and 4-cyanobenzyl chloride (1.4 g, 9.0 mmol) in 20 mL CH₂Cl₂at −78° C. was added cesium hydroxide monohydrate (4.5 g, 27 mmol) and tetrabutyl ammonium iodide (20 mg). The reaction was allowed to warm to room temperature over 6 h, and the resulting mixture partitioned between brine (100 mL) and EtOAc (100 mL). The organic layer was separated and the aqueous layer extracted with EtOAc (2×100 mL). The combined organic extracts were dried over MgSO₄, filtered, and concentrated to dryness, and the residue was purified by flash column chromatography on silica gel eluted with 20-50% EtOAc in hexane to give the title compound. ¹H NMR (500 MHz, CD₃OD): δ 7.52 (d, 2H), 7.34-7.16 (m, 7H), 4.12 (dd, 1H), 3.41 (dd, 1H), 3.00 (dd, 1H). LC-MS: m/e 250 (M+H)⁺(3.2 min).

### Step B: 2-Amino-4-(3-cyanophenyl)-3-phenylbutane hydrochloride salt (mixture of diastereomers α/β 10: 1)

The product of Step A (4-(4-cyanophenyl)-3-phenyl-2-butanone) (1.0 g, 4.0 mmol) was converted to the title compound following the procedure described in Reference Example 10, Steps E-I. LC-MS: m/e 251 (M+H)⁺(1.9 min).

### REFERENCE EXAMPLE 18

### 2-Amino-4-(5chloro-2-pyridyl)-3-phenylbutane hydrochloride salt (mixture of diastereomers α/β 10:1)

5-Chloro-2-choromethylpyridine (Weidmann, K. et al. J. Med. Chem. 1992, 35, 438) was used in place of 4-cyanobenzyl bromide in Step A of Reference Example 17. LC-MS: m/e 261 (M+H)⁺.

### REFERENCE EXAMPLE 19

### N-[3-(4-chlorophenyl)-2-(3-pyridyl)-1-methylpropyl]-amine, hydrochloride (mixture of diastereomers α/β 10:1)

### Step A: 4-(4-Chlorophenyl)-3-pyridyl-2-butanone

To a solution of 3-pyridylacetone hydrochloride (Wibaud, van der V. Recl. Trav. Chim. Pays-Bas. 1952, 71, 798) (10 g, 58 mmol) and 4-chlorobenzyl chloride (9.1 g, 58 mmol) in 100 mL CH₂Cl₂at −78° C. was added cesium hydroxide monohydrate (39 g, 0.23 mol) and tetrabutyl ammonium iodide (1 g). The reaction was allowed to warm to room temperature overnight, and the resulting mixture was partitioned between brine (100 mL) and EtOAc (100 mL). The organic layer was separated and the aqueous layer extracted with EtOAc (2×100 mL). The combined organic extracts were dried over anhydrous MgSO₄, filtered, and concentrated to dryness to give the title compound. ¹H NMR (500 MHz, CD₃OD): δ 8.42 (d, 1H), 8.34 (d, 1H), 7.72 (d, 1H), 7.40 (dd, 1H), 7.18 (d, 2H), 7.06 (d, 1H), 4.23 (dd, 1H), 3.38 (dd, 1H), 2.95 (dd, 1H), 2.10 (s, 3H). LC-MS: m/e 260 (M+H)⁺(1.9 min).

### Step B: N-[3-(4-chlorophenyl)-2-(3-pyridyl)-1-methylpropyl]-amine, hydrochloride (mixture of diastereomers α/β 10:1)

The product of Step A (4-(4-chlorophenyl)-3-pyridyl-2-butanone) (14 g, 57 mmol) was converted to the title compound following the procedure described in Reference Example 10, Steps E-I. LC-MS: m/e 261 (M+H)⁺(1.2 min).

### REFERENCE EXAMPLE 20

### 2-Amino-4-(2,4-dichlorophenyl)-3-(4-chlorophenyl)butane hydrochloride salt (3 isomers)

### Step A: Methyl 3-(2,4-Dichlorophenyl)-2-(4-chorophenyl)propionate

A sample of 4-chlorophenylacetic acid (4.2 g, 25 mmol) was converted to the title compound (6.5 g) following the procedure in Reference Example 12, Step A substituting 4-chlorophenylacetic acid for 3-fluorophenylacetic acid and 2,4-dichlorobenzyl bromide for 4-chlorobenzyl bromide following the procedures described in Reference Example 10, Step A. ¹H NMR (500 MHz, CD₃OD): δ 7.40 (d, 1H), 7.32-7.22 (m, 4 H), 7.15 (dd, 1H), 7.08 (d, 1H), 4.00 (t, 1H), 3.62 (s, 3H), 3.44 (dd, 1H), 3.12 (dd, 1H).

### Step B: 3-(2,4-Dichlorophenyl)-2-(4-chlorophenyl)propanol

To a solution of methyl 3-(2,4-dichlorophenyl)-2-(4-chorophenyl) propionate (6.4 g, 8.6 mmol) in 50 mL ether at −40° C. was added lithium aluminum hydride (1.4 g, 37 mmol), and the reaction was allowed to warm to room temperature over 2 h. The reaction was quenched by addition of MeOH (3 mL) dropwise at −10° C., and the mixture was partitioned between 100 mL saturated ammonium chloride and EtOAc (100 mL). The organic layer was separated and the aqueous layer extracted with EtOAc (2×100 mL). The combined organic extracts were dried over anhydrous MgSO₄, filtered, and concentrated to dryness to give the title compound, which was used without further purification. ¹H NMR (400 MHz, CD₃OD): δ 7.4-6.9 (m, 7H), 3.72 (m, 2H), 3.24 (dd, 1H), 3.16 (m, 1H), 2.85 (dd, 1H).

### Step C: 3-(2,4-Dichlorophenyl)-2-(4-chorophenyl)propanal

To a solution of 3-(2,4-dichlorophenyl)-2-(4-chorophenyl)propanol (Step B, 0.89 g, 2.8 mmol) in 20 mL CH₂Cl₂ was added crushed activated molecular sieves (4 g). After stirring at room temperature for 10 min, pyridinium chlorochromate (0.90 g, 4.2 mmol) was added. After stirring at room temperature for 1 h, CELITE diatomaceous earth (4 g) was added followed by 100 mL ether. The resulting mixture was filtered through a silica gel pad, which was washed with ether (2×50 mL). The filtrate was concentrated to dryness and azeotroped with toluene to give the title compound, which was used without further purification.

### Step D: N-[3-(2,4-Dichlorophenyl)-2-(4-chorophenyl)propylidene]-2-methylpropanesulfinamide

To a solution of 3-(2,4-dichlorophenyl)-2-4-chorophenyl)propanal (Step C, 0.90 g, 2.8 mmol) in 6 mLTHF was added (R)-(+)-2-methyl-2-propane-sulfinamide (0.5 gm, 4.1 mmol) followed by the addition of titanium tetraethoxide (1.5 nL, 8.0 mmol). After stirring at room temperature overnight, the reaction mixture was added to a well-stirred brine solution (50 mL). The resulting mixture was filtered through CELITE diatomaceous earth and washed with EtOAc (20 mL), and the filtrate was extracted with EtOAc (2×50 mL). The combined extracts were dried over anhydrous sodium sulfate, filtered, and concentrated to dryness, and the residue was purified by flash column chromatography on silica gel eluted with 10% ether in hexane to give the title compound as a 1:1 Imixture of diastereomers. ¹H NMR (500 MHz, CD₃OD): δ 8.11 (m, 1H), 7.41 (m, 1H), 7.35-7.31 (m, 4 H), 7.16-7.06 (m, 2H), 4.26 (m, 1H), 3.78-3.58 (m, 1H), 3.22-3.14 (m, 1H), 1.13/1.12 (s, 9H).

### Step E: N-[3-(2,4-Dichlorophenyl)-2-(4-chorophenyl)-1-methylpropyl]-2-methylpropanesulfinamde (3 isomers)

To a solution of N-[3-(2,4-dichlorophenyl)-2-(4-chorophenyl)-1-methylpropylidene]-2-methylpropanesulfinamde (Step D, 0.51 g, 1.3 mmol) in 6 mL CH₂Cl₂ at −60° C. was added methylmagnesium bromide (3 M in ether, 0.90 mL, 2.7 mmol). After stirring at −60° C. for 6 h, the reaction was allowed to warm to room temperature overnight. The resulting mixture was partitioned between saturated aqueous ammonium chloride (50 mL) and EtOAc (50 mL). The organic layer was separated and the aqueous layer extracted with EtOAc (2×50 mL). The combined extracts were dried over anhydrous sodium sulfate, filtered, and concentrated to dryness, and the residue was purified by flash column chromatography on silica gel eluted with 30 to 50% EtOAc in hexane to give the title compound as one pure faster eluting enantiomer and a 1:1 mixture of slower co-eluting diastereomers. The addition of the methyl Grignard reagent was apparently stereoselective for one of the sulfinamide diastereomers.

Faster eluting isomer: ¹H NMR (500 MHz, CD₃OD): δ 7.30 (d, 1H), 7.22 (d, 2H), 7.12 (d, 2H), 7.03 (dd, 1H), 6.94 (d, 1H), 3.62 (m, 1H), 3.56 (dd, 1H), 2.97 (dd, 1H), 1.23 (s, 9H), 1.04 (d, 3H). LC-MS: m/e 432 (M+H)⁺(4.2 min). Slower eluting isomers (1:1): ¹H NMR (500 MHz, CD₃OD): δ 7.33/7.30 (d, 1H), 7.21/7.18 (d, 2H), 7.06/7.04 (d, 2H), 6.99/6.97 (dd, 1H), 6.79/6.75 (d, 1H), 3.70-3.55 (m, 1H), 3.07/2.97 (m, 1H), 2.90/2.80 (dd, 1H), 1.32/0.95 (s, 9H), 1.49/1.10 (d, 3H).

### Step F: 2-Amino-4-2,4-dichlorophenyl)-3-(4-chorophenyl)butane hydrochloride (3 isomers)

To a solution of N-[3-(2,4-dichlorophenyl)-2-(4-chorophenyl)-1-methylpropyl]-2-methylpropanesulfinamde (Step F, faster eluting isomer, 50 mg, 0.11 mmol) in 5 mL MeOH was added hydrogen chloride in dioxane (4 M, 2 mL). After stirring at room temperature for 10 min, the reaction mixture was concentrated to dryness to give the title compound as one pure isomer.

Isomer 1: ¹H NMR (500 MHz, CD₃OD): δ 7.35 (d, 1H), 7.29 (d, 2H), 7.15 (d, 2H), 7.06 (dd, 1H), 6.91 (d, 1H), 3.68 (m, 1iH), 3.36 (dd, 1H), 3.06 (dd, 1H), 1.18 (d, 3H).

LC-MS: m/e 328 (M+H)⁺(2.8 min). The two slower co-eluting isomers were treated in the same fashion to give two other isomers of the title compound. Isomer 2 and 3 (1:1): LC-MS: m/e 328 (M+H)⁺(2.7/2.8 min).

### REFERENCE EXAMPLE 21

### 2-Amino-4-(4-chloro-2-fluorophenyl)-3-(4-chlorophenyl)butane hydrochloride salt (Isomers, 1, 2 and 3)

The title compound was prepared according to the procedures of Reference Example 20 substituting 2,5-dichlorobenzyl bromide with 4-chloro-2-fluorobenzyl bromide.

Isomer 1: LC-MS: m/e 312 (M+H)⁺(2.6 min).

Isomer 2 and 3 (1:1): LC-MS: m/e 312 (M+H)⁺(2.5/2.6 min).

### REFERENCE EXAMPLE 22

### 2-(4-Chlorophenyloxy)-2-(4chlorophenyl)ethylamine hydrochloride salt

### Step A: 2-(4-Chlorophenyloxy)-2-(4-chlorophenyl)ethanol

To a suspension of 2-(4-chlorophenyloxy)-2-(4-chlorophenyl)acetic acid (Newman et al J. Amer. Chem. Soc. 1947, 69, 718) (1.0 g, 3.4 nmmol) in 10 mLTHEF at 0° C. was added borane (1 M in THF, 6.8 mL, 6.8 mmol). After stirring at room temperature for 2 h, the reaction was quenched by addition of 2 M hydrochloric acid (10 mL). The volatile materials were removed on a rotary evaporator, and the resulting mixture was partitioned between brine (20 mL) and EtOAc (30 mL). The organic layer was separated and the aqueous layer extracted with EtOAc (2×20 mL). The combined extracts were dried over anhydrous sodium sulfate, filtered, and concentrated to dryness to give the title compound, which was used without further purification. LC-MS: m/e 283 (M+H)⁺(3.4 min).

### Step B: 2-(4-Chlorophenoylxy)-2-(4chlorophenyl)ethyl Azide

2-4-Chlorophenyloxy)-2-(4-chlorophenyl)ethanol (Step A, 0.45 g, 2.4 mmol) was converted to the title compound (0.29 g) following the procedure described in Reference Example 12, Step D. ¹H NMR (500 MHz, CD₃OD): δ 7.41 (d, 2H), 7.37 (d, 2H), 7.18 (d, 2H), 6.86 (d, 2H), 5.42 (dd, 1H), 3.69 (dd, 1H), 3.45 (dd, 1H). LC-MS: m/e 308 (M+H)⁺(4.3 min).

### Step C: 2-(4-Chlorophenoylxy)-2-(4-chlorophenyl)ethylamine

To a solution of 2-(4-chlorophenoylxy)-2-(4-chlorophenyl)ethyl azide (Step B, 0.23 g, 0.75 mmol) in 4 mLTHF at −20° C. was added trimethylphosphine (0.18 mL, 1.8 mmol), and the reaction was allowed to warm to room temperature over 2 h. Lithium hydroxide monohydrate (61 mg, 1.5 mmol) was added followed by 2 mL water. After stirring at room temperature for 30 min, the reaction was quenched by addition of 2 M hydrochloric acid (final pH=2). The volatile materials were removed on a rotary evaporator, and the resulting mixture was partitioned between brine (20 mL), 5 N aqueous sodium hydroxide (20 mL), ether (20 mL) and toluene (20 mL). The organic layer was separated and the aqueous layer extracted with ether (40 mL). The combined extracts were dried over anhydrous MgSO₄, filtered, and concentrated to dryness to give the title compound (0.43 g), which was contaminated with trimethylphosphine oxide and was used without further purification. ¹H NMR (500 MHz, CD₃OD): δ 7.46-7.40 (m, 4H), 7.20 (d, 2H), 6.91 (d, 2H), 5.53 (m, 2H), 3.36 (m, 2H). LC-MS: m/e 282 (M+H)⁺ (2.5 min).

### REFERENCE EXAMPLE 23

### 2,2-Bis(4-chlorophenyl)ethylamine hydrochloride salt

### Step A: Methyl 3,3-Bis(4-chlorophenyl)propenoate

A mixture of di(4-chlorophenyl)ketone (7.5 g, 30 mmol) and methyl (triphenylphosphoranylidene)acetate (10 g, 30 mmol) in 20 mL toluene was heated at 130° C. while allowing the solvent to slowly evaporate overnight. The resulting mixture was dissolved in CH₂Cl₂ (20 mL) and toluene (20 mL) and was concentrated with 30 g silica gel. The material was loaded onto a silica gel column, which was eluted with 6:3:1 hexane/CH₂Cl₂/ether to give the title compound.

### Step B: Methyl 3,3-Bis(4-chlorophenyl)propionate

A suspension of methyl 3,3-bis(4-chlorophenyl)propenoate (Step A, 3.0 g, 14 mmol) and platinum dioxide (0.30 g) in MeOH (20 mL) and 2 M aqueous hydrochloric acid (1 mL) was degassed and filled with hydrogen with a balloon. After stirring at room temperature for 2 h, the reaction mixture was filtered through CELITE diatomaceous earth, and the filtrate was concentrated to dryness. The residue was dissolved in 50 mL ether and was concentrated with 20 g silica gel. The material was loaded onto a silica gel column, which was eluted with 10% ether in hexane to give the title compound. ¹H NMR (500 MHz, CD₃OD): δ 7.29-7.22 (m, 4H), 4.50 (t, 1H), 3.56 (s, 3H), 3.07 (d, 2H). LC-MS: m/e 309 (M+H)⁺(4.1 min).

### Step C: 3,3-Bis(4-chlorophenyl)propionic Acid

A mixture of methyl 3,3-bis(4-chlorophenyl)propionate (Step B, 0.78 g, 3.9 mmol), lithium hydroxide monohydrate (0.33 g, 7.8 mmol) in 1:1:1 MeOH/THF/water (15 mL) was stirred at room temperature overnight. The resulting mixture was partitioned between 2 M aqueous hydrochloric acid (50 mL) and ether (50 mL). The organic layer was separated and the aqueous layer extracted with EtOAc (2×50 mL). The combined extracts were dried over anhydrous MgSO₄, filtered, and concentrated to dryness to give the title compound. ¹H NMR (500 MHz, CD₃OD): δ 7.29-7.23 (m, 4H), 4.49 (t, 1H), 3.02 (d, 2H).

### Step D: N-[2,2-Bis(4-chlorophenyl)ethyl]allylcarbamate

To a solution of 3,3-bis(4-chlorophenyl)propionic acid (Step C, 0.32 g, 1.1 mmol) and triethyl amine (0.60 mL, 4.3 mmol) in 4 mLTHF at 0° C. was added ethyl chloroformate (0.31 mL, 3.3 mmol). After stirring at room temperature for 30 min, the reaction was cooled to 0° C., and was added sodium azide (0.35 g, 5.4 mmol) in 2 mLwater. After stirring at room temperature for 1 h, the reaction mixture was partitioned between brine (20 mL) and EtOAc (20 mL). The organic layer was separated and the aqueous layer extracted with EtOAc (2×20 mL). The combined extracts were dried over anhydrous sodium sulfate, filtered, and concentrated to dryness, and the residue was dissolved in allylic alcohol (1 mL) and toluene (1 mL). After stirring at 80° C. overnight, the reaction mixture was concentrated to dryness, and the residue was purified by flash column chromatography on silica gel column eluted with 20% EtOAc in hexane to give the title compound. ¹H NMR (500 MHz, CD₃OD): δ 7.30-7.21 (m, 4H), 5.84 (m, 1H), 5.17 (dd, 1H), 5.10 (dd, 1H), 4.46 (d, 2H), 4.22 (t, 1H), 3.68 (d, 2H). LC-MS: m/e 350 (M+H)⁺(3.9 min).

### Step E: 2,2-Bis(4-chlorophenyl)ethylamine hydrochloride salt

To a solution of N-[2,2-bis(4-chlorophenyl)ethyl]allylcarbamate (Step D, 0.26 g, 0.73 mmol) in 1.5 MLTHF at 0° C. was added tetrakis (triphenylphosphine)palladium (85 mg, 0.073 mmol) and triphenylsilane (0.18 mL, 1.1 mmol). After stirring at 0° C. for 1 h, the reaction mixture was partitioned between ether (20 mL) and 2 M hydrochloric acid (20 mL). The aqueous layer was separated, and was added 5 N aqueous sodium hydroxide (final pH>12). The product was extracted with ether (3×30 mL), and the combined extracts were dried over sodium hydroxide, and filtered through CELITE, diatomaceous earth. After addition of 4 M hydrogen chloride in dioxane (2 mL), the filtrate was concentrated to dryness to give the title compound. ¹H NMR (500 MHz, CD₃OD): δ 7.40-7.34 (m, 4H), 4.28 (m, 1H), 3.62 (d, 2H). LC-MS: m/e 266 (M+H)⁺(2.3 min).

### REFERENCE EXAMPLE 24

### 2-Amino-3-(4-chlorophenylthio)-3-(4-chlorophenyl)propane hydrochloride salt (two diastereomers)

### Step A: Methyl 2-(4-Chlorophenylthio)-2-4-chlorophenyl)acetate

To a solution of 2-4-chlorophenylthio)-2-(4-chlorophenyl)acetic acid (Nicolaescu et al Rev. Roum. Chim. 1979, 24, 137) (1.0 g, 3.0 mmol) in MeOH (10 mL) and CH₂Cl₂ (10 mL) at 0° C. was added trimethylsilyldiazomethane (2 M in hexane) until a yellow color persisted. Concentration afforded the title compound, which was used without further purification.

### Step B: 2-Amino-3-(4-chlorophenylthio)-3-(4-chlorophenyl)propane hydrochloride salt (two diastereomers)

The product of Step A (methyl 2-(4-chlorophenylthio)-2-(4-chlorophenyl)acetate) (1.1 g, 3.0 mmol) was converted to the title compound following the procedures described in Reference Example 12, Steps B-E. LC-MS: m/e 312 (M+H)⁺(2.7 min).

### REFERENCE EXAMPLE 25

### 2-Amino-3,4-bis(4-chlorophenyl)-2-methylbutane hydrochloride salt

### Step A: Methyl 2,3-Bis(4-chlorophenyl)propionate

The title compound was prepared following the procedure described in Reference Example 10, Step A, substituting methyl phenylacetate with methyl 4-chlorophenylacetate. ¹H NMR (500 MHz, CD₃OD): δ 7.30-7.22 (m, 4H), 7.19 (d, 2H), 7.09 (d, 2H), 3.90 (t, 1H), 3.58 (s, 3H), 3.32 (dd, 1H), 2.98 (dd, 1H).

### Step B: 3,4-Bis(4-chlorophenyl)-2-methyl-2-butanol

To a solution of methyl 2,3-bis(4-chlorophenyl)propionate (2.6 g, 8.4 mmol) in ether (20 mL) was added methylmagnesium bromide (3 M in ether, 8.4 mL, 25 mmol) at −10° C., and the reaction was allowed to warm to room temperature over 2 h. The reaction mixture was poured into saturated aqueous ammonium chloride (100 mL), and the product was extracted with EtOAc (3×100 mL). The combined extracts were dried over anhydrous MgSO₄, filtered, and concentrated to dryness to give the title compound, which was used without further purification. ¹H NMR (500 MHz, CD₃OD): δ 7.17 (ABq, 4H), 7.06 (d, 2H), 6.93 (d, 2H), 3.32 (dd, 1H), 2.94 (dd, 1H), 2.84(dd, 1H), 1.20 (s, 3H), 1.16 (s, 3H).

### Step C: N-[2,3-Bis(4-chlorophenyl)-1,1-dimethylpropyl]chloroacetamide

To a solution of 3,4-bis(4-chlorophenyl)-2-methyl-2-butanol (Step B, 1.4 g, 4.5 mmol) and chloroacetonitrile (0.57 mL, 9.1 mmol) in acetic acid (0.7 mL) at −10° C. was added concentrated sulfuric acid (0.31 mL, 14 mmol). After stirring at −10° C. for 15 min and room temperature for 2 h, the reaction mixture was poured onto ice (20 g), and the product was extracted with EtOAc (3×20 mL). The combined extracts were washed with brine/saturated aqueous sodium bicarbonate, dried over anhydrous MgSO₄, filtered, and concentrated to dryness to give the title compound. ¹H NMR (500 MHz, CD₃OD): δ 7.19 (ABq, 4H), 7.06 (d, 2H), 6.95 (d, 2H), 3.93 (ABq, 2H), 3.89 (dd, 1H), 3.10 (dd, 1H), 2.99(dd, 1H), 1.43 (s, 3H), 1.25 (s, 3H). LC-MS: m/e 384 (M+H)⁺(3.9 min).

### Step D: 2-Amino-3,4-bis(4-chlorophenyl)-2-methylbutane hydrochloride

To a solution of N-[2,3-bis(4-chlorophenyl)-1,1-dimethylpropyl]chloroacetamide (Step C, 1.3 g, 3.8 mmol) in ethanol (10 mL) and acetic acid (2 mL) was added thiourea (0.34 g, 4.5 mmol). The reaction was stirred at 80° C. overnight to give a white precipitate. The precipitate was removed by filtration and washed with ethanol (10 mL), and the filtrate was diluted with dilute aqueous sodium hydroxide and extracted with hexane (2×50 mL). The combined extracts were dried over sodium hydroxide, filtered, and concentrated to dryness, and the residue was taken up by hydrogen chloride in dioxane (4 M, 5 mL) and concentrated to dryness to give the title compound. ¹H NMR (500 MHz, CD₃OD): (free amine) δ 7.22-7.14 (m, 4H), 7.06 (d, 2H), 6.96 (d, 2H), 3.22 (dd, 1H), 2.95 (dd, 1H), 2.86(dd, 1H), 1.16 (s, 3H), 1.10 (s, 3H).

### REFERENCE EXAMPLE 26

### 2-Amino-5-methyl-3-phenylhexane hydrochloride salt

### Step A: 4-Methyl-2-phenylpentanoic acid

A solution of 0.25 g (1.84 mmol) of phenylacetic acid in 3.6 mL dry THF was cooled in ice bath and 4 mL 1M lithium bis(trimethylsilyl)amide was added. After 15 min, 0.23 mL (2.02 mmol) of isobutyliodide was added and the cold bath was removed. After stirring the reaction overnight, it was quenched with water and extracted once with EtOAc. The aqueous layer was acidified with 1.2 N HCl and extracted with EtOAc. The EtOAc solution was washed with brine, dried and concentrated to furnish the title compound which was used in the next step without purification. ¹H NMR: (500 MHz, CDCl₃): δ 0.92 (d, 6H), 1.51 (m, 1H), 1.72 (m, 1H), 1.98 (m, 1H), 3.67(m, 1H), 7.0-7.4 (m, 5H).

### Step B: N-Methoxy-N-methyl-4-methyl-2-phenylpentanamide

To a solution of 0.234 g (1.22 mmol) of 4-methyl-2-phenylpentanoic acid in 6 mL CH₂Cl₂ and 2 drops of DMF, 0.12 mL (1.34 mmol) of oxalyl chloride was added. The solution was stirred for 1 h and concentrated. The residue was dissolved in 1 mL CH₂Cl₂ and added to a mixture of 0.142 g N,O-dimethylhydroxylamine hydrochloride in 4 mL CH₂Cl₂and 4 mL saturated NaHCO₃. After stirring for 4 h, the layers were separated and the aqueous layer was extracted with CH₂Cl₂. The combined CH₂Cl₂ layer was washed with brine, dried and concentrated to give the title compound which was used in the next step without purification. ¹H NMR: (500 MHz, CDCl₃): δ 0.94 and 0.96 (2d, 6H), 1.5 (m, 1H), 1.67 (m, 1H), 2.0 (m, 1H), 3.19 (s, 3H), 3.54 (s, 3H), 4.18 (br, 1H), 7.2-7.4 (m, 5H).

### Step C: 5-Methyl-3-phenyl-2-hexanone

To a solution of 75 mg (0.317 mmol) N-methoxy-N-methyl-4-methyl-2-phenylpentanamide in 1 mL dry THF, 0.45 mL 1.4 M methylmagnesium bromide was added. The reaction was stirred for 1 h, quenched with 1.2 N HCl and extracted with EtOAc. The EtOAc solution was washed with brine, dried and concentrated leaving the title compound. ¹H NMR: (500 MHz, CDCl₃): δ 0.95 (2d, 6H), 1.42 (m, 1H), 1.67 (m, 1H), 1.9 (m, 1H), 2.06 (s, 3H), 3.73 (m, 1H), 7.0-7.4 (m, 5H).

### Step D: 5-Methyl-3-phenyl-2-hexanol

A solution of 66 mg (0.345 mmol) of 5-methyl-3-phenyl-2-hexanone in 1 mL MeOH was treated with 16 mg sodium borohydride. After 1.5 h, the reaction was quenched with 1.2 N HCl and concentrated. The residue was partitioned between EtOAc and water. The organic layer was washed with brine, dried and concentrated to yield the crude title compound which was used without purification. ¹H NMR: (500 MHz, CDCl₃): δ 0.88 (2d, 6H), 1.0-1.8 (m, 4H), 1.2 (d, 3H), 2.64 (m, 1H), 3.9 (m, 1H), 7.2-7.4 (m, 5H).

### Step E: 2-Azido-5-methyl-3-phenylhexane

To a solution of 60 mg 5-methyl-3-phenyl-2-hexanol in 2 mL CH₂Cl_{2, 0.163} g (0.62 mmol) of triphenylphosphine and 96 mg (0.31 mmol) of zinc azide pyridine were added. The reaction mixture was cooled in an ice bath and 98 mL (0.62 mmol) of DEAD was added. The cold bath was removed and the solution was stirred for 3 h. The reaction mixture was filtered through a pad of CELITE diatomaceous earth and the pad was rinsed with CH₂Cl₂. The filtrate was concentrated and the residue was purified by prep-TLC using 20% EtOAc-hexane to isolate the title compound. ¹H NMR: (500 MHz, CDCl₃): δ 0.88 (2d, 6H), 1.12 (d, 3H), 1.31 (m, 1H), 1.72 (m, 2H), 2.68 (m, 1H), 3.53 (m, 1H), 7.2-7.4 (m, 5H).

### Step F: 2-Amino-5-methyl-3-phenylhexane

To a solution of 32 mg 2-azido-5-methyl-3-phenylhexane in 1 mL MeOH and 2 drops of 1.2 N HCl, 4 mg PtO₂ was added and the solution was stirred under H2 atmosphere for 2 h. The reaction was filtered through a pad of CELITE diatomaceous earth and the pad was rinsed with MeOH. The combined filtrate was concentrated to give the desired product. ¹H NMR: (500 MHz, CDCl₃): δ 0.86 (m, 6H), 0.99 (d, 3H), 1.25 (m, 1H), 1.54 (m, 1H), 1.77 (m, 1H), 2.73 (m, 1H), 3.19 (m, 1H), 7.2-7.4 (m, 5H).

### REFERENCE EXAMPLE 27

### N-[3-(4-Chlorophenyl)-2-(3,5-difluorophenyl)-1-methylpropyl]amine hydrochloride (Diastereomer α)

The title compounds were prepared following the procedures described for Reference Example 10 substituting methyl phenylacetate with methyl 3,5-difluorophenylacetate (prepared from 3,5-difluorophenylacetic acid and trimethylsilyldiazomethane) at Step A and sodium borohydride in MeOH with lithium tri(sec-butylborohydride in THF at Step E. LC-MS: m/e 296 (M+H)⁺(2.39 min).

### REFERENCE EXAMPLE 28

### N-[2-3-Bromophenyl)-3-(4-chlorophenyl)-1-methylpropyl]amine hydrochloride (Diastereomer α)

The title compounds were prepared following the procedures described for Reference Example 10 substituting methyl phenylacetate with methyl 3-bromophenylacetate (prepared from 3-bromophenylacetic acid and trimethylsilyldiazomethane) at Step A and sodium borohydride in MeOH with lithium tri(sec-butylborohydride in THF at Step E. LC-MS: m/e 338 (M+H)⁺(2.5 min).

### REFERENCE EXAMPLE 29

### N-[2-(3-Chlorophenyl)-3-(4-chlorophenyl)-1-methylpropyl]amine hydrochloride (Diastereomer α)

### Step A: 2-(N-tert-Butoxycarbonyl)amino-4-(4-chlorophenyl)-3-(3-trimethylstannylphenyl)butane

To a solution of 2-(N-tert-butoxycarbonyl)amino-3-(3-bromophenyl)-4-(4-chlorophenyl)butane (intermediate of Reference Example 28, 1.5 g, 3.4 mmol) in 15 mL anhydrous dioxane was added hexamethylditin (1.6 g, 4.8 mmol), triphenylphosphine (18 mg, 0.068 mmol), lithium chloride (0.16 g, 3.8 mmol) and tetrakis(triphenyl-phosphine)palladium (0.20 g, 0.17 mmol). After heating at 95° C. for 7.5 h under nitrogen, the reaction mixture was cooled to room temperature, diluted with EtOAc (100 mL), washed with 10% aqueous potassium fluoride and brine, dried over anhydrous MgSO₄, filtered and concentrated to dryness. The residue was purified by flash column chromatography on silica gel eluted with 20% EtOAc in hexane to afford the title compound. ¹H NMR (500 MHz, CD₃OD): δ 7.3-7.2 (m, 2H), 7.07 (d, J=8.5 Hz, 2H), 7.06-6.99 (m, 2H), 6.86 (d, J=8.5 Hz, 2H), 3.93 (m, 1H), 3.18 (m, 1H), 2.76 (m, 2H), 1.51 (s, 9H), 0.94 (d, J=7.0 Hz, 3H), 0.21 (s, 9H).

### Step B: 2-(N-tert-Butoxycarbonyl)amino-3-(3-chlorophenyl)-4-(4-chlorophenyl)butane

To a solution of 2-(N-tert-butoxycarbonyl)amino-4-(4-chlorophenyl)-3-(3-trimethylstanylphenyl)butane (0.55 g, 1.0 mmol) in 5 mL CH₂Cl₂ at 0° C. was added tert-butoxychloride (freshly prepared, 0.20 mL, 1.1 mmol). The reaction was allowed to warm to room temperature over 2 h, and the resulting mixture was concentrated with 2 g silica gel. The residue was purified by flash column chromatography on silica gel eluted with 10% ether in hexane to afford the title compound. ¹H NMR (500 MHz, CD₃OD): δ 7.25-7.15(m, 2H), 7.11 (d, J=8.5 Hz, 2H), 7.09 (m, 1H), 6.99 (d, J=7.5 Hz, 1H), 6.92 (d, J=8.5 Hz, 2H), 3.88 (m, 1H), 3.19 (dd, J=13.0, 3.5 Hz, 1H), 2.90-2.75 (m, 2H), 1.50 (s, 9H), 0.94 (d, J=6.5 Hz).

### Step C: N-[2-(3-Chloroophenyl)-3-(4-chlorophenyl)-1-methylpropyl]amine hydrochloride (Diastereomer α)

The title compound was prepared following the procedure described for Reference Example 10, Step I. LC-MS: m/e 294 (M+H)⁺(2.82 min).

### REFERENCE EXAMPLE 30

### N-[2-(3-Bromophenyl)-3-(4-chlorophenyl)-1-methylpropyl]amine hydrochloride and N-[3-(4-Chlorophenyl)-2-(3-iodophenyl)-1-methylproryl]amine hydrochloride (1:1 mixture) (Diastereomer α)

### Step A: 2-(N-tert-Butoxycarbonyl)amino-3-(3-bromophenyl)-4-4-chlorophenyl)-butane and 2-(N-tert-Butoxycarbonyl)amino-4-4-chlorophenyl)-3-(3-iodophenyl)butane

To a solution of 2-(N-tert-butoxycarbonyl)amino-3-3-bromophenyl)-4-(4-chlorophenyl)butane (intermediate of Reference Example 28, 2.6 g, 5.9 mmol) in 7 mL anhydrous THF at 0° C. was added methylmagnesium chloride (3 M in THF, 3.9 mL, 12 mmol). After 30 min, the reaction mixture was cooled to −78° C., and was added tert-butyllithium (1.7 M, 10 mL, 17 mmol). After stirring at −78° C. for 2 h, the reaction was allowed to warm to 0° C., and half of the resulting mixture was added to a suspension of iodine (5.0 g, mmol) in 10 mL THF at −40° C. The reaction mixture was allowed to warm to room temperature over 2 h, and was partitioned between ether (100 mL) and saturated aqueous ammonium chloride (100 mL). The organic layer was separated and the aqueous layer extracted with ether (2×50 mL). The combined extracts were washed with dilute aqueous sodium thiosulfate (2×) and brine, dried over anhydrous MgSO₄, filtered and concentrated to dryness. The residue was purified by flash column chromatography on silica gel eluted with 10% EtOAc in hexane to afford the title compounds as a 1:1 mixture.

### Step B: N-[2-(3-Bromophenyl)-3-(4-chlorophenyl)-1-methylpropyl]amine hydrochloride and N-[3-(4-chlorophenyl)-2-(3-iodophenyl)-1-methylpropyl]amine hydrochloride (1:1 mixture) (Diastereomer α)

The title compound was prepared following procedure described for Reference Example 10, Step 1. LC-MS: m/e 338/386/(M+H)⁺(2.6 min).

### REFERENCE EXAMPLE 31

### 2-Amino-4-(4-chlorophenyl)-3-cyclobutylmethoxybutane

### Step A: Methyl 2-diazo-3-(4-chlorophenyl)propanoate

(D,L)-4-Chlorophenylalanine methyl ester (5.0 g, 23.36 mmol) was dissolved in 120 mL chloroform and placed into an oven-dried 3-neck flask equipped with a condenser and an addition funnel. Glacial acetic acid (0.267 mL, 4.672 mmol) was added. Finally, isoamylnitrite (3.8 nL, 28 mmol) was added dropwise while slowly bringing the reaction to reflux (73° C.). The reaction was refluxed for 30 minutes and then cooled to 0° C. The reaction mixture was washed with cold 1 N sulfuric acid solution, cold water, cold saturated aqueous sodium bicarbonate solution, and then cold water again. The organic extracts were dried over MgSO₄, filtered and concentrated under reduced pressure. The crude mixture was purified by flash chromatography (Biotage 40M cartridge, gradient elution using hexane and EtOAc (100:1 to 50:1) to provide a yellow oil, homogeneous by TLC, R_{f}=0.48 (4:1 hexanes:EtOAc). 500 MHz ¹H NMR (CDCl₃): δ 3.65 (s, 2H); 3.83 (s, 3H); 7.22 (d, J=8.5 Hz, 2H), 7.34 (d, J=8.5, 2H).

### Step B: Methyl 3-(4-chlorophenyl)-2-cyclobutylmethoxypropanoate

To a solution of 500 mg (2.23 mmol) of methyl-2-diazo-3-4-chlorophenyl)propanoate (obtained from Step A) and 1.05 mL (5 eq; 11.1 mmol) of cyclobutanemethanol in 5 mL benzene in a pressure tube was added 10 mg (1 mole %) of Rh₂(OAc)₄ catalyst. The tube was sealed and heated to 90° C. for 1.5 h. The solvents were evaporated under reduced pressure and the crude material was taken up in CH₂Cl₂ and purified by flash chromatography via gradient elution using mixtures of hexane and EtOAc (100:1 to 50:1). This provided the title compound as a clear oil. TLC R_{f}=0.53 (4:1 hexanes:EtOAc). 500 MHz ¹H NMR (CDCl₃): δ 1.68 (m, 2H); 1.85 (m, 1H); 1.88 (m, 1H); 2.01 (m, 2H); 2.53 (sep, 1H); 2.98 (m, 2H); 3.24 (dd, 1H); 3.58 (dd, 1H); 3.76 (s, 3H); 3.98 (dd, 1H); 7.20 (d, 2H); 7.28 (d, 2H).

### Step C: 4-(4-Chlorophenyl)-3-cyclobutylmethoxybutan-2-one

At 0° C., under anhydrous conditions, to a stirred suspension of N,O-dimethylhydroxylaminehydrochloride (732 mg, 7.50 mmol) in 60 mL CH₂Cl₂ was added dimethylaluminum chloride (7.5 mL, 1M solution in hexanes). The solution was allowed to warm to room temperature over a period of one hour. At that point a solution of methyl 2-cyclobutylmethoxy-3-(4-chlorophenyl) propanoate (531 mg, 1.88 mmol, obtained from Step B) in CH₂Cl₂ (8 mL) was added dropwise. The reaction was allowed to stir overnight at room temperature when TLC indicated completion of reaction. The reaction was worked up by the addition of pH=8 phospate buffer (25 mL, approx. 3 mL/mmol of Me₂AlCl) and allowed to stir at room temperature for 30 minutes, diluted with chloroform (75 mL), and the phases were separated. The organic layer was washed with water and dried over MgSO₄. The solvents were evaporated under reduced pressure and the crude product was purified by flash chromatography (gradient elution using hexane and EtOAc, 20:1 to 5:1) to give the Weinreb amide as a clear oil). This purified material (424 mg, 1.36 mmol) was dissolved in 10 mL THF, injected into an oven dried flask, and cooled to 0° C. under nitrogen. Methyl magnesium bromide (1.4 mL 3M solution in ether) was added to the solution dropwise. The reaction was allowed to warm to room temperature. After 4 h the TLC indicated a complete reaction. The reaction was quenched with enough 10% citric acid to bring the pH of the solution to approximately 3. The aqueous layer was extract with ether. The combined organics were washed with water and then dried over MgSO₄. The solvents were evaporated under reduced pressure and the crude material was purified by flash chromatography (hexane:EtOAc, 100:1 to 50:1), resulting in 250 mg the title compound as a clear oil. TLC R_{f}=0.55 (4:1 hexanes:EtOAc). 500 MHz ¹H NMR (CDCl₃): δ 1.71 (m, 2H); 1.84 (m, 1H); 1.91 (m, 1H); 2.01 (m, 2H); 2.17 (s, 3H); 2.53 (sep, 1H); 2.90 (m, 2H); 3.28 (dd, 1H); 3.43 (dd, 1H); 3.81 (dd, 1H).

### Step D: 2-Amino-4-(4-chlorophenyl)-3-cyclobutylmethoxybutane

A solution of 3-cyclobutylmethoxy-4-4-chlorophenyl)butan-2-one (247 mg, 0.925 mmol, obtained from Step C) in 0.5 mL CH₂Cl₂ was added to a stirred suspension of NH₄OAc (715 mg, 9.25 mmol) and NaBH₃CN (35 mg, 0.555 mmol) at room temperature and allowed to stir overnight. The reaction was quenched by the addition of 2.2 mL conc. HCl allowed to stir for 30 minutes. The solvents were evaporated under reduced pressure and the residue was partitioned between ether and water. The aqueous layer was washed two more times with ether. The combined organics were dried over Na₂SO₄. The crude product mixture obtained after filtration and removal of volatiles was purified by flash chromatography, eluting using mixtures of mixtures of CH₂Cl₂ and MeOH (100% CH₂Cl₂, to 5% MeOH in CH₂Cl₂) to provide the title compound as a yellow oil, homogeneous by TLC R_{f}=0.12 (5% MeOH in CH₂Cl₂). 500 MHz ¹H NMR (CDCl₃): δ 1.16 (t, 3H); 1.67 (m, 2H); 1.85 (m, 3H); 2.01 (m, 2H); 2.48 (m, 1H); 2.74 (m, 2H); 2.90 (dd, 1H);3.15 (d quint, 2H); 3.37 (m, 2H). 2-Amino-4-(4-chlorophenyl)-3-methoxy-butane, 2-amino-4-(4-chlorophenyl)-3-ethoxy-butane, 2-amino-4-(4-chlorophenyl)-3-n-propyloxy-butane, 2-amino-4-(4-chlorophenyl)-3-n-pentyloxy-butane, and 2-amino-4-(4-chlorophenyl)-3-cyclopentylmethoxy-butane were prepared according to the procedures described in Reference Example 31 substituting an appropriate alcohol for cyclobutylmethanol in Step B.

### REFERENCE EXAMPLE 32

### 2-Amino-4-(4-chlorophenyl)-3-(1-pyrrolidinyl)-butane hydrochloride

### Step A: Ethyl 3-(4-chlorophenyl)-2-pirrolidin-N-yl-propanoate

While stirring rapidly, to a mixture of (D,L)-4-chlorophenylalanine methyl ester hydrochloride (2.5 g, 10 mmole), 40 mL ethanol and sodium carbonate (3.18 g, 30 mmole) was added dropwise a solution of 1,4-dibromobutane (2.16 g, 10 mmol) dissolved in 20 mL ethanol. The mixture was refluxed overnight. The volatiles were removed under reduced pressure, and the residue was partitioned between water and EtOAc. The aqueous layer was re-extracted with EtOAc thrice. The organic layers were combined and washed tieh water and brine and dried over anhydrous MgSO₄. The crude product obtained after filtration and removal of volatiles was purified via flash chromatography using mixtures of CH₂Cl₂ and MeOH to provide the titled compound as an oil, homogeneous by TLC, R_{f}=0.55 in 95:5 CH₂Cl₂: MeOH. LC/MS m/e=282.1 (M+1). 400 MHz ¹H NMR (CDCl₃) δ 1.12(t, J=7.2 Hz, 3H), 1.72 (m, 4H), 2.67 (m, 1H), 2.76(m, 1H), 3.05 (m, 4H), 3.43 (m, 1H), 4.05 (m, 2H), 7.13 (d, J=8.2 Hz, 2H), 7.24 (d, J=8.2 Hz, 2H)

### Step B: 4-(4-Chlorophenyl)-3-(1-pyrrolidinyl)-butan-2-one

The title compound was prepared according to the procedure of Reference Example 10, Step C except that ethyl 3-(4-chlorophenyl)-2-(1-pyrrolidinyl)-propanoate (from Step A) was the ester used (two steps). TLC R_{f}=0.7 (95:5 CH₂Cl₂:MeOH). LC/MS m/e=252 (M+1). 500 MHz ¹H NMR (CDCI₃) δ 1.86(br s, 4H), 2.03 (s, 3H), 2.66 (m, 2H), 2.78 (m, 2H), 2.98 (dd, J=2.9, 10.3 Hz, 1H), 3.08 (m, 1H), 3.43 (m, 1H), 7.12 (d, J=8.3 Hz, 2H), 7.26 (d, J=8.3 Hz, 2H)

### Step C: 4-(4-Chlorophenyl)-3-pyrrolidin-N-yl-butan-2-one oxime

To a solution of 4-(4-chlorophenyl)-3-pyrrolidin-N-yl-butan-2-one (200 mg, 0.79 mmol, from Step B) dissolved in ethanol (2 mL), was added pyridine (63 mg, 0.79 mmol), and hydroxylamine hydrochloride (78 mg, 1.12 mmol). The mixture was refluxed for 24 h when LC/MS indicated disappearance of all starting material. The mixture was cooled to room temperature, concentrated under reduced pressure, treated with 33% aqueous potassium carbonated, and extracted with chloroform 5 times. The organic layers were combined and filtered over glass wool and dried over potassium carbonate. The filtrated obtained after passing through sintered glass was concentrated to give the oxime, homogeneous by TLC, R_{f}=0.3 in 95:5 CH₂Cl₂:MeOH. LC/MS m/e=267 (M+1). 500 MHz ¹H NMR (CDCl₃) δ 1.73(m, 4H), 1.76 (s, 3H), 2.40 (m, 2H), 2.60 (m, 2H), 2.72 (dd, J=2.7, 10.8 Hz, 1H), 2.94 (dd, J=4.3,8.8 Hz, 1H), 3.03 (dd, J=4.4, 13.3 Hz, 1H), 3.8 (s, 1H), 6.96 (d, J=8.3 Hz, 2H), 7.11 (d, J=8.3 Hz, 2H)

### Step D: 2-Amino-4-(4-chlorophenyl)-3-pyrrolidin-N-yl-butane hydrochloride

At room temperature, to a solution of 4-(4-chlorophenyl)-3-pyrrolidin-N-yl-butan-2-one oxime (173 mg, 0.648 mmol, from Step C) in 1.8 mL anhydrous THF was added dropwise a 1M solution of lithium aluminum hydride in THF (0.778 mmole). The mixture was refluxed for 20 h. The reaction was quenched by addition of saturated aqueous sodium sulfate (0.1 mL), and stirred overnight. This mixture was filtered over a pad of CELITE diatomaceous earth, and the filtrate was concentrated to dryness. The mass spectrum of this material looked very messy, so the HCl salt was prepared (by addition of a HCl(g) in ether solution) in attempt to clean up the mess. By NMR, the reductive amination provided a ˜1:1 mixture of the two diastereomeric pairs of amines. This HCl salt was rather sticky and difficult to work with and therefore was used in the ensuing coupling experiment without further purification. LC/MS m/e=253 (M+1). 500 MHz ¹H NMR (CD₃OD) δ 1.56, 1.59 (2 d, J=7.2 Hz, 3H), 2.03 (m, 6H), 2.08 (m, 2H), 3.20-4.00 (m, 3H), 7.43 (m, 4H)

### REFERENCE EXAMPLE 33

### Benzyl 3-amino-2-(4-chlorobenzyl)butyrate

### Step A: Benzyl 2-(4-chlorobenzyl)-3-ketobutyrate

Benzyl acetoacetate (1.92 g, 10 mmole) and 4-chlorobenzylbromide (2.05 g, 10 mmole) were dissolved in 40 mL anhydrous THF and cooled to −10° C. To this mixture was added dropwise slowly a solution of solution of sodium hexamethyl disilazide (0.5M solution in THF). Monoalkylation occurred almost exclusively of bisalkylation between −10 and 5° C. After quenching with water, the organics were extracted with EtOAc three times. The combined organic layer was washed with brine and dried over anhydrous MgSO₄. The crude product obtained after filtration and removal of volatiles was purified via flash chromatography using gradient elution (mixtures of hexane and EtOAc) to provide of the title compound as a clear yellow liquid, homogeneous by TLC, R_{f}=0.4 in 4:1 hexane:EtOAc. By NMR, this compound, this compound exists in a ˜4:1 ratio of the keto:enol forms. 400 MHz ¹H NMR (CDCl₃) δ 2.08, 2.18(2 s, 3H), 3.15 (m, 2H), 3.80 (t, J=7.5 Hz, 0.8 H), 5.14, 5.17 (2 s, 2H), 7.05-7.39 (m, 9H).

### Step B: Benzyl 3-amino-2-(4-chlorobenzyl)butyrate

Benzyl 2-(4-chlorobenzyl)-3-ketobutyrate (317 mg, 1 mmole, obtained from Step A) was added to a cooled mixture of 7M ammonia in MeOH (2.42 mL) and glacial acetic acid (1.6 mL). To this solution, at ˜10° C., was added sodium cyanoborohydride (101 mg, 1.75 mmol) in small portions. This mixture was stirred at room temperature for 40 h. The excess sodium cyanoborohydride was destroyed by the addition of 6M HCl (to pH 1). The residue obtained after removal of volatiles was taken up in a minimal amount of water and extracted with ether. The aqueous layer was basified to pH 10 using solid KOH. This layer was then saturated with sodium chloride and then extracted with EtOAc. Further analyses of the ether and the EtOAc layers suggest that the desired product resides the EtOAc layer. This material was used in the ensuing coupling reaction without further purification. Proton NMR spectrum show that the two pairs of diastereomers are obtained in ˜1:1 ratio, homogeneous by TLC, R_{f}=0.4 in 95:5 CH₂Cl₂:MeOH. LC/MS m/e=318 (M+1). 400 MHz ¹H NMR (CDCl₃) δ 1.27, 1.29 (2 d, J=7 Hz, 3H), 2.85 (m, 1H), 3.03 (m, 1H), 3.15 (m, 1H), 3.55 (m, 1H), 4.85 (br, 2H), 5.00-5.18 (m, 2H), 7.0-7.2 (m, 9H).

### REFERENCE EXAMPLE 34

### 2-Amino-4-(4-chlorophenyl)-3-cyclopentylbutane

### Step A: Methyl 3-(4-chlorophenyl)-2-cyclopentylpropanoate

A mixture of methyl cyclopentylacetate (3.52 g, 25 mmol) and 4-chlorobenzyl bromide (4.75 g, 23 mmol) was dissolved in 100 mL THF in an oven-dried flask. The solution was cooled to −40° C. and 23 mL 1M NaHMDS solution in hexanes was added slowly over an hour while maintaining the temperature at −40° C. The solution was then stirred for an additional 3 h at −40° C. The reaction was quenched at −40° C. with enough 10% citric acid solution to bring the pH to ˜3.5. The aqueous layer was extracted with ether three times. The combined organics were washed with water and dried over MgSO₄. The solvents were evaporated under reduced pressure and the crude material was purified by flash chromatography [Biotage 40 M, gradient elution using mixtures of hexane and EtOAc (from 0-1% EtOAc)]. This provided a light brown oil, which is a 3:1 ratio of the title compound:methyl cyclopentylacetate based on the methyl ester peak integrations. TLC of the desired product: R_{f}=0.34 in 20:1 hexane:EtOAc. The complete separation of the title compound from the starting material was not practical in this case, as they had overlapping R_{f}'s on the TLC. Therefore, this mixture was carried on to the next step.

### Step B: 3-(4-Chlorophenyl)-2-cyclopentylpropanioc acid

The mixture of methyl esters from Step A (3.41 g, 14.48 mmol of methyl 3-(4-chlorophenyl)-2-cyclopentylpropanoate—assuming 3:1 mixture obtained in Step A.) was dissolved in 10 mL DMSO and 4 mL distilled water. Then powdered KOH (3.25 g, 57.92 mmol) was added and the solution was stirred overnight at room temperature. The next day the pH was brought to 2 with 2 N HCl. The aqueous layer was extracted 3 times with ether. The combined organic extracts were dried over anhydrous sodium sulfate. Filtration and evaporation of volatiles provided the mixture of acids as an oil. 500 MHz ¹H NMR (CDCl₃): δ 1.28 (m, 2H), 1.64 (m, 6H), 2.06 (m, 1H), 2.47 (m, 1H), 2.86 (t, 2H).

### Step C: 3-(4-Chlorophenyl)-2-cyclopentyl-N,O-dimethyl-propanamide

The mixture of acids obtained in Step B (3.21 g, 14.48 mmol of the desired acid—based on assumption of 3:1 mixture from Step B) was dissolved in 75 mL CH₂Cl₂. While being stirred rigorously, N,O-dimethylhydroxylamine hydrochloride (1.56 g, 15.95 mmol), 1-ethyl-3-(3-dimethylaminopropyl)carbodiimide (3.06 g, 16.0 mmol), diisopropylethylamine (5.56 mL, 31.90 mmol), and a catalytic amount of 4-(dimethylaminopyridine) were added sequentially. Stirring was continued overnight at room temperature. The next day the reaction mixture was diluted with EtOAc, treated with water, and the phases were separated. The aqueous layer was re-extracted with EtOAc twice. The combined organic layers were washed with water three times and then with saturated brine. The organic layer was dried over MgSO₄, filtered, and the solvents were removed under reduced pressure. The crude material was purified by flash chromatography [Biotage 40 M column, gradient elution using mixtures or hexanes and EtOAc (100:1 to 20:1] to provide the title compound cleanly as an oil. TLC R_{f}=0.31 (4:1 hexanes:EtOAc). LC/MS m/e 295.9 (M+1). 500 MHz ¹H NMR (CDCl₃): δ 1.27(m, 2H), 1.64 (m, 6H), 1.97 (m, 1H), 2.13 (q, 1H), 2.81 (d, 1H), 2.97 (d, 1H), 3.07 (s, 3H), 3.17 (s, 3H). LC/MS m/e 295.9 (M+1).

### Step D: 4-(4-Chlorophenyl)-3-cyclopentylbutan-2-one

3-(4-Chlorophenyl)-2-cyclopentyl-N,O-dimethyl-propanamide (514 mg, 1.737 mmol, obtained from Step C) was dissolved in 15 mL anhydrous THF and injected into an oven dried flask under nitrogen. The solution was cooled to 0° C. and CH₃MgBr (1 M in ether) was added dropwise. The ice bath was removed and the reaction was allowed to warm to room temperature and stirred for a total of 4 h. TLC indicated a nearly complete reaction. The reaction was quenched with enough 10% citric acid to bring the pH of the solution to 3. The aqueous layer was extracted 3 times with ether and the extracts were dried over anhydrous MgSO₄. The solution was filtered and the solvents were removed under reduced pressure. The crude material was purified by flash chromatography (30 mL silica; 100:1 to 50:1 hexanes: EtOAc) to provide 351 mg the title compound as an oil. TLC R_{f}=0.49 (4:1 hexanes: EtOAc). 500 MHz ¹H NMR (CDCl₃): δ 1.23 (m, 3H), 1.58 (m, 1H), 1.71 (m, 3H), 1.91 (s, 3H), 1.93 (m, 1H), 2.05 (m, 1H), 2.68 (m, 1H), 2.84 (m, 2H).

### Step E: 2-Amino-4-(4-chlorophenyl)-3-cyclopentylbutane

The title compound was prepared according to the procedure of Reference Example 10, Step D, except that 4-(4-chlorophenyl)-3-cyclopentylbutan-2-one (obtained form Step D) was used as the starting material. LC/MS m/e 251.9 (M+1); 500 MHz ¹H NMR (CDCl₃): δ 0.93 (m, 1H), 1.29 (q, 3H), 1.29 (m, 2H), 1.61 (m, 4H), 1.87 (m, 3H), 2.62 (m, 1H), 2.80 (m, 1H), 3.26 and 3.48 (m, 1H).

2-Amino-4-(4-chlorophenyl)-3-ethyl-butane and 2-amino-4-(4-chlorophenyl)-3-isopropyl-butane were also prepared according to the procedures described in Reference Example 34 substituting the appropriate ester for methyl cyclopentylacetate in Step A.

### REFERENCE EXAMPLE 35

### 2-Amino-3-(1-(1,2,3-triazolyl))-4-(4-chlorophenyl)butane

### Step A: Benzyl 2-(1-(1,2,3-triazolyl))acetate

A mixture of 1,2,3-triazole (2.07 g, 30 mmol), benzyl bromoacetate (6.9 g, 30 mmol), and diisopropylethylamine (5,1 mL, 30 mmol) in 40 mL CH₂Cl₂ was stirred overnight at room temperature. This mixture was then diluted with ether until no further precipitate formed. The solid was filtered and washed with ether. The filtrate was concentrated and the residue was purified on silica gel using 10% hexane in CH₂Cl₂ to give the title compound's isomer, benzyl 2-(2-(1,2,3-triazolyl)acetate as amorphous solid. Further elution with a solvent mixture containing equal amounts of ether and CH₂Cl₂ gave the title compound as amorphous solid. ¹H NMR (400 MHz, CDCl₃):δ 2.251(s, 2H0, 7.267-7.390(m, 5H), 7.723(s, 1H), 7.785(s,1H).

### Step B: 2-(1-(1,2,3-triazolyl))acetic acid

Palladium hydroxide (20% on carbon, 800 mg) was added to a solution of benzyl 2-(1-(1,2,3-triazolyl))acetate (Step A, 8.68 g, 39.9 mmnol) in 150 mL MeOH and the mixture was hydrogenated overnight on a Parr shaker under an atmosphere of hydrogen at room temperature and 45 psi. The catalyst was filtered through a bed of CELITE diatomaceous earth and washed with MeOH. The filtrate was concentrated to give a solid, which was dried in vacuo at 50° C. for 36 h resulting in the title compound. ¹H NMR (400 MHz, CD₃OD):δ 5.3 (s, 2H), 7,75 (s, 1H0, 8.016 (s, 1H).

### Step C: N-Methoxy-N-methyl-2-(1-(1,2,3-triazolyl))acetamide

Oxalyl chloride (0.95 mL, 11 mmol) was added dropwise to a suspension of 2-(1-1,2,3-triazolyl))acetic acid (Step B, 1.27 g, 10 mmol) in 10 mL CH₂Cl₂ containing 0.05 mL DMF. Vigorous effervescence was observed. This mixture was stirred at room temperature for 4 h and cooled to −78° C. A solution of N,O-dimethylhydroxylamine hydrochloride (1.2 g, 13 mmol) and diisopropylethyl amine (6.0 mL, 35 mmol) in 10 mL CH₂Cl₂ was added slowly over 3 min. The mixture was then allowed to warm to room temperature and stirred overnight. The reaction mixture was then diluted with ether until no additional precipitate appeared. The solid was filtered and washed with ether. The filtrate was concentrated and the residue was purified on silica gel using EtOAc as solvent to provide the title compound as amorphous solid. ¹H NMR (400 MHz, CDCl₃):δ 3.252 (s, 3H0, 3.812 (s, 3H), 5.379 (s, 2H), 7.753 & 7.761 (s′s, 2H).

### Step D: N-Methoxy-N-methyl-3-(4-chlorophenyl)-2-(1-(1,2,3-triazolyl))propionamide

Lithium hexamethyldisilazide (1 molar in THF, 8.4 mL, 8.4 mmol) was added dropwise to a solution of N-methoxy-N-methyl-2-(1-(1,2,3-triazolyl))acetamide (Step C, 1.19 g, 7 mmol) in 15 mL THF at −78° C. After additional 30 min stirring, a solution of 4-chlorobenzyl bromide (1.65 g, 8 mmol) in 5 mL THF was added dropwise. The mixture was allowed to warm to room temperature and stirred 5.5 h. This mixture was purified on silica gel using 40% EtOAc in hexane to give the title compound. ¹H NMR (400 MHz, CDCl₃): δ 3.186 (s, 3H), 3.234-3,267 (m, 1H), 3,453-3.506 (m, 1H), 3.582 (s, 3H), 6.145-6.188 (m, 1H), 7.048-7.279 (m, 4H), 7.726 (s, 1H), 7.954 (s, 1H).

### Step E: 2-Azido-3-(1-(1,2,3-triazolyl))-4-(4-chlorophenyl)butane

The product of Step D, N-methoxy-N-methyl-3-(4-chlorophenyl)-2-(1-(1,2,3-triazolyl)propionamide was converted to the title compound following the procedures described in Reference Example 10, Step D-E and Reference Example 12, Step D. ¹H NMR (400 MHz, CDCl₃): δ 1.219-1.246 (d′s 3H), 3.253-4.754 (m, 4H0, 6.866-7.299 (d′s, 4H), 7.313, 7.618, 7.63, & 7.706 (s′s, 2H).

### Step F: 2-Amino-3-(1-(1,2,3-triazolyl))-4-(4-chlorophenyl)butane

Platinum oxide (14 mg) was added to a solution of 2-azido-3-(1-(1,2,3-triazolyl))-4-4-chlorophenyl)butane (Step E, 138 mg, 0.5 mmol) in 4 mL MeOH. This mixture was hydrogenated in an atmosphere of hydrogen using a hydrogen filled balloon for 3 h at room temperature. The catalyst was filtered through a bed of CELITE diatomaceous earth and washed with MeOH. The filtrate was concentrated to give the title compound as oil. ¹H NMR (400 MHz, CDCl₃):δ 1.085-1.174 (d′s 3H), 3.220-3.361 (m, 2H), 3.517-3.563 (m, 1H), 4.379-4.431 (m, 1H), 6.679-7.179 (d′s, 4H), 7.297, 7.40, 7.592 & 7.607 (s′s, 2H).

### REFERENCE EXAMPLE 36

### 2-Amino-3-(1-(1,2,4-triazolyl)-4-4-chlorophenyl)butane

The title compound was prepared according to the procedures described in Reference Example 35 substituting 1,2,4-triazole for 1,2,3-triazole in Step A. The azide was separated by column chromatography on silica gel eluted with 20% hexane in EtOAc.

### REFERENCE EXAMPLE 37

### N-[3-(4-Chlorophenyl)-2-3-methylphenyl)-1-methylpropyl]amine hydrochloride (Diastereomer α)

### Step A: 2-(N-tert-Butoxycarbonyl)amino-4-(4-chlorophenyl)-3-(3-methylphenyl)butane

A mixture of 2-(N-tert-butoxycarbonyl)amino-3-3-bromophenyl)-4-(4-chlorophenyl)butane (intermediate of Reference Example 28, 0.50 g, 1.1 mnmol), tetramethyltin (0.41 g, 2.3 mmol), triphenylphosphine (0.12 g, 0.46 mmol), lithium chloride (0.38 g, 9.1 mmol) and dichlorobis(triphenylphosphine)palladium (0.12 g, 0.17 mmol) in 20 mL anhydrous DMF was heated at 100° C. under nitrogen for 18 h. The reaction mixture was cooled to room temperature, and was partitioned between water (100 mL) and ether (100 mL). The organic layer was separated and the aqueous layer was extracted with ether (100 mL). The combined extracts were dried over anhydrous MgSO₄, filtered and concentrated to dryness, and the residue was purified by flash column chromatography on silica gel eluted with 10% EtOAc in hexane to afford the title compound. ¹H NMR (400 MHz, CD₃OD): δ 7.2-6.8 (m, 8H), 3.84 (m, 1H), 3.16 (m, 1H), 2.80-2.68 (m, 2H), 2.24 (s, 3H), 1.45 (s, 9H), 0.86 (d, 3H). LC-MS: m/e 396 (M+Na)⁺(4.4 min).

### Step B: N-[3-(4-Chlorophenyl)-2-(3-methylphenyl)-1-methylpropyl]amine hydrochloride (Diastereomer α)

The title compound was prepared following the procedure described for Reference Example 10, Step I.

LC-MS: m/e 274 (M+H)⁺(2.5 min).

### REFERENCE EXAMPLE 38

### N-[3-(4-Chlorophenyl)-2-(3-trifluoromethylphenyl)-1-methylpropyl]amine hydrochloride (Diastereomer α)

The title compound was prepared following the procedure described in Reference Example 12 substituting fluorophenylacetic acid with 3-trifluoromethylphenylacetic acid at Step A. LC-MS: m/e 328 (M+H)⁺(2.6 min).

### REFERENCE EXAMPLE 39

### N-[3-(5-Chloro-2-pyridyl)-2(S)-phenyl-1(S)-methylpropyl]amine hydrochloride (Diastereomer α)

### Step A: 5-Chloro-2-methylpyridine

A mixture of 2,5-dichloropyridine (15 g, 0.10 mol), tetramethyltin (15 mL, 0.11 mol), and dichlorobis(triphenylphosphine)palladium (2.0 g, 2.8 mmol) in 200 mL anhydrous DMF was heated at 110° C. under nitrogen for 72 h. The reaction mixture was cooled to room temperature, and was poured into a saturated solution of potassium fluoride (200 mL). The resulting mixture was partitioned between water (500 mL) and ether (500 mL). The organic layer was separated and the aqueous layer was extracted with ether (200 mL). The combined extracts were dried over anhydrous MgSO₄, filtered and concentrated to dryness, and the residue was purified by flash column chromatography on silica gel eluted with 2 to 10% ether in hexane to afford the title compound. ¹H NMR (500 MHz, CD₃OD): δ 8.41 (d, 1H), 7.75 (dd, 1H), 7.30 (d, 1H), 2.53 (s, 3H).

### Step B: 4-(5-Chloro-2-pyridyl)-3(S)-phenyl-2(R)-butanol

To a solution of 5-chloro-2-methylpyridine (Step A, 1.1 g, 8.7 mmol) in 15 mL anhydrous ether was added phenyl lithium (1.8 M in cyclohexane/ether, 7.2 mL, 13 mmol) at 0° C., and the reaction was stirred at room temperature for 30 min. The resulting mixture was cooled back to 0° C., and was added (1R,2R)-1-phenylpropylene oxide (2.3 g, 17 mmol), and the reaction was allowed to warm to room temperature overnight. The reaction mixture was partitioned between EtOAc (100 mL) and water (100 mL). The organic layer was separated and the aqueous layer extracted with EtOAc (2×100 mL). The combined organic extracts were dried over anhydrous MgSO₄, filtered, and concentrated to dryness, and the residue was purified by flash column chromatography on silica gel eluted with 10 to 40% EtOAc in hexane to afford the title compound. ¹H NMR (500 MHz, CD₃OD): δ 8.28 (d, 1H), 7.59 (dd, 1H), 7.25-7.12 (m, 5H), 7.05 (d, 1H), 4.03 (m, 1H), 3.29 (dd, 1H), 3.19 (dd, 1H), 3.12 (m, 1H), 1.12 (d, 3H).

### Step C: 2(S)-Azido-4-(5-chloro-2-pyridyl)-3(S)-phenylbutane

To a mixture of 4-(5-chloro-2-pyridyl)-3-phenyl-2-butanol (Step B, 0.24 g, 0.92 mmol), triphenylphosphine (1.5 g, 1.4 mmol) and diphenylphosphoryl azide (0.30 mL, 1.4 mmol) in 5 mL anhydrous THF was added diethylazodicarboxylate (0.24 mL, 1.4 mmol). After stirring at room temperature overnight, the resulting mixture was concentrated with silica gel (10 g) and the residue was loaded onto a silica gel column. Elution with 5 to 15% EtOAc in hexane afforded the title compound. ¹H NMR (500 MHz, CD₃OD): δ 8.35 (d, 1H), 7.52 (dd, 1H), 7.25-7.05 (m, 5H), 6.95 (d, 1H), 3.81 (m, 1H), 3.48 (m, 1H), 3.15-3.05 (m, 2H), 1.14 (d, 3H).

### Step D: N-[3-(5-Chloro-2-pyridyl)-2(S)-phenyl-1(S)-methylpropyl]amine, hydrochloride

The product of Step C (0.20 g, 0.70 mmol) was converted to the title compound following the procedure described in Reference Example 10, Steps H-I, except hydrogen chloride in dioxane (4 M) was used in place of hydrogen chloride in EtOAc. ¹H NMR (500 MHz, CD₃OD): δ 8.75 (d, 1H), 8.19 (dd, 1H), 7.55 (d, 1H), 7.4-7.2 (m, 5H), 3.78 (m, 1H), 3.62 (dd, 1H), 3.48 (m, 1H), 3.43 (dd, 1H), 1.22 (d, 3H). LC-MS: m/e 261 (M+H)⁺(2.2 min).

### REFERENCE EXAMPLE 40

### N-[2-(3-Bromophenyl)-3-(5-chloro-2-pyridyl)-1-methylpropyl]amine hydrochloride (Diastereomer α)

### Step A: 3-Bromophenylacetone

To a solution of N-methoxy-N-methylacetamide (10 g, 100 mmol) in 100 mL anhydrous ether at 0° C. was added 3-bromobenzylrnagnesium bromide (0.25 M in ether, 200 mL, 50 mmol). The reaction was allowed to warm to room temperature overnight and was quenched by the addition of saturated ammonium chloride (100 mL). The organic layer was separated and the aqueous layer was extracted with hexane (100 mL). The combined extracts were dried over anhydrous MgSO₄, filtered and concentrated to dryness to afford the title compound. ¹H NMR (500 MHz, CD₃OD): δ 7.45-7.40 (m, 2H), 7.26 (t, 1H), 7.19 (d, 1H), 2.20 (s, 3H).

### Step B: 3-(3-Bromophenyl)-4-(5-chloro-2-pyridyl)-2-butanone

A suspension of 5-chloro-2-methylpyridine (Reference Example 18, Step A, 6.4 g, 50 mmol) and N-bromosuccinimide (12.5 g, 70 mmol) in 100 mL carbon tetrachloride was heated to gentle reflux (bath temperature 90° C.), and 2,2′-azobisisobutyronitrile (0.74 g) was added in several portions over 30 min. After stirring at this temperature for 5 h, the reaction mixture was concentrated. The resulting slurry was diluted with EtOAc (100 mL) and was washed with water (100 mL), saturated aqueous sodium bicarbonate/saturated aqueous sodium thiosulfate, and brine. The organic solution was dried over anhydrous sodium sulfate, filtered, and concentrated to dryness, and the residue was purified by flash column chromatography on silica gel eluted with 2 to 15% ether in CH₂Cl₂/hexane (1:1) to afford 2-bromomethyl-5-chloropyridine (6.0 g, 60%), which was used immediately for the ensuing reaction. Thus, to a vigorously stirred solution of 2-bromomethyl-5-chloropyridine (6.0 g, 29 mmol) and 3-bromophenyl acetone (Step A, 6.0 g, 28 mmol) and tetrabutylammonium iodide (20 mg) in 30 mL CH₂Cl₂ at −78° C. was added cesium hydroxide monohydrate (10 g, 60 mmol), and the reaction was allowed to slowly warm to room temperate overnight. The reaction mixture was partitioned between EtOAc (100 mL) and water (100 mL). The organic layer was separated and the aqueous layer extracted with EtOAc (2×100 mL). The combined organic extracts were dried over anhydrous sodium sulfate, filtered, and concentrated to dryness, and the residue was purified by flash column chromatography on silica gel eluted with 5 to 40% EtOAc in hexane to afford the title compound. ¹H NMR (500 MHz, CD₃OD): δ 8.44 (d, 1H), 7.66 (dd, 1H), 7.46-7.41 (m, 2H), 7.24 (t, 1H), 7.22 (d, 1H), 7.15 (d, 1h), 4.42 (dd, 1H), 3.54 (dd, 1H), 3.07 (dd, 1H), 2.12 (s, 3H). LC-MS: m/e 338 (M+H)⁺(3.0 min).

### Step C: 3-(3-Bromophenyl)-4-(5-chloro-2-pyridyl)-2-butanol

To a solution of 3-(3-bromophenyl)-4-(5-chloro-2-pyridyl)-2-butanone (Step B, 6.7 g, 20 mmol) in 50 mL anhydrous THF at −78° C. was added lithium tri(sec-butyl)borohydride (1.0 M in THF, 30 mL, 30 mmol), and the reaction was allowed to warm to room temperature overnight. The reaction was cooled to 0° C., and was carefully added 2 M hydrochloric acid (50 mL), and the resulting mixture was partitioned between hexane (200 nL) and water (200 mL). The aqueous layer was separated and the organic layer extracted with 2 M hydrochloric acid (2×100 mL). The combined aqueous extracts were neutralized with 5 N aqueous sodium hydroxide (pH>12), and was extracted with EtOAc (2×200 mL). The combined extracts were dried over anhydrous sodium sulfate, filtered, and concentrated to dryness to afford the title compound.

### Step D: N-[2-(3-Bromophenyl)-3-(5-chloro-2-pyridyl)-1-methylpropyl]amine, hydrochloride

The product of Step C (5.9 g, 17 mmol) was converted to the title compound following the procedure described in Reference Example 39, Steps C-D. LC-MS: m/e 338 (M+H)⁺(2.3 min).

### REFERENCE EXAMPLE 41

### N-[3-(5-Chloro-2-pyridyl)-2-(3-chlorophenyl)-1-methylpropyl]amine hydrochloride (Diastereomer α)

The title compound was prepared following the procedure described in Reference Example 28 substituting 2-(N-tert-butoxycarbonyl)amino-3-bromophenyl-4-(4-chlorophenyl)butane with 2-N-tert-butoxycarbonyl)amino-3-bromophenyl-4-(5-chloro-2-pyridyl)butane (intermediate of Reference Example 40, Step D) at Step A. LC-MS: m/e 295 (M+H)⁺(2.0 min).

### REFERENCE EXAMPLE 42

### N-[2-(5-Bromo-2-pyridyl)-3-(4-chlorophenyl)-1-methylpropyl]amine hydrochloride (Diastereomer α)

### Step A: 5-Bromo-3-pyridylacetone

A mixture of 3,5-dibromopyridine (50 g, 0.21 mol), isopropenyl acetate (26 mL, 0.23 mmol), tris(dibenzylideneacetone)dipalladium (1.0 g, 1.1 mmol) and 2-(diphenylphosphino)-2′(N,N-dimethylamino)biphenyl (1.6 g, 4.2 mmol) in 400 mL toluene was heated at 100° C. under nitrogen for 2 h. The reaction mixture was cooled to room temperature, and was concentrated to about 100 mL. The resulting mixture was loaded onto a silica gel column, which was eluted with 0 to 60% EtOAc in hexane to afford the title compound. ¹H NMR (500 MHz, CD₃OD): δ 8.54 (br s, 1H), 8.33 (br s, 1H), 7.88 (br s, 1H), 3.90 (s, 2H), 2.25 (s, 3H).

### Step B: 3-(5-Bromo-3-pyridyl)-4-(4-chlorophenyl)-2-butanol

The title compound was prepared following the procedures described in Reference Example 40, Step B-C, substituting 2-bromomethyl-5-chloropyridine with 4-chlorobenzyl chloride and 3-bromophenylaceatone with 5-bromo-3-pyridylacetone (Step A). ¹H NMR (500 MHz, CD₃OD): δ 8.43 (d, 1H), 8.24 (d, 1H), 7.98 (dd, 1H), 7.17 (d, 2H), 7.07 (d, 2H), 4.04 (m, 1H), 3.16 (dd, 1H), 3.0-2.9 (m, 2H), 1.04 (d, 3H).

### Step C: N-[2-(5-Bromo-3-pyridyl)-3-(4-chlorophenyl)-1-methylpropyl]amine hydrochloride (Diastereomer α)

The title compound was prepared following the procedure described for Reference Example 11, Step B.

LC-MS: m/e 339 (M+H)⁺(2.5 min).

### REFERENCE EXAMPLE 43

### N-[2-(5-Bromo-3pyridyl)-3-(4-fluorophenyl)-1-methylpropyl]amine hydrochloride (Diastereomer α)

The title compound was prepared following the procedure described for Reference Example 42 substituting 4-chlorobenzyl chloride with 4-fluorobenzyl chloride at Step B. LC-MS: m/e 323 (M+H)⁺(2.3 min).

### REFERENCE EXAMPLE 44

### N-[3-(4-Chlorophenyl)-2-(5-cyano-3-pyridyl)-1-methylpropyl]amine hydrochloride (Diastereomer α)

### Step A: 5-Cyano-3-pyridylacetone

The title compound was prepared following the procedure described for Reference Example 42 substituting 3,5-dibromopyridine with 5-bromonicotinonitrile (5-bromo-3-cyanopyridine) at Step A. ¹H NMR (400 MHz, CD₃OD): δ 8.89 (d, 1H), 8.60 (d, 1H), 8.02 (t, 1H), 3.98 (s, 2H), 2.24 (s, 3H).

### Step B: N-[3-(4-Chlorophenyl)-2-(5-cyano-2-pyridyl)-1-methyllropyl]amine hydrochloride (Diastereomer α/β 5:1)

The title compound was prepared following the procedure described for Reference Example 19 substituting 3-pyridylacetone with 5-cyano-3-pyridylacetone (Step A). LC-MS: m/e 286 (M+H)⁺(1.9 min).

### REFERENCE EXAMPLE 45

### N-[2-(5-Cyano-3-pyridyl)-3-(4-fluorophenyl)-1-methylpropyl]amine hydrochloride (Diastereomer α)

The title compound was prepared following the procedure described for Reference Example 44 substituting 4-chlorobenzyl chloride with 4-fluorobenzyl chloride at Step B. LC-MS: m/e 270 (M+H)⁺(2.2 min).

### REFERENCE EXAMPLE 46

### N-[2-(5-Cyano-3-pyridyl)-3-(3,4-difluorophenyl)-1-methylpropyl]amine hydrochloride (Diastereomer α)

The title compound was prepared following the procedure described for Reference Example 44 substituting 4-fluorobenzyl chloride with 3,4-difluorobenzyl chloride at Step B. LC-MS: m/e 288 (M+H)⁺(2.3 min).

### REFERENCE EXAMPLE 47

### N-[3-(3-Chloronhenyl)-2-(5-cyano-3-pyridyl)-1-methylpropyl]amine hydrochloride (Diastereomer α)

The title compound was prepared following the procedure described for Reference Example 44 substituting 4-fluorobenzyl chloride with 3-chlorobenzyl chloride at Step B. LC-MS: m/e 286 (M+H)⁺(2.4 min).

### REFERENCE EXAMPLE 48

### N-[3-(4-Chlorophenyl)-2-(5-chloro-3-pyridyl)-1-methylpropyl]amine hydrochloride (Diastereomer α)

### Step A: 5-Chloro-3-pyridylacetone

The title compound was prepared following the procedure described for Reference Example 42 substituting 3,5-dibromopyridine with 3,5-dichloropyrdine and 2-(diphenylphosphino)-2′(N,N-dimethylamino)biphenyl with 2-di-t-butylphosphino)biphenyl at Step A. ¹H NMR (500 MHz, CD₃OD): δ 8.42 (d, 1H), 8.27 (d, 1H), 7.73 (dd, 1H), 3.90 (s, 2H), 2.25 (s, 3H).

### Step B: N-[3-(4-Chlorophenyl)-2-(5-chloro-3-pyridyl)-1-methylpropyl]amine hydrochloride (Diastereomer α)

The title compound was prepared following the procedure described for Reference Example 42, Step B-C substituting 5-bromo-3-pyridylacetone with 5-chloro-3-pyridylacetone at Step B. LC-MS: m/e 295 (M+H)⁺(1.9 min).

### REFERENCE EXAMPLE 49

### N-[2-(5-Chloro-3-pyridyl)-3-(4-fluorophenyl)-1-methylpropyl]amine hydrochloride (Diastereomer α)

The title compound was prepared following the procedure described for Reference Example 48 substituting 4-chlorobenzyl chloride with 4-fluorobenzyl chloride at Step B. LC-MS: m/e 279 (M+H)⁺(2.3 min).

### REFERENCE EXAMPLE 50

### 2-Amino-3-(5-chloro-3-pyridyl)-5-methylhane, Hydrochloride Salt (Diastereomer α/β 6:1)

The title compound was prepared following the procedure described for Reference Example 48 substituting 4-chlorobenzyl chloride with 1-iodo-2-methylpropane at Step B. LC-MS: m/e 227 (M+H)⁺(2.2 min).

### REFERENCE EXAMPLE 51

### N-[2-(5-Chloro-3-pyridyl)-3-cyclobutyl-1-methylpropyl]amine hydrochloride (Diastereomer α/β 6:1)

The title compound was prepared following the procedure described for Reference Example 48 substituting 4-chlorobenzyl chloride with (bromomethyl)cyclobutane at Step B. LC-MS: m/e 239 (M+H)⁺(2.3 min).

### REFERENCE EXAMPLE 52

### N-[3-(4-Chlorophenyl)-2-(3-cyanophenyl)-1-methylpropyl]amine hydrochloride (Diastereomer α)

### Step A: 3-Cyanophenylacetone

The title compound was prepared following the procedure described for Reference Example 28 substituting 3,5-dibromopyridine with 3-bromobenzonitrile and 2-diphenylphosphino)-2′-(N,N-dimethylamino)biphenyl with 2-(dicyclohexylphosphino)-2′-(N,N-dimethylamino)biphenyl at Step A. ¹H NMR (500 MHz, CD₃OD): δ 7.6 (m, 1H), 7.56 (br s, 1H), 7.50-7.48 (m, 2H), 3.88 (s, 2H), 2.21 (s, 3H).

### Step B: N-[3-(4-Chlorophenyl)-2-(3-cyanophenyl)-1-methylpropyl]amine hydrochloride (Diastereomer α)

The title compound was prepared following the procedures described for Reference Example 42 substituting 5-bromo-3-pyridylacetone with 3-cyanophenylacetone at Step B. LC-MS: m/e 285 (M+H)⁺(2.2 min).

### REFERENCE EXAMPLE 53

### N-[3-(4-Chlorophenyl)-2-(5-fluoro-3-pyridyl)-1-methylpropyl]amine hydrochloride (Diastereomer α)

### Step A: 5-fluoro-3-pyridylacetone

The title compound was prepared following the procedure described for Reference Example 42 substituting 3,5-dibromopyridine with 3-fluoro-5-trifluoromethanesulfonyloxypyridine (prepared form 3-fluoro-5-hydroxypyrdine and triflic anhydride) and 2-(diphenylphosphino)-2′(N,N-dimethylamino)biphenyl with 2-(dicyclohexylphosphino)-2′(N,N-dimethylamino)biphenyl at Step A. ¹H NMR (500 MHz, CD₃OD): δ 8.34 (d, 1H), 8.22 (br s, 1H), 7.50 (ddd, 1H), 3.93 (s, 2H), 2.25 (s, 3H).

### Step B: N-[3-(4-Chlorophenyl)-2-(5-chloro-3-pyridyl)-1-methylpropyl]amine hydrochloride (Diastereomer α)

The title compound was prepared following the procedure described for Reference Example 42, Step B-C substituting 5-bromo-3-pyridylacetone with 5-fluoro-3-pyridylacetone at Step B. LC-MS: m/e 279 (M+H)⁺(2.4 min).

### REFERENCE EXAMPLE 54

### N-[3-(4-Chlorophenyl)-2-(5-methyl-3-pyridyl)-1-methylpropyl]amine hydrochloride (Diastereomer α)

The title compound was prepared following the procedure described for Reference Example 28 substituting 2-(N-tert-butoxycarbonyl)arnino-3-3-bromophenyl)-4-(4-chlorophenyl)butane with 2-(N-tert-butoxycarbonyl)amino-3-(5-bromo-3-pyridyl)-4-(4-chlorophenyl)butane (intermediate of Reference Example 42, Step B) at Step A. LC-MS: m/e 275 (M+H)⁺(1.3 min).

### REFERENCE EXAMPLE 55

### N-[2-(3-Bromo-5-fluorophenyl)-3-(4-Chlorophenyl)-1-methylpropyl]amine hydrochloride (Diastereomer α)

### Step A: 3-Bromo-5-fluorophenylacetone

The title compound was prepared following the procedure described for Reference example 42 substituting 3,5-dibromopyridine with 1,3-dibromo-5-fluorobenzene and 2-diphenylphosphino)-2′-(N,N-dimethylamino)biphenyl with 1,140 -bis(diphenylphosphino)ferrocene at Step A. ¹H NMR (500 MHz, CD₃OD): δ 7.23 (d, 1H), 7.22 (s, 1H), 6.96 (d, 1H), 3.81 (s, 2H), 2.20 (s, 3H).

### Step B: N-[2-(3-Bromo-5-fluorophenyl)-3-(4-chlorophenyl)-1-methylpropyl]amine hydrochloride (Diastereomer α)

The title compound was prepared following the procedure described for Reference Example 42, Steps B-C substituting 5-bromo-3-pyridylacetone with 3-bromo-5-fluorophenylacetone (Step A). LC-MS: m/e 356 (M+H)⁺(2.9 min).

### REFERENCE EXAMPLE 56

### N-[2-3-Bromo-5-fluorophenyl)-3-(4-fluorophenyl)-1-methylpropyl]amine hydrochloride (Diastereomer α)

The title compound was prepared following the procedures described for Reference Example 55 substituting 4-chlorobenzyl chloride with 4-fluorobenzyl chloride at Step B. LC-MS: m/e 340 (M+H)⁺(2.8 min).

### REFERENCE EXAMPLE 57

### 2-Amino-3-indolin-N-yl-4(4-chloro)phenylbutane

### Step A: Ethyl 3-(4-chlorophenyl)-2-indolin-N-ylpropanoate

In an oven-dried flask under an atmosphere of nitrogen, 1.1 g LiOH.H₂O (26.25 mmol) in DMF (20 mL) was added to a stirring suspension of 4 angstrom molecular sieves. After 30 minutes of stirring at room temperature 2.8 mL (25 mmol) indoline was added dropwise. After one hour at room temperature 2.9 mL (26.25 mmol) Ethyl bromoacetate was added dropwise. After 1.5 h the solid material was filtered and the residue was washed with copious amounts of EtOAc. The organics were washed 3 times with water and the organic material was dried over MgSO₄. The solvents were evaporated under reduced pressure. The crude rnaterial was then dissolved in 75 mL anhydrous THF, charged into an oven dried round bottom under an atmosphere of nitrogen, cooled to −78° C., and then treated with 26.25 mL a 1M solution of NaHMDS. The solution was allowed to stir for 30 minutes at −78° C. after which the enolate was quenched with 5.4 g (26.25 mmol) of parachlorobenzyl bromide (solution in 25 mL anhydrous THF). The reaction was allowed to warm to room temperature overnight. The next day the reaction was quenched with water. The aqueous layer was extracted with 3 large portions of EtOAc. The combined organics were dried over MgSO₄. The solvents were removed under reduced pressure and the residue was purified by flash chromatography which yielded the title compound as a yellow oil. LC/MS m/e=331 (M+1). TLC R_{f}=0.22 (20:1 hexanes:EtOAc). ¹H NMR (500 MHz, CDCl₃): δ 1.11 (t, J=3.55 Hz, 3H), 2.96 (m, 2H), 3.06 (m, 1H), 3.25 (m, 1H), 3.60 (t, 2H), 4.07 (m, 2H), 4.36 (t, J=3.75 Hz, 1H).

### Step B: N,O-dimethyl-3-(4-chlorophenyl)-2-indolin-N-ylpropanamide

In an oven-dried flask under an atmosphere of nitrogen, 11.75 mL 1 M solution of (CH₃)₂AlCl in CH₂Cl₂ was added via addition funnel to a stirring suspension of 1.15 g (11.75 mmol) N,O-dimethylhydroxylamine hydrochloride at 0° C. After warming to room temperature a solution of 970 mg (2.94 mmol) of Ethyl 3-(4chlorophenyl)-2-indolinylpropanoate in 10 mL was added via addition funnel.

After stirring at room temperature for 5 h, 35 mL pH=8 phospate buffer solution was added and the resulting solution was stirred vigorously for 30 minutes. The phases were separated and the aqueous layer was extracted 2 times with chloroform. The combined organics were washed with water and then dried over MgSO₄. 965 mg (95%) of brown oil were collected. The crude material was carried on to the next step. ). TLC R_{f}=0.12 (10:1 hexanes:EtOAc). ¹H NMR (500 MHz, CDCl₃): δ 2.83 (m, 1H), 2.97(m, 2H), 3.13 (s, 3H), 3.34 (m, 1H), 3.45 (s, 3H), 3.61 (m, 2H), 4.87 (b, 1H), 6.54 (d, 1H), 6.66 (t, J=7.1 Hz, 1H), 7.07 (t, J=7.1 Hz, 2H), 7.18 (d, J=8.5 Hz, 2H), 7.24 (d, J=8.5 Hz, 2

### Step C: 4-(4-chlorophenyl)-3-indolin-N-ylbutan-2-one

In an oven dried flask under an atmosphere of nitrogen, 2.8 mL 1 M solution of CH₃MgBr in THF was added dropwise to a stirring solution of N,O-dimethyl-3-(4-chlorophenyl)-2-indolinylpropanamide in 25 mL anhydrous THF. The solution was stirred for 4 h while being allowed to warm to room temperature. Then approximately 20 mL water were added. The solution was extract three times with 50 mL ether. The combined extracts were dried over MgSO₄. The solvents were removed under reduced pressure yielding a brown oil which was carried on to the next step without purification. LC/MS m/e=301 (M+1). TLC R_{f}=0.5 (4:1 hexanes:EtOAc). ¹H NMR (500 MHz, CDCl₃): δ 2.14 (s, 3H), 2.81 (dd, J=14.6, 6.6 Hz, 1H), 2.97 (t, J=8.5 Hz, 2H), 3.26 (m, 2H), 3.5 (m, 1H), 4.21 (dd, J=6.6, 6.6 Hz), 6.39 (d, J=8 Hz, 1H), 6.66 (dd, J=7, 7 Hz, 1H), 7.07 (m, 2H), 7.13 (d, J=8.5 Hz), 7.22 (d, J=8.3 Hz).

### Step D: 4-(4-chlorophenyl)-3-indolin-N-ylbutan-2-one methoxime

A solution of 472 mg (1.573 mmol) of the product of Step C and 263 mg (3.147 mmol) of methoxylamine hydrochloride in anhydrous ethanol was treated with 255 μL (3.147 mmol) of pyridine. The solution was stirred for 2 h at room temperature. Solvent was removed under reduced pressure and the residue was partitioned between water and ether. The water was extracted with ether again. The extracts were then combined and dried over MgSO₄, filtered and concentrate to obtain crude material. obtained. Both the E and Z isomers were carried onto the next step. LC/MS m/e=330 (M+1). TLC R_{f}=0.77 and 0.65 (4:1 hexanes:EtOAc). ¹H NMR (500 MHz, CDCl₃): δ 1.78 (2s, 1H), 2.88 (dd, J=6.2, 13.8 Hz, 1H), 2.95 (m, 2H), 3.30 (m, 2H), 3.45 (m, 1H), 3.75 and 3.89 (2s, 3H), 4.21 (dd, J=6.9, 7.8 Hz, 1H), 6.28 and 6.47 (2d, J=8.1, 1H), 6.61 (m, 1H), 7.02 (m, 2H), 7.22 (m, 4H).

### Step E: 2-Amino-3-indolin-N-yl-4(4-chloro)phenylbutane

In an oven-dried flask equipped with a water condenser under an atmosphere of nitrogen, a solution of 301 mg (0.914 mmol) 4-(4-chlorophenyl)-3-indolinylbutan-2-one methoxime in 1.5 mL anhydrous THF was treated with 3.7 mL (3.7 mmol) of 1M BH₃.THF at room temperature. The solution was then heated to 75° C. for 2 days. The solution was then cooled to 0° C. and treated with chips of ice until bubbling subsided. 500 μL of 20% KOH were then added and the solution was heated at 45° C. for 2 h. The solution was then cooled to room temperature and extracted with ether 3×. The combined extracts were dried over MgSO₄, filtered, and concentrated to afford crude amine which was used in the next experiment without further purification. LC/MS m/e=302 (M+1). ¹H NMR (500 MHz, CDCl₃): δ 1.13, 1.14 (2d, J=6.5 Hz, 1H), 1.55-1.60 (m, 2H), 2.80-3.10 (m, 4H), 3.30-3.60 (m, 2H), 6.348 and 6.38 (2d, J=7.9 Hz, 1H), 6.50-6.78 (m, 2H), 6.95-7.24 (m, 5H)

### REFERENCE EXAMPLE 58

### 2-Amino-3-indol-N-yl-4(4-chloro)phenylbutane

This compound was prepared in an analogous manner to Reference Example 57 except that during Step A, sodium hydride was used as the base instead of the lithium hydroxide monohydrate/molecular sieves combination and indole was substitued for indoline. LCIMS: calculated for C₁₈H₁₉ClN₂ 299. observed m/e 300 (M+H)⁺(2.4 min).

### REFERENCE EXAMPLE 59

### 2-Amino-3-(N-methyl,N-phenyl)amino-4(4-chloro)phenylbutane

This compound was prepared in an analogous manner to Reference Example 57, substituting N-methylaniline for indoline in Step A. LC/MS: calculated for C₁₇H₂₁ClN₂ 289. observed m/e 290 (M+H)⁺(2.4 min).

### REFERENCE EXAMPLE 60

### 2-Amino-3-(7-azaindol-N-yl)-4(4-chloro)phenylbutane

This compound was prepared in an analogous manner to Reference Example 57, substituting 7-aza-indole for indole in Step A. LC/MS: calculated for C₁₇H₁₈ClN₃ 300. observed m/e 301 (M+H)⁺(2.7 min).

### REFERENCE EXAMPLE 61

### 2-Amino-3-(benzisoxazol-3-yl)-4(4-chloro)phenylbutane

This compound was prepared in an analogous manner to Reference Example 57 except starting with ethyl (benzisoxazol-3-yl)acetate in Step B. LC/MS: calculated for C₁₇H₁₇ClN₂O 300. observed m/e 301 (M+H)⁺(2.2 min).

### REFERENCE EXAMPLE 62

### 4-(4-Methylphenyl)-3-phenylbutan-2-amine (mixture of 4 isomers)

### Step A: 1-Phenylacetone

To a solution of N-methyl-N-methoxyacetamide (9.9 mL. 97 mmol) in ether (300 mL) at 0° C. was added benzylmagnesium chloride (97 mL a 1M solution in ether). The cloudy, white reaction mixture was warmed to room temperature for 2 h and then quenched by careful addition of IN hydrochloric acid (100 mL). The organic phase was separated, washed with brine, dried over MgSO₄ and concentrated. The crude material was purified by column chromatography on silica gel eluting from 0-10% EtOAc/hexane to give the title compound. ¹H NMR (500 MHz, CDCl₃): δ 7.36 (t, J=7.1 Hz, 2H), 7.30 (t, J=7.3 Hz, 1H), 7.24 (d, J=7.3 Hz, 2H), 3.72 (s, 2H), 2.18 (s, 3H). LC-MS: m/e 135 (M+H)⁺ (1.95 min).

### Step B: 4-(4-Methylphenyl)-3-phenylbutan-2-one

1-Phenylacetone (200 mg, 1.49 mmol) was mixed with powdered potassium hydroxide (167 mg, 2.98 mmol) and tetra-n-butylammonium bromide (1 mol %, 5 mg) in a flask without solvent. This mixture was stirred at room temperature for 90 min. before the addition of 1-(chloromethyl)-4-methylbenzene (198 μl, 1.49 mmol). The reaction mixture was then stirred overnight before diluting with water and CH₂Cl₂. The aqueous layer was separated and neutralized to pH 7 with 2N hydrochloric acid and extracted again into CH₂Cl₂. The combined organic washes were dried with MgSO₄ and concentrated. The crude material was purified by column chromatography on silica gel eluting from 0-10% EtOAc/hexane to give the title compound. ¹H NMR (500 MHz, CDCl₃): δ 7.35 (t, J=7.0 Hz, 2H), 7.29 (t, J=7.4 Hz, 1H), 7.23 (d, J=7.1 Hz, 2H), 7.05 (d, 7.8 Hz, 2H), 6.98 (d, J=7.8 Hz, 2H), 3.94 (t, J=7.3 Hz, 1H), 3.43 (dd, J=13.9, 7.5 Hz, 1H), 2.91 (dd, J=14, 7.1 Hz, 1H), 2.32 (s, 3H), 2.08 (s, 3H). LC-MS: m/e 239 (M+H)⁺(3.61 min).

### Step C: 4-(4-Methylphenyl)-3-phenylbutan-2-amine

To a solution of the 4-(4-methylphenyl)-3-phenylbutan-2-one (308 mg, 1.29 mmol) in 7M ammonia in MeOH (5 mL) and acetic acid (3 mL) was added sodium cyanoborohydride (130 mg, 2.06 mmol) and the reaction stirred at room temperature overnight. The reaction was quenched by pouring into 2M sodium carbonate solution and extracted into EtOAc. The aqueous layer was salted and re-extracted. The combined organic extracts were dried over MgSO₄ and concentrated to give the title compound as a mixture of 4 isomers which was used without further purification. LC-MS: m/e 240 (M+H)⁺(2.22 min).

### REFERENCE EXAMPLE 63

### 4-(4-Methoxyphenyl)-3-phenylbutan-2-amine

Prepared using the procedures described in Example 62, Steps A-C, using 1-(chloromethyl)-4-methoxybenzene as the alkylating agent in Step B. LC-MS: m/e 256 (M+H)⁺(1.90 and 2.03 min).

### REFERENCE EXAMPLE 64

### 3-[2-Amino-1-(4-fluorobenzyl)propyl]benzonitrile

Prepared using the procedures described in Example 10 using 3-2-oxopropyl)benzonitrite and 1-(chloromethyl)-4-fluorobenzene as the reactants in Step B. LC-MS: m/e 269 (M+H)⁺(2.87 min).

### REFERENCE EXAMPLE 65

### N-[2-Phenyl-3-(4-fluorophenyl)-1-methylpropyl]amine hydrochloride (Diastereomer α)

The title compound was obtained by the method described in Reference Example 26, substituting 4-fluorobenzyl bromide for isobutyl iodide. LC-MS, Rₜ=2.2 min, m/e=244.

### REFERENCE EXAMPLE 66

### 2-(2,3-Dihydro-1-H-indol-1-yl)-1,4-dimethylpentylamine

### Step A: Ethyl (2-(2,3-dihydro-1H-indol-1-yl)-4-methylpentanoate

A solution of 0.53 g (3.3 mmol) of ethyl (S)-2-hydroxyisocaproate in 8 mL dry CH₂Cl₂was cooled in a −78° C. bath and 0.73 mL (4.34 mmol) of triflic anhydride and 0.6 mL (5.36 mmol) of 2,6 lutidine were added. After 15 min 2 mL (11.5 mmol) of diisopropylethylarnine was added and stirred for 10 min. To this solution 0.36 mL (3.21 mmol) of 2,3-dihydroindoline was added and stirred overnight as it slowly warmed to room temperature. The reaction was quenched with saturated NaHCO₃ solution and extracted with ether. The combined organic layer was washed with water, brine, dried and concentrated. The residue was purified on a flash column using a gradient of 5-10% EtOAc/hexane to isolate the title compound. ¹H NMR: (500 MHz, CDCl₃): δ 0.99 (d, 3H), 1.03 (d, 3H), 1.22 (t, 3H), 1.81 (m, 3H), 3.04 (m, 2H), 3.57 (m, 1H), 3.66 (m, 1H), 4.14 (q, 2H), 4.24 (t, 1H), 6.4-7.1 (m, 4H).

### Step B: 3-(2,3-Dihydro-1H-indol-1-yl)-5-methylhexan-2-one

To a solution of 0.54 g (2.07 mmol) of ethyl (2-(2,3-dihydro-1H-indol-1-yl)-4-methylpentanoate in 10 mL CH₂Cl₂, 1.98 g (10 mmol) of N,O-dimethylhydroxylamine hydrochloride and 1.4 mL triethylamine were added. The mixture was cooled in an ice bath and 10 mL (10 mmol) 1 M diethylaluminium chloride in toluene was added. The reaction was stirred overnight as it warmed to room temperature then carefully quenched by pouring into 1.2 N HCl. The solution was extracted with CH₂Cl₂. The organic layer was washed with brine, dried and concentrated leaving amide which was used without purification. This amide was dissolved in 5 mL THF and 2.5 mL (3.5 mmol) of 1.4 M methylmagnesium bromide was added. After 1 h, the solution was quenched with 1.2 N HCl and extraced with EtOAc. The EtOAc layer was washed with brine, dried and concentrated. The residue was chromatographed using a gradient of 5-10% EtOAc-hexane to isolate the title compound. ¹H NMR: (500 MHz, CDCl₃): δ 0.96 (d, 3H), 0.99 (d, 3H), 1.7 (m, 3H), 2.17 (s, 3H), 3.06 (m, 2H), 3.04 (q, 1H), 3.52 (m, 1H), 4.11 (m, 1H) 6.4-7.1 (m, 4H).

### Step C: 2-(2,3-Dihydro-1-H-indol-1-yl)-1,4-dimethylpetylamine

To a solution of 0.185 g (0.8 mmol) of 3-(2,3-dihydro-1H-indol-1-yl)-5-methylhexan-2-one in 2 mL ethanol, 0.135 g O-methylhydroxylamine hydrochloride and 0.13 mL (1.6 mmol) of pyridine were added. After stirring for 2 h, the solution was concentrated and the residue was partitioned between water and EtOAc. The organic layer was washed with brine, dried and concentrated to give 0.2 g O-methyloxime as a mixture of isomers. This mixture was dissolved in 2 mLTHF and 1.5 mL 1 M BH₃ in THF was added. After gas evolution ceased, the reaction was heated in a 50° C. bath. After 2 h another 1.5 mL 1 M BH₃ in THF was added and heating was continued overnight. The reaction mixture was cooled and quenched with MeOH and concentrated. The residue was dissolved in 6 mL CH₂Cl₂ and 2 mL 1 N NaOH was added. After stirring for 15 min the layers were separated and the aqueous layer was extracted with CH₂Cl₂. The combined organic layer was washed with water, brine dried and concentrated to isolate title compound as a mixture of diastereomers which was used without purification. LC-MS, Rₜ=2.24 min, m/e=233.

The following amines were synthesized by the method of Reference Example 66.

### REFERENCE EXAMPLE 67

### 3-Cyclobutyl-2-(3,4-dihydroquinoline-1(2H)-yl)-1-methylpropylamine

LC-MS, Rₜ=2., 8 min, m/e=259.

### REFERENCE EXAMPLE 68

### 2-(3,4-Dihydroquinoline-1(2H)-yl)-1,4-dimethylpentylamine

LC-MS, Rₜ=2.74 min, m/e=248.

### REFERENCE EXAMPLE 69

### 2-(1H-1,2,3-Benzotriazol-1-yl)-3-(4-chlorophenyl)-1-methylpropylamine

### Step A: 2-(1H-1,2,3-Benzotriazol-1-yl)-N-methoxy-N-methylacetamide

A mixture of 1.77 g (10 mmol) of 2-(1H-1,2,3-benzotriazol-1-yl)acetic acid, 1.07 g (11 mmoles) of N,O-dimethylhydroxylamine hydrochloride, 5.8 g (11 mmol) of PyBOP, and 3.4 mL (24.2 mmol) of diisopropylethylamine in 50 mL CH₂Cl₂ was stirred overnight at RT. This mixture was partitioned between EtOAc and water. The organic layer was washed with brine and dried over anhydrous MgSO₄. Solvent removal afforded a crude product which was purified on silica gel using 60% EtOAC in hexane as solvent to give 2.01 g the desired amide as a solid. ¹H NMR: (CDCl₃): δ 3.26 (s, 3H), 3.84 (s, 3H), 5.63 (s, 2H), 7.35-8.2 (m, 4H).

### Step B: 2-(1H-1,2,3-Benzotriazol-1-yl)-3-(4-chlorophenyl)-N-methoxy-N-methyl-propanamide

To a solution of 2.0 g (9 mmol) of 2-(1H-1,2,3-benzotriazol-1-yl)-N-methoxy-N-methylacetamide in 15 mL anhydrous THF at −78° C., 10 mL (10 mmol) of 1M lithium bis(trimethylsilyl)amide was added dropwise. After stirring for 25 min, a solution of 2.06 g (10 mmol) of 4-chlorobenzyl bromide in 2 mL anhydrous THF was added. The resulting reaction mixture was allowed to warm to RT and stirred for 6 h. This reaction was quenched, diluted with 75 mL EtOAc and washed 3 times with 10 mL each of brine. After drying the organic phase solvent removal afforded a crude product which was purified on silica gel using 40% EtOAc in hexane as solvent to afford the desired product as a solid. ¹H NMR: (CDCl₃): δ 3.2 (s, 3H), 3.34 (s, 3H), 3.52 (m, 1H), 3.7 (m, 1H), 6.32 (t, 1H), 6.9-8.2 (m, 8H).

### Step C: 2-(1H-1,2,3-Benzotriazol-1-yl)-3-(4-chlorophenyl)-butan-2-one

To a solution of 1.73 g (5 mmol) of 2-(1H-1,2,3-benzotriazol-1-yl)-3-(4-chlorophenyl)-N-methoxy-N-methyl-propanamide in 10 mL anhydrous THF at 0° C., 4 mL (10 mmol) of 2.5M methyl magnesium bromide in ether was added. The reaction mixture was stirred for 4 h as it warmed to RT. The reaction was quenched by adding 10 mL 1N HCl and the resulting mixture was partitioned between EtOAc and water. The organic phase was washed with brine and dried over anhydrous MgSO₄. Solvent removal gave a crude ketone, which was purified on silica gel using 40% EtOAc in hexane to provide the desired ketone.

### Step D: 2-(1H-1,2,3-Benzotriazol-1-yl)-3-(4-chlorophenyl)-1-methyl propylamine

To a solution of 1.18 g (4 mmol) of 2-(1H-1,2,3-benzotriazol-1-yl)-3-(4-chlorophenyl)-butan-2-one in 8.5 mL (60 mmol) of 7N ammonia in MeOH at 0° C., 4 mL (964 mmol) of glacial acetic acid was added followed by 410 mg (6.5 mmol) of sodium cyanoborohydride. The reaction mixture was allowed to warm to RT and stirred overnight. The reaction was partitioned between EtOAc and saturated NaHCO₃ solution. The organic phase was dried over anhydrous MgSO₄. The solvent was removed in vacuo and the residue was purified on silica gel using a mixture of 5% 2N methanolic ammonia solution and 95% CH₂Cl₂to give the desired amine as a mixture of diastereomers. LC-MS, Rₜ=2.0 min, m/e=301.

### REFERENCE EXAMPLE 70

### 3-(4-Chlorophenyl)-2-(thiophene-3-yl)-1-methylpropylamine

The title amine was prepared by the method described in Reference Example 69, substituting thiophene-3-acetic acid for 2-(1H-1,2,3-benzotriazol-1-yl)acetic acid in Step A. LC-MS, Rₜ=2.19 min, m/e=266.

### REFERENCE EXAMPLE 71

### 3-(4-Chlorophenyl)-2-(thiophene-2-yl)-1-methylpropylamine

### Step A: 3-(4-Chlorophenyl)-2-(thiophen-2-yl)-butan-2-one

The title compound was obtained from 2-thiopheneacetic acid according to the procedure described in Reference Example 10, Steps A-D.

### Step B: 3-(4-Chlorophenyl)-2-thiophene-2-yl)-1-methylpropylamine

This amine was synthesized by the method of Reference Example 69, Step D. LC-MS, Rₜ=2.18 min, m/e=266.

### REFERENCE EXAMPLE 72

### 3-(4-Chlorophenyl)-1-methyl-2-(1-methyl-1H-indol-3-yl)propylamine

The title compound was prepared according to the method described in Reference Example 69. LC-MS: Rₜ=2.5 min, m/e=313.

### REFERENCE EXAMPLE 73

### 3-4-Chlorophenyl)-1-methyl-2-(1H-indazol-1-yl)propylamine

### Step A: 3-(4-Chlorophenyl)-2-(1H-indazol-1-yl)-butan-2-one

The title compound was obtained from indazol-1-yl-acetic acid by following the procedure of Reference Example 10, Steps A-D.

### Step B: 3-(4-Chlorophenyl)-1-methyl-2-(1H-indazol-1-yl)propylamine

The title amine was prepared according to the procedure of Reference Example 69, Step D. LC-MS: Rt=2.24 min, m/e=300.

### REFERENCE EXAMPLE 74

### 3-(4-Chlorophenyl)-1-methyl-2-(1-methyl-1H-indol-4-yl)propylamine

### Step A: 4-Chloro-1-methylindole

In a 100 mL flask, 0.3 g (7.5 mmol) sodium hydride was washed twice with dry hexane. The solid was suspended in 15 mL dry THF and 1 g (6.6 mmol) 4-chloroindole was drop wise added. After 15 min, 0.5 rnL (7.9 mmol) methyl iodide was added and the solution was stirred overnight. The reaction was quenched with 1.2 N HCl and partitioned between ether and water. The organic layer was washed with brine, dried and concentrated keeping the bath temperature below 30° C. The residue was purified on a flash column using a gradient of 5-10% EtOAc/hexane to isolate the desired product. ¹H NMR: (500 MHz, CDCl₃): δ 3.84 (s, 3H), 6.63 (d, 1H), 7-7.3 (m, 4H).

### Step B: 1-(1-Methyl-1H-indol-4-yl)acetone

To a solution of 0.852 g (5.14 mmol) of 4-chloro-1-methylindole in 15 mL dry toluene, 0.85 mL (7.73 mmol) isopropenyl acetate and 2.3 mL (8 mmol) tributyltin methoxide were added. The solution was heated to 100° C. After 15 min, 0.24 g (0.61 mmol) 2-dicyclohexylphospino-2′-(N,N-dimethylamino) biphenyl and 0.14 g (0.153 mmol) tris (dibenzylidineacetone)dipalladium were added and heating was continued. After 2 h the solution was cooled, filtered through a pad of CELITE diatomaceous earth and the filtrate was concentrated to ca. 5 mL. This solution was purified on a silica column using a gradient of 5-20% EtOAc/hexane to obtain the title compound. ¹H NMR: (500 MHz, CDCl₃): δ 2.14 (s, 3H), 3.84 (s, 3H), 3.97 (s, 2H), 6.51 (d, 1H), 7-7.3 (m, 4H).

### Step C: 4-(4-Chlorophenyl)-3-(1-methyl-1H-indol-4-yl)-butan-2-one

To a suspension of 135 mg (3.38 mmol) of sodium hydride in 8 mL dry THF, a solution of 605 mg (3.23 mmol) 1-(1-methyl-1H-indol-4-yl)acetone in 2 mL THF was added. The mixture was stirred for 45 min during which time the sodium hydride dissolved and a yellow orange solution resulted. The reaction was cooled in ice bath and 660 mg (3.24 mmol) 4-chlorobenzyl bromide in 1 mL THF was added. The cold bath was removed and the solution was stirred for 1.5 h. The reaction was quenched with 1.2 N HCl and extracted with EtOAc. The organic layer was washed with brine, dried and concentrated. The residue was chromatographed using a gradient of 10-20% EtOAc/hexane to isolate the desired product. ¹H NMR: (500 MHz, CDCl₃): δ 2.03 (s, 3H), 3.07 (m, 1H), 3.58 (m, 1H), 3.84 (s, 3H), 4.23 (t, 1H), 6.52 (d, 1H), 6.9-7.3 (m, 8H).

### Step D: 3-(4-Chlorophenyl)-1-methyl-2-(1-methyl-1H-indol-4-yl)propylamine

The title compound was prepared from 4-(4-chlorophenyl)-3-(1-methyl-1H-indol-4-yl)-butan-2-one by following the procedure of Reference Example 69, Step D. LC-MS, Rₜ=2.4 min, m/e=313.

### REFERENCE EXAMPLE 75

### 3-(4-Chlorophenyl)-1-methyl-2-(pyridazin-3-yl)propylamine cl Step A: 4-(4-Chlorophenyl)-3-(pyridazin-3-yl)-butan-2-one

This compound was synthesized from 3-iodopyridazine by the procedure of Reference Example 42, Steps A-D.

### Step B: N-2,4-Dimethoxybenzyl-N(3-(4-chlorophenyl)-1-methyl-2-(pyridazin-3-yl)propyl)amine

A solution of 300 mg (1.15 mmol) 4-(4-chlorophenyl)-3-(pyridazin-3-yl)-butan-2-one in 4 mL dichloroethane was treated with 234 mg (1.15 mmol) 2,4-dimethoxybenzyl amine hydrochloride, 0.16 mL (1.15 mmol) triethylamine and 488 mg (2.3 mmol) sodium triacetoxyborohydride. After stirring the reaction overnight, it was partitioned between water and CH₂Cl₂. The organic layer was washed with brine, dried and concentrated and the residue was purified on a flash column using 3% MeOH—CH₂Cl₂to isolate the desired amine.

### Step C: 3-(4-Chlorophenyl)-1-methyl-2-(pyridazin-3-yl)propylamine

A solution of 300 mg N-2,4-dimethoxybenzyl-N(3-(4-chlorophenyl)-1-methyl-2-(pyridazin-3-yl)propyl)amine in 5 mL trifluoroacetic acid was heated in a 70° C. bath over night followed by 6 h in a 100° C. bath. The reaction was cooled, concentrated and the residue was diluted with EtOAc. This solution was quenched (to pH 10) with 1N NaOH and the layers were separated. The organic layer was washed with brine, dried and concentrated. The residue was purified on a prepTLC using 10% MeOH/CH₂Cl₂with 1% NH₄OH to isolate the title compound (mnixture of diastereomers), starting material was also recovered. LC-MS, Rt=1.63 min, m/e=262.

### REFERENCE EXAMPLE 76

### 3-(4-Chlorophenyl)-1-methyl-2-(pyrimidin-5-yl)propylamine

### Step A: 4-(4-Chlorophenyl)-3(pyrimidin-5-yl)-butan-2-one

The title compound was obtained from 5-bromopyrimidine following the method of Reference Example 75, Steps A-C except that 2-(di-t-butylphosphino)biphenyl was used in place of dicyclohexylphospino-2′-(N,N-dimethylamino)biphenyl in Step B.

### Step B: 3-(4-Chlorophenyl)-1-methyl-2-(pyrinidin-5-yl)propylamine

The title compound was prepared by the procedure described in Reference Example 10, Steps E-I. LC-MS, Rt=1.57 min, m/e=262.

### REFERENCE EXAMPLE 77

### 2-(3-Cyanophenyl)-3-cyclobutyl-1-methylpropylamine

### Step A: 1-(3-Cyanophenyl)acetone

The title compound was prepared from 3-bromobenzonitrile and isopropenyl acetate by the procedure of Reference Example 42, Step A.

### Step B: 3-(3-Cyanophenyl)-4-cyclobutyl-butan-2-one

To a solution of 1.45 g (9.07 mmol) of 1-(3-cyanophenyl)acetone in 18 mL acetonitrile, 1.1 mL (9.5 mmol) cyclobutyl bromide and 5.91 g (18.1 mmol) cesium carbonate were added. After heating the solution in a 60° C. bath overnight, it was cooled and filtered. The filtrate was partitioned between water and EtOAc and the aqueous layer was extracted with EtOAc. The combined organic layer was washed with brine, dried and concentrated. The residue was purified on a flash column using a gradient of 5-10% EtOAc/hexane to isolate the title compound. ¹H NMR: (500 MHz, CDCl₃): δ 1.5-2.2 (m, 9H), 2.13 (s, 3H), 3.64 (m, 1H), 7.4-7.7 (m, 4H).

### Step C: 2-(3-Cyanophenyl)-3-cyclobutyl-1-methylpropylamine

This amine was prepared by following the method of Reference Example 10, Steps E-I. LC-MS, Rt=2.48 min, m/e=229.

The compounds of Reference Examples 78-80 were obtained by procedures described in Reference Example 77.

### REFERENCE EXAMPLE 78

### 2-(3-Cyanophenyl)-3-cyclopropyl-1-methylpropylamine

LC-MS, Rt=1.8 min, m/e=215.

### REFERENCE EXAMPLE 79

### 2-(3-Cyanophenyl)-3-cyclopentyl-1-methylpropylamine

LC-MS, Rt=2.7 min, m/e=243.

### REFERENCE EXAMPLE 80

### 2-3-Cyanophenyl)-3-cyclohexyl-1-methylpropylamine

LC-MS, Rt=2.8 min, m/e=257.

### REFERENCE EXAMPLE 81

### 2-3-(Cyanophenyl)-3-(1-tert-butyloxycarbonyl-piperidin-4-yl)-1-methylpropylamine

### Step A: 3-(3-Cyanophenyl)-4-(1-tert-butyloxycarbonyl-piperidin-4-yl)-butan-2-one

The title compound was synthesized by the method of Reference Example 77, Steps A-B.

### Step B: 2-(3-Cyanophenyl)-3-(1-tert-butoxycarbonyl-piperidin-4-yl)-1-methylpropylamine

The title amine was obtained by the method of Reference Example 10, steps E-G except that di-tert-butyl dicarbonate was not added in Step G. LC-MS, Rt=2.72 min, m/e=258 (M-99). (0.70 min).

### REFERENCE EXAMPLE 82

### N-[3-(4-Chlorophenyl)-2-(3-methylthiophenyl)-1-methylpropyl]amine hydrochloride (Diastereomer α)

The title compound was prepared following the same procedure as described in Example 42 substituting 3,5-dibromopyridine with 3-bromothioanisole at Step A. LC-MS: m/e 306 (M+H)⁺(2.68 min).

### REFERENCE EXAMPLE 83

### N-[3-(4-Chlorophenyl)-2-(2-chlorophenyl)-1-methylpropyl]amine

### Step A: 4-(4-Chlorophenyl)-3-(2-chlorophenyl)-butan-2-one

The title compound was prepared following the same procedure as described in Reference Example 17 Step A substituting phenylacetone with 2-chlorophenylacetone. ¹H NMR: (500 MHz, CDCl₃): δ 2.07 (s, 3H), 2.91 (dd, J=14, 6.9 Hz, 1H), 3.41 (dd, J=14, 6.9 Hz, 1H), 4.54 (t, J=7.2 Hz, 2H), 7.06-7.10 (m, 2H), 7.20-7.31 (m, 5H), 7.42-7.44 (m, 1H).

### Step B: N-[3-(4-Chlorophenyl)-2-(2-chlorophenyl)-1-methylpropyl]amine

The title compound was prepared following the same procedure as described in Reference Example 57 Steps D-E substituting 4-(4chlorophenyl)-3-indolin-N-ylbutan-2-one with 4-(4-chlorophenyl)-3-(2-chlorophenyl)-butan-2-one (from Step A). ¹H NMR: (500 MHz, CDCl₃): δ 1.05, 1.24 (d, J=6.4, 6.2 Hz, 3H), 1.42 (br s, 2H), 2.8-3.0 (m, 1H), 3.15-3.35 (m, 2H), 3.4-3.6 (m, 1H), 6.96-6.98 (m, 2H), 7.05-7.40 (m, 6H).

### REFERENCE EXAMPLE 84

### N-[3-(4-Chlorophenyl)-2-(6-chloroindol-N-yl)-1-methylpropyl]amine

### Step A: (N-Carboethoxymethyl)-6-chloroindole

6-Chloroindole (5.0 g, 33 mmol) was dissolved in anhydrous N,N-dimethylformamide (165 mL) in a 500 mL round bottom flask. Sodium hydride (1.71 g or 60% oil dispersion, 43 mmol) was added batchwise and the resulting mixture stirred at room temperature for 1 h. Subsequently, ethyl bromoacetate was added dropwise and the mixture allowed to stir at 30° C. overnight. Water (200 mL) and ethyl acetate (165 mL) were added and phases were separated. The aqueous phase was reextracted with ethyl acetate (2×165 mL). The organic layers were combined and washed with water (3×165 mL), brine, and dried over anhydrous magnesium sulfate. The crude material was purified via silica gel chromatography (2× Biotage 40M column), eluting with mixtures of hexane and ethyl acetate. This provided the title compound. TLC: Rf=0.25 (10:1 hexane:ethyl acetate); LC-MS, Rt=3.55 min, m/e=238 (M+1). ¹H NMR: (500 MHz, CDCl₃): δ 1.31 (t, J=7.1 Hz, 3H), 4.28 (q, J=7.2 Hz, 2H), 4.86 (s, 2H), 6.55 (d, J=3.2 Hz, 1H),7.15 (d, J=3.2 Hz, 1H), 7.21-7.22 (m, 2H), 7.65 (m, 1H).

### Step B: Ethyl 3(4-chlorophenyl)-2-(6-chloroindol-N-yl)propanoate

The title compound was prepared in 36% isolated yield following the same procedure as described in Reference Example 10 Step A substituting sodium hexamethyldisilazide with lithium hexamethyldisilazide (1M in THF), and methyl phenylacetate with (N-Carboethoxymethyl)-6-chloroindole (from Step A). ¹H NMR: (500 MHz, CDCl₃): δ 1.22 (t, J=7.1 Hz, 3H), 3.40 (m, 1H), 3.48 (m, 1H), 4.23 (q, J=7.2 Hz, 2H), 5.15 (m, 1H), 6.6 (d, J=3.2 Hz, 1H), 7.0 (m, 2H), 7.15-7.35 (m, 5H), 7.59 (m, 1H).

### Step C: 2-Amino-3-(6-chloroindol-N-yl)-4-(4-chloro)phenylbutane

The title compound was prepared following the same procedures as described in Reference Example 57 Step B through E substituting ethyl 3-(4-chlorophenyl)-2-indolin-N-ylpropanoate with ethyl 3(4-chlorophenyl)-2-(6chloroindol-N-yl)propanoate (from Step B). LC-MS, Rt=2.96 min, m/e=334 (M+1).

### REFERENCE EXAMPLE 85

### N-[3-(4-Chlorophenyl)-2-(5-chloroindol-N-yl)-1-methylpropyl]amine

The title compound was prepared following the same procedures as described in Reference Example 84 substituting 6-chloroindole with 5-chloroindole in Step A. LC-MS, Rt=3.02 min, m/e=334 (M+1).

### REFERENCE EXAMPLE 86

### N-[3-(4-Chlorophenyl)-2-(2-chloro)phenoxy-1-methylpropyl]amine

### Step A: 3-(4-Chlorophenyl)-2-(2chloro)phenoxypropanoic acid

The title compound was prepared following the same procedures as described in Reference Example 26 Step A substituting phenylacetic acid with (2-chloro)phenoxyacetic acid, and isobutyliodide with 4-chlorobenzyl bromide. ¹H NMR: (500 MHz, CDCl₃): δ 3.36 (d, J=2.8 Hz, 2H), 4.89 (dd, J=5.0, 6.4 Hz, 1H), 6.77 (dd, J=8.2, 0.9 Hz, 1H), 6.95 (dt, J=7.5, 1.1 Hz, 1H), 7.20 (dt, J=8.2, 1.6 Hz, 1H), 7.30-7.38 (m, 4H), 7.41 (dd, J=7.8, 1.6 Hz, 1H).

### Step B: N,O-Dimethyl-3-(4-chlorophenyl)-2-(2chloro)phenoxypropanamide

A mixture of 3-(4-Chlorophenyl)-2-(2chloro)phenoxypropanoic acid (620 mg, 1.99 mmol, from Step A), N-methoxy-N-methylamine hydrochloride (3 mmol, 300 mg), diisopropylethyl amine (776 mg, 1.05 mL, 6 mmole), N-(3-dimethylaminopropyl)-N′-ethylcarbodiimide (2.3 mmol 442 mg) in anhydrous dichloromethane (10 mL) was stirred at room temperature for 4 h. Water was added to quench the reaction. The organics were extracted with ethyl acetate 3 times. The organic extracts were combined and washed with 5% aqueous sodium bicarbonate (3 times), brine, and dried (anhydrous magnesium sulfate). The crude product obtained was purified via silica gel chromatography (Biotage 12M column), eluting with mixtures of hexanes and ethyl acetate. This provided the desired compound as a colorless oil. TLC: Rf=0.45 (1:1 hexane:ethylacetate). ¹H NMR: (500 MHz, CDCl₃): δ 3.23 (s, 3H), 3.25-2.40 (m, 2H), 3.43 (s, 3H), 5.15 (m, 1H), 6.82 (d, J=8.2 Hz, 1H), 6.95 (t, J=7.5 Hz, 1H), 7.15 (t, J=8.2 Hz, 1H), 7.30-7.42 (m, 5H).

### Step C: N-[3-(4-Chlorophenyl)-2-(2chloro)phenoxy-1-methylpropyl]amine

The title compound was prepared following the same procedures as described in Reference Example 57 Steps C-E substituting N,O-dimethyl-3-(4chlorophenyl)2-indolin-N-ylpropanamide with N,O-dimethyl-3-(4chlorophenyl)-2-(2-chloro)phenoxypropanamide (from Step B). ¹H NMR: (500 MHz, CDCl₃): δ 1.20, 1.26 (2s, 3H), 1.65 (br s, 2H), 2.85-3.25 (m, 3H), 4.29, 4.37 (2m, 1H), 6.67, 6.73 (2 dd, J=8.2, 1.1 Hz, 1H), 6.85-6.93(m, 1H), 7.11 (ddd J=8.0, 6.2, 1.6 Hz, 1H), 7.20-7.30 (m, 4H), 7.34-7.39 (m, 1H).

### REFERENCE EXAMPLE 87

### N-[3-(4-Chlorophenyl)-2-phenoxy-1-methylpropyl]amine

The title compound was prepared following the same procedures as described in Reference Example 86 substituting (2-chloro)phenoxyacetic acid with phenoxyacetic acid. Additionally, in Step A, 2 equivalents of lithium diisopropylamide was used instead of lithium hexamethyldisilazide. LC-MS, Rt=3.31 min, m/e=276 (M+1).

### REFERENCE EXAMPLE 88

### N-[3-(4-Chlorophenyl)-2-(4-chloro)phenoxy-1-methylpropyl]amine

The title compound was prepared following the same procedures as described in Reference Example 86 substituting (2-chloro)phenoxyacetic acid with (4-chloro)phenoxyacetic acid. ¹H NMR: (500 MHz, CDCl₃): δ 1.20, 1.22 (2s, 3H), 1.60 (br s, 2H), 2.87-3.25 (m, 3H), 4.20, 4.28 (2m, 1H), 6.74, 6.82 (m, 2H), 7.16-7.34 (m, 6H).

### REFERENCE EXAMPLE 89

### N-[3-(4-Chlorophenyl)-2-(4-bromo)phenoxy-1-methylpropyl]amine

The title compound was prepared following the same procedures as described in Reference Example 86 substituting (2chloro)phenoxyacetic acid with (4-bromo)phenoxyacetic acid. LC-MS, Rt=3.05 min, m/e=338, 340 (M+1).

### EXAMPLE 1

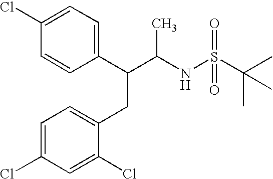

### N-[2-(4-Chlorophenyl)-3-(2,4-dichlorophenyl)-1-methylpropyl]-2-methyl-2-propanesulfonamide (3 isomers)

### Step A N-[3-(2,4-Dichlorophenyl)-2-(4-chorophenyl)-1-methylpopyl]-2-methyl-2-propanesulfinamide (3 isomers)

Formation of N-[3-(2,4-Dichlorophenyl)-2-(4-chorophenyl)-1-methylpropyl]-2-methyl-2-propanesulfinamide (3 isomers) is executed in accordance with Steps A-E of Reference Example 20.

### Step F: N-[3-(2,4-Dichlorophenyl)-2-(4-chorophenyl)-1-methylpropyl]-2-methyl-2-propanesulfonamide (3 isomers)

To a solution of N-[3-(2,4-dichlorophenyl)-2-(4-chorophenyl)-1-methylpropyl]-2-methyl-2-propanesulfinamide (faster eluting isomer, 10 mg, 0.023 mmol) in 0.5 mL of dichloromethane was added m-chloroperbenzoic acid (60%, 20 mg), and the mixture was stirred at room temperature for 1 h. The reaction mixture was loaded onto a silica gel column, and elution with 30% ether in hexane afforded the title compound. ¹H NMR (500 MHz, CD₃OD): δ 7.30 (d, 1 H), 7.22 (d, 2H), 7.08 (d, 2H), 7.00 (dd, 1H), 6.84 (d, 1H), 3.72 (m, 1H), 3.58 (dd, 1H), 3.04 (m, 1H), 2.93 (dd, 1H), 1.39 (s, 9H), 1.08 (d, 3H). LC-MS: m/e 448 (M+H)⁺(4.4 min).

The slower co-eluting isomers of N-[3-(2,4-dichlorophenyl)-2-(4-chorophenyl)-1-methylpropyl]-2-methyl-2-propanesulfinamide was converted to the title compounds using the same procedure as described above followed by HPLC purification on a Chiralcel OD column eluted with 5% ethanol in hexane to give two pure isomers.

Faster eluting isomer: Analytical HPLC: retention time=7.7 min (Chiralcel OD column, flow rate=0.75 mL/min, 5% ethanolfhexane). ¹H NMR (500 MHz, CD₃OD): δ 7.30 (d, 1 H), 7.22 (d, 2H), 7.08 (d, 2H), 7.00 (dd, 1H), 6.84 (d, 1H), 3.72 (m, 1H), 3.58 (dd, 1H), 3.04 (m, 1H), 2.93 (dd, 1H), 1.39 (s, 9H), 1.08 (d, 3H). LC-MS: m/e 448 (M+H)⁺(4.4 min).

Slower eluting isomer: Analytical HPLC: retention time=11.4 min (Chiralcel OD column, flow rate=0.75 mL/min, 5% ethanol/hexane). ¹H NMR (500 MHz, CD₃OD): δ 7.31 (d, 1 H), 7.22 (d, 2H), 7.18 (d, 2H), 7.00 (dd, 1H), 6.93 (d, 1H), 3.74 (m, 1H), 3.33 (dd, 1H), 3.20 (m, 1H), 3.05 (dd, 1H), 1.35 (d, 3H), 1.19 (s, 9H). LC-MS: m/e 448 (M+H)⁺(4.3 min).

### EXAMPLE 2

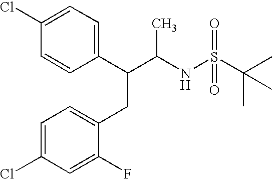

### N-[2-(4-Chlorophenyl)-3-(4-chloro-2-fluorophenyl)-1-methylpropyl]-2-methyl-2-propanesulfonamide (3 isomers)

The title compound was prepared following the same procedures of Example 1 substituting 2,5-dichlorobenzyl bromide with 4-chloro-2-fluorobenzyl bromide at Step A.

Isomer 1: LC-MS: m/e 454 (M+Na)⁺(4.2 min).

Isomer 2 and 3 (1:1, co-eluting on silica gel): LC-MS: m/e 454 (M+Na)⁺(4.2 min).

### EXAMPLE 3

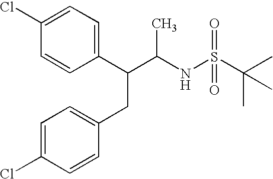

### N-[2,3-Bis(4-chlorophenyl)-1-methylpropyl]-2-methyl-2-propanesulfonamide (diastereomer α)

### Step A: N-[2,3-Bis(4-chlorophenyl)-1-methylpropyl]-2-methyl-2-propanesulfinamide (Diastereomer α)

To a suspension of 2-amino-3,4-bis(4-chlorophenyl)butane hydrochloride salt (Reference Example 29) (diastereomer α, 81 mg, 0.25 mmol) and diisopropylethylamine (0.13 mL, 0.74 mmol) in 1 mL of CH₂Cl₂ was added tert-butylsulfinyl chloride (70 mg, 0.49 mmol; prepared from tert-butylmagnesium chloride following the procedure of Weinreb, J. Org. Chem. 1997, 62, 8604). After stirring at room temperature for 2 h, the reaction mixture was loaded onto a silica gel column, which was eluted with 50% EtOAc in hexane to give the title compound as a mixture of diastereomers. LC-MS: m/e 398 (M+H)⁺(4.0 min).

### Step, B: N-[2,3-Bis(4-chlorophenyl)-1-methylpropyl]-2-methyl-2-propanesulfonamide (diastereomer α)

N-[2,3-Bis(4-chlorophenyl)-1-methylpropyl]-2-methyl-2-propanesulfinamide (Diastereomer α) was converted to the title compound following the procedure described in Example 1, Step F. ¹H NMR (500 MHz, CD₃OD): δ 7.22 (d, 2H), 7.12 (d, 2H), 7.08 (d, 2H), 6.95 (d, 2H), 3.64 (m, 1H), 3.41 (dd, 1H), 3.89 (m, 1H), 2.79 (dd, 1H), 1.18 (s, 9H), 1.04 (d, 3H). LC-MS: m/e 436 (M+Na)⁺(4.1 min).

Examples 4-9 (Table 1) were prepared following the procedures described in Example 3 substituting 2-amino-3,4-bis(4-chlorophenyl)butane hydrochloride salt with the appropriate amines from the Reference Examples. The diastereomer designations (α or β) correspond to designations of the starting amines.

TABLE 1
Compounds prepared according to the methods described in Examples 3.

| | | | | mass | eomer |
|---|---|---|---|---|---|
| Ex. | | | time | spectrum | α and/or |
| No. | Name | Structure | (min) | m/e | β |
| 4. | N-[2-3-Bis(4- chlorophenyl)-1- methylpropyl]-2-methyl-2- propanesulfonamide | 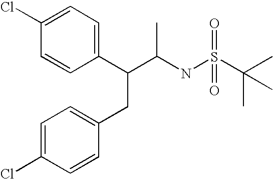 | 4.1 | 436 (M + Na)⁺ | β |
| 5. | N-[3-(4-Chlorophenyl)-2- phenyl-1-methylpropyl]-2- methyl-2- propanesulfonamide | 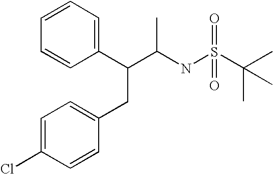 | 3.9 | 402 (M + Na)⁺ | β |
| 6. | N-[2,3-Diphenyl-1- methylpropyl]-2-methyl-2- propanesulfonamide | 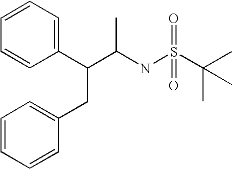 | 3.7 | 368 (M + Na)⁺ | α |
| 7. | N-[2-(4-Chlorophenyl)-3- phenyl-1-methylpropyl]-2- methyl-2- propanesulfonamide | 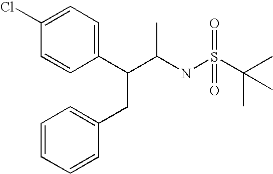 | 3.9 | 402 (M + Na)⁺ | α |
| 8. | N-[2-(4-Chlorophenyl)-3- phenyl-1-methylpropyl]-2- methyl-2- propanesulfonamide | 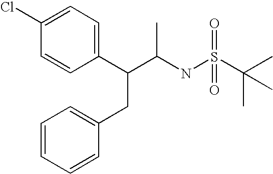 | 3.9 | 402 (M + Na)⁺ | β |
| 9. | N-[2,3-Diphenyl-1- methylpropyl]-2-methyl-2- propanesulfonamide | 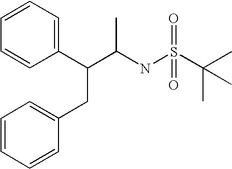 | 3.6 | 368 (M + Na)⁺ | β |

Examples 10-15 (Table 2) were prepared following the procedures described in Example 3 substituting tert-butylsulfinyl chloride with 1,1-dimethylphenethylsulfinyl chloride (prepared from 1,1-dimethylphenethylmagnesium chloride following the procedure as described for tert-butylsulfinyl chloride) and 2-amino-3,4-bis(4-chlorophenyl)butane hydrochloride salt with the appropriate amines from the Reference Examples. The diastereomer designations (α or β) correspond to designations of the starting amines.

TABLE 2
Compounds prepared according to the methods described in Example 3.

| | | | | HPLC- | Diaster- |
|---|---|---|---|---|---|
| | | | Retention | mass | eomer |
| Ex. | | | time | spectrum | α and/or |
| No. | Name | Structure | (min) | m/e | β |
| 10. | N-[2,3-Bis(4- chlorophenyl)-1- methylpropyl]-1,1- dimethylphenethylsulfon- amide | 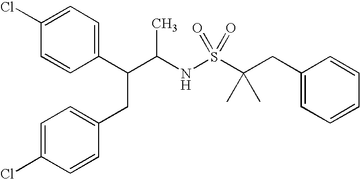 | 4.6 | 512 (M + Na)⁺ | α |
| 11. | N-[3-(4-Chlorophenyl)-2- phenyl-1-methylpropyl]- 1,1- dimethylphenethylsulfon- amide | 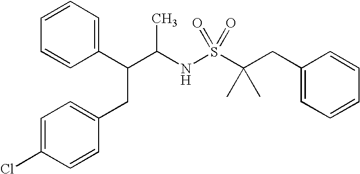 | 4.4 | 478 (M + Na)⁺ | α |
| 12. | N-[2-(4-Chlorophenyl)-3- phenyl-1-methylpropyl]- 1,1- dimethylphenethylsulfon- amide | 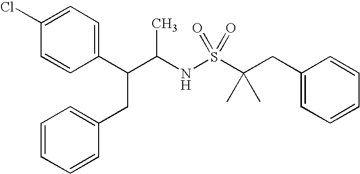 | 4.4 | 478 (M + Na)⁺ | α |
| 13. | N-[2,3-Diphenyl)-1- methylpropyl]-1,1- dimethylphenethylsulfon- amide | 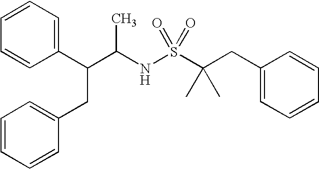 | 4.2 | 444 (M + Na)⁺ | α |
| 14. | N-[2,3-Bis-(4- chlorophenyl)-1- methylpropyl]-1,1- dimethylphenethylsulfon- amide | 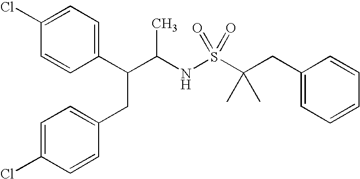 | 4.5 | 512 (M + Na)⁺ | β |
| 15. | N-[2-(4-Chlorophenyl)-3- (2,4-dichlorophenyl)-1- methylpropyl]-1,1- dimethylphenethylsulfon- amide | 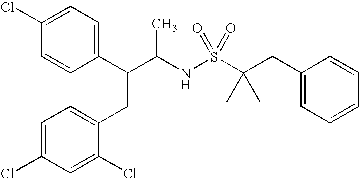 | 4.7 | 546 (M + Na)⁺ | Isomer 2 and 3 (see Ref Ex) |

### EXAMPLE 16

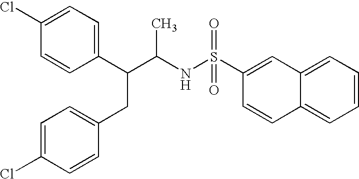

### N-[2,3-Bis(4-chlorophenyl)-1-methylpropyl]-2-naphthalenesulfonamide (diastereomer α)

To a suspension of 2-amino-3,4-bis(4-chlorophenyl)butane hydrochloride salt (Reference Example 1) (diastereomer α, 0.10 g, 0.30 mmol) and diisopropylethylamine (0.16 mL, 0.91 mmol) in 1 mL of CH₂Cl₂ was added 2-naphthalenesulfonyl chloride (0.10 g, 0.45 mmol). After stirring at room temperature overnight, the reaction mixture was loaded onto a silica gel column, which was eluted with 15% EtOAc in hexane to give the title compound. ¹H NMR (500 MHz, CD₃OD): δ 8.38 (d, 1H), 8.03 (d, 2H), 7.97 (d, 1H), 7.84 (dd, 1H), 7.65 (m, 2H), 7.17 (d, 2H), 7.08 (d, 2H), 7.03 (d, 2H), 6.88 (d, 2H), 3.51 (m, 1H), 3.29 (dd, 1H), 2.86 (m, 1H), 2.66 (dd, 1H), 0.68 (d, 3H). LC-MS: m/e 484 (M+H)⁺(4.4 min).

Examples 17-22 (Table 3) were prepared following the procedures described in Example 16 substituting 2-amino-3,4-bis(4chlorophenyl)butane hydrochloride salt with the appropriate amines from the Reference Examples. The diastereomer designations (α or β) correspond to designations of the starting amines.

TABLE 3
Compounds prepared according to the methods described in Example 16.

| | | | | HPLC- | |
|---|---|---|---|---|---|
| | | | retention | mass | Diaster- |
| Ex. | | | time | spectrum | eomer |
| No. | Name | Structure | (min) | m/e | α and/or β |
| 17. | N-[2,3-Bis(4-chlorophenyl)-1- methylpropyl]-2- naphthalenesulfonamide | 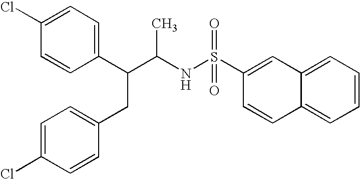 | 4.4 | 484 | β |
| 18. | N-[2-(4-Chlorophenyl)-3-(2,4- dichlorophenyl)-1- methylpropyl]-2- naphthalenesulfonamide | 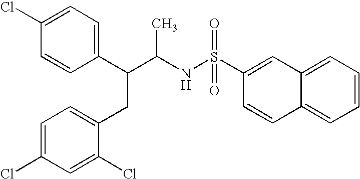 | 4.7 | 518 | Isomer 2 (faster on silica gel, see Ref Ex) |
| 19. | N-[2-(4-Chlorophenyl)-3-(2,4- dichlorophenyl)-1- methylpropyl]-2- naphthalenesulfonamide | 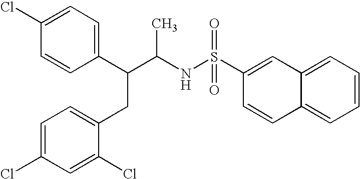 | 4.6 | 518 | Isomer 3 (slower on silica gel, see Ref Ex) |
| 20. | N-[3-(4-Chlorophenyl)-2- phenyl-1-methylpropyl]-2- naphthalenesulfonamide | 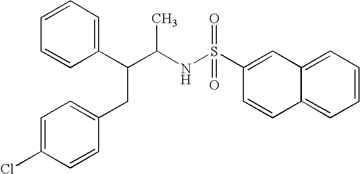 | 4.3 | 450 | α |
| 21. | N-[2-(4-Chlorophenyl)-2- phenyl-1-methylpropyl]-2- naphthalenesulfonamide | 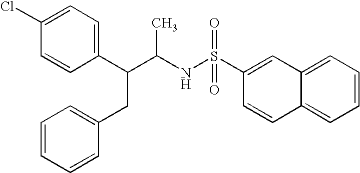 | 4.2 | 450 | α |
| 22. | N-[2,3-Diphenyl-1- methylpropyl]-2- naphthalenesulfonamide | 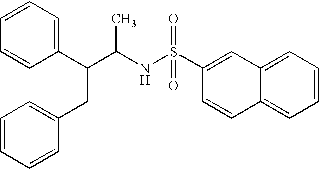 | 4.0 | 438 (M + Na)⁺ | α |

### EXAMPLE 23

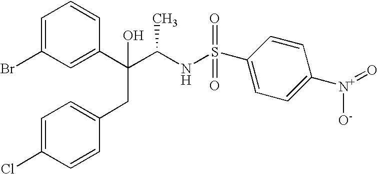

### N-[2-(3-Bromophenyl)-3-(4-chlorophenyl)-2-hydroxyl-1(S)-methylpropyl]-4-nitrobenzenesulfonamide

To a suspension of N-{[3-(4-chlorophenyl)-2-(3-bromophenyl)-2-hydroxyl-1(S)-methyl]propyl}amine hydrochloride (0.46 g, 1.2 mmol) in 5 mL of CH₂CH₂ was added N-methylmorpholine (0.66 mL, 6.0 mmol) and 4-nitrobenzenesulfonyl chloride (0.62 g, 2.8 mmol). After stirring at room temperature overnight, the reaction mixture was partitioned between ethyl acetate (20 mL) and water (20 mL). The organic layer was separated and the aqueous layer extracted with EtOAc (2×20 mL). The combined extracts were dried over anhydrous magnesium sulfate, filtered, and concentrated to dryness, and the residue was purified by flash column chromatography on silica gel eluting with 5 to 30% EtOAc in hexane to give the title compound. ¹H NMR (500 MHz, CD₃OD): δ 8.42 (d, 2H), 8.20 (d, 2H), 7.34 (t, 1H), 7.30 (d, 1H), 7.23 (d, 1H), 7.16 (t, 1H), 7.03 (d, 2H), 6.87 (d, 2H), 3.86 (q, 1H), 3.38 (d, 1H), 3.04 (d, 1H), 0.64 (d, 3H). LC-MS: m/e 539 (M+H)⁺(2.7 min).

### EXAMPLE 24

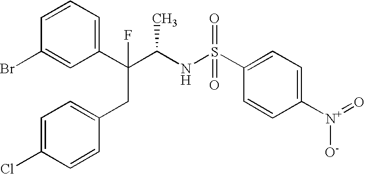

### N-[2-(3-Bromophenyl)-3-(4-chlorophenyl)-2-fluoro-1(S)-methylpropyl]-4-nitrobenzenesulfonamide

To a solution of N-[2-(3-bromophenyl)-3-(4-chlorophenyl)-2-hydroxyl-1(S)-methylpropyl]-4-nitrobenzenesulfonamide (Example 23, 0.24 g, 0.44 mmol) in 5 mL of methylene chloride was added (dimethylamino)sulfur trifluoride (0.20 mL, 1.6 mmol). After stirring overnight, the reaction was quenched by carefully transferring to a well-stirred saturated aqueous sodium bicarbonate (20 mL), and the product was extracted with ether (2×20 mL). The combined extracts were dried over anhydrous magnesium sulfate, filtered and concentrated to dryness, and the residue was purified on a silica gel column eluting with 5 to 40% ethyl acetate in hexane to give the title compound as one major diastereomer. ¹H NMR (500 MHz, CD₃OD): δ 8.42 (d, 2H), 8.19 (d, 2H), 7.08 (d, 2H), 6.87 (d, 2H), 3.96 (dq, 1H), 3.71 (dd, 1H), 3.09 (dd, 1H), 0.71 (d, 3H).

### EXAMPLE 25

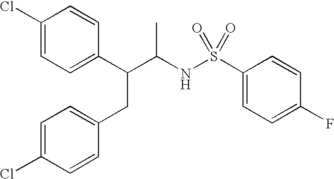

### N-[2,3-Bis-(4-chlorophenyl)-1-methylpropyl]-4-fluorobenzenesulfonamide

A mixture of 23 mg (0.075 mmol) of 2,3-bis-(4-chlorophenyl)-1-methylpropyamine hydrochloride, 33 mg (0.16 mmol) of p-fluorobenzenesulfonyl chloride, and 0.051 mL (0.3 mmol) of diisopropylethyl amine in 1 mL of dichloromethane was stirred overnight at room temperature. The resulting mixture was applied on 1000 micron silica gel plate, which was eluted with 15% ethyl acetate in hexane to isolate the title compound as an oil. ¹H NMR: 0.82 (3H; D, J=14 Hz); 2.80 (1H, m); 2.94(1H, m); 3.13(1H, m); 3.48 (1H, m); 4.02(1H, m); 6.5-7.8(12H. m). LC-MS: Retention time=4.2 min, m/e=474 (M+23).

Example 26-57 (Table 4) were prepared according to the procedures described in Example 25 substituting 2,3-bis(4-chlorophenyl)-1-methylpropylamine hydrochloride and p-fluorobenzenesulfonyl chloride with the appropriate amine and sulfonyl chloride.

TABLE 4
Compounds prepared according to the methods described in Example 25.

| | | | | HPLC- |
|---|---|---|---|---|
| | | | retention | mass |
| Ex. | | | time | spectrum |
| No. | Name | Structure | (min) | m/e |
| 26. | N-[2,3-bis-(4-chlorophenyl)- propyl]-benzenesulfonamide | 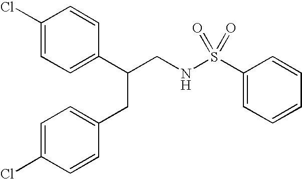 | 4.1 | 420 |
| 27. | N-[2,3-bis-(4-chlorophenyl)- propyl]-4- chlorobenzenesulfonamide | 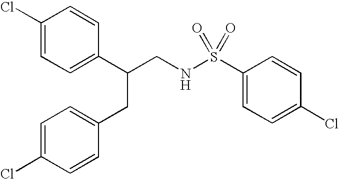 | | |
| 28. | N-[2,3-bis-(4-chlorophenyl)- propyl]-3- chlorobenzenesulfonamide | 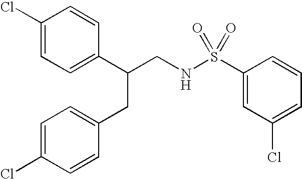 | 4.3 | 456 |
| 29. | N-[3-(4-chlorophenyl)-1- methyl-2-phenylpropyl]- benzenesulfonamide | 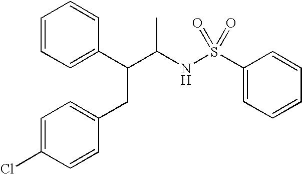 | 3.9 | 400 |
| 30. | N-[3-(4-chlorophenyl)-1- methyl-2-phenylpropyl]-4- chlorobenzenesulfonamide | 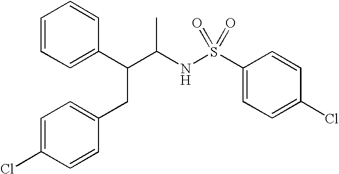 | 4.2 | 456 (M + 23) |
| 31. | N-[3-(4-chlorophenyl)-1- methyl-2-phenylpropyl]-4- fluorobenzenesulfonamide | 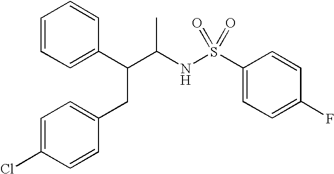 | 4.0 | 418 |
| 32. | N-[3-(4-chlorophenyl)-1- methyl-2-phenylpropyl]-3- chlorobenzenesulfonamide | 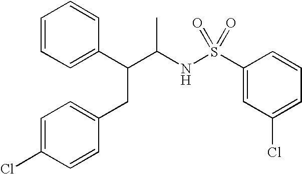 | 4.2 | 456 (M + 23) |
| 33. | N-[2,3-bis-(4-chlorophenyl)- 1-methylpropyl]- benzenesulfonamide | 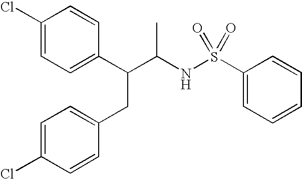 | 4.2 | 456 (M + 23) |
| 34. | N-[2,3-bis-(4-chlorophenyl)- 1-methylpropyl]-4- chlorobenzenesulfonamide | 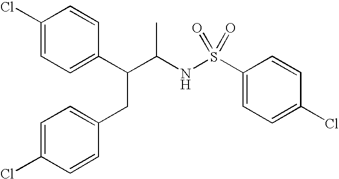 | 4.4 | |
| 35. | N-[2,3-bis-(4-chlorophenyl)- 1-methylpropyl]-3- chlorobenzenesulfonamide | 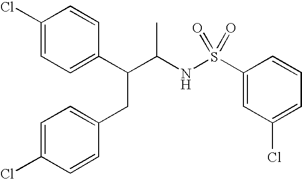 | 4.3 | 468 |
| 36. | N-[2,3-bis-(4-chlorophenyl)- propyl]-1- phenylmethanesulfonamide | 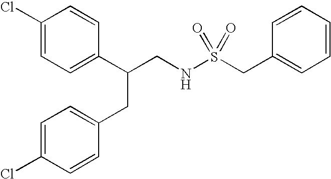 | 4.0 | 434 |
| 37. | N-[2,3-bis-(4-chlorophenyl)- 1-methylpropyl]-1- phenylmethanesulfonamide | 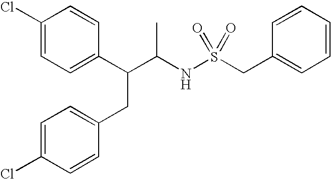 | 4.2 | 448 (M + 23) |
| 38. | N-[3-4-chlorophenyl)-1- methyl-2-phenylpropyl]-1- phenylmethanesulfonamide | 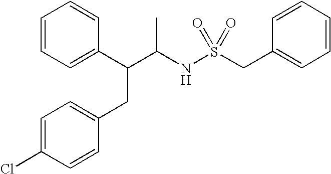 | 4.0 | 436 (M + 23) |
| 39. | N-[2,3-bis-(4-chlorophenyl)- 1-methylpropyl]-3,4- dichlorobenzenesulfonamide | 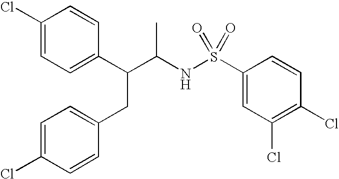 | 4.5 | 524 (M + 23) |
| 40. | N-[2,3-bis-(4-chlorophenyl)- 1-methylpropyl]-3,5- dichlorobenzenesulfonamide | 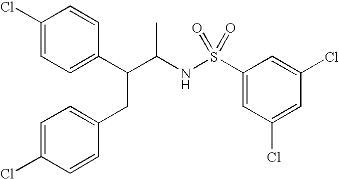 | 4.5 | 502 |
| 41. | N-[2,3-bis-(4-chlorophenyl)- 1-methylpropyl]-2,3,4- trichlorobenzenesulfonamide | 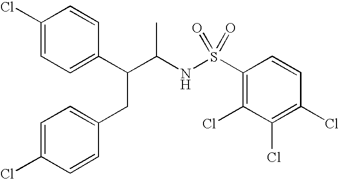 | 4.2 | 558 (M + 23) |
| 42. | N-[2,3-bis-(4-chlorophenyl)- propyl]-3,4- dichlorobenzenesulfonamide | 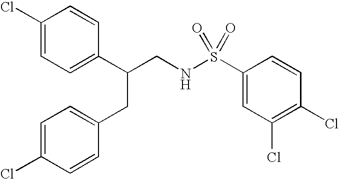 | 4.4 | 510 (M + 23) |
| 43. | N-[2,3-bis-(4-chlorophenyl)- propyl]-3,5- dichlorobenzenesulfonamide | 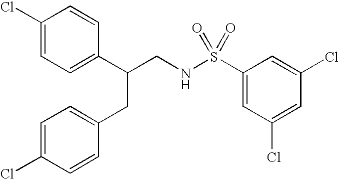 | 4.5 | 512 (M + 23) |
| 44. | N-[2,3-bis-(4-chlorophenyl)- propyl]-2,3,4- trichlorobenzenesulfonamide | 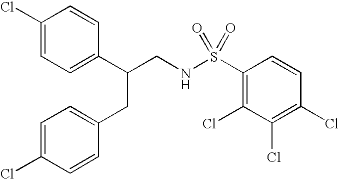 | 4.5 | 544 (M + 23) |
| 45. | N-[3-(4-chlorophenyl)-2-(3- bromophenyl)-1- methylpropyl]- benzenesulfonamide | 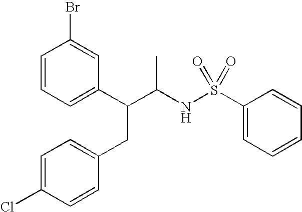 | 4.1 | 477/479 (M + 1) |
| 46. | N-[3-(4-chlorophenyl)-2-(2- chlorophenyl)-1- methylpropyl]- benzenesulfonamide | 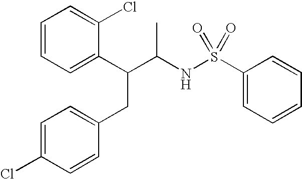 | 4.0 | 456 (M + 1) |
| 47. | N-[3-(4-chlorophenyl)-2-(2- chlorophenyl)-1- methylpropyl]-(3,5- dichloro)benzenesulfonamide | 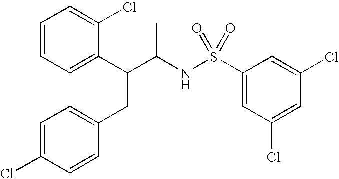 | 4.4 | 526 (M + 1) |
| 48. | N-[3-(4-chlorophenyl)-2-(6- chloroindol-N-yl)-1- methylpropyl]- benzenesulfonamide | 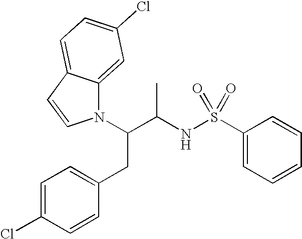 | 4.1 | 474 (M + 1) |
| 49. | N-[3-(4-chlorophenyl)-2-(5- chloroindol-N-yl)-1- methylpropyl]- benzenesulfonamide | 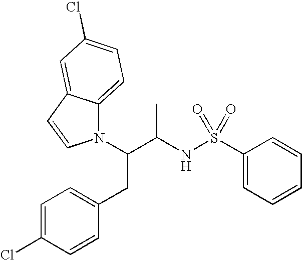 | 4.5 | 474 (M + 1) |
| 50. | N-[3-(4-chlorophenyl)-2- phenoxy-1-methylpropyl]- benzenesulfonamide | 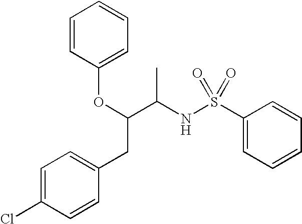 | 4.0 | 438 (M + Na) |
| 51. | N-[3-(4-chlorophenyl)-2-(2- chloro)phenoxy-1- methylpropyl]- benzenesulfonamide | 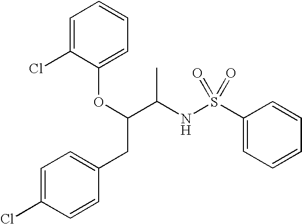 | 4.2 | 472 (M + Na) |
| 52. | N-[3-(4-chlorophenyl)-2-(4- chloro)phenoxy-1- methylpropyl]- benzenesulfonamide | 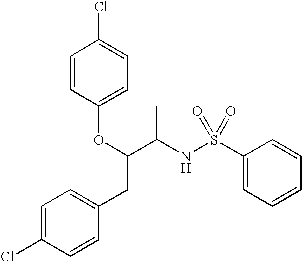 | 4.2 | 472 (M + Na) |
| 53. | N-[3-(4-chlorophenyl)-2-(4- bromo)phenoxy-1- methylpropyl]- benzenesulfonamide | 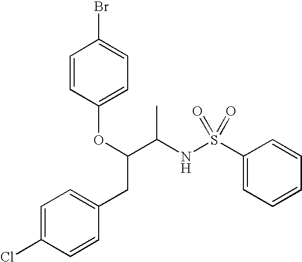 | 4.2 | 492, 494 (M + 1) |
| 54. | N-[3-(4-chlorophenyl)-2-(4- cyano)phenoxy-1- methylpropyl]- benzenesulfonamide | 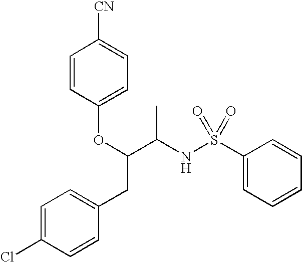 | 3.8 | 463 (M + Na) |
| 55. | N-[3-(4-chlorophenyl)-2-(4- chloro)phenoxy-1- methylpropyl]-(3,5- dichloro)benzenesulfonamide | 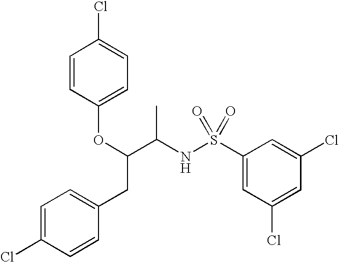 | 4.5 | 520 (M + 1) |
| 56. | N-[3-(4-chlorophenyl)-2-(4- chloro)phenoxy-1- methylpropyl]-(3- phenyoxy)benzenesulfonamide | 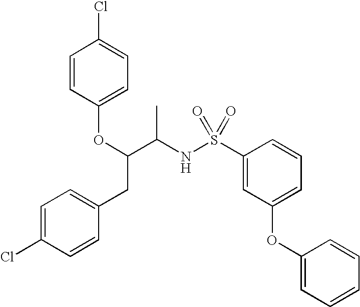 | 4.4 | 543 (M + 1) |
| 57. | N-[3-(4-chlorophenyl)-2-(4- chloro)phenoxy-1- methylpropyl]-biphenyl-3-yl- sulfonamide | 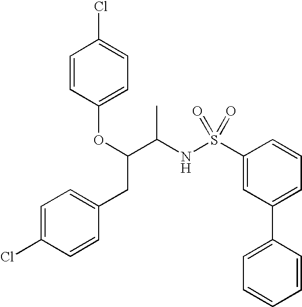 | 4.5 | 527 (M + 1) |

### EXAMPLE 58

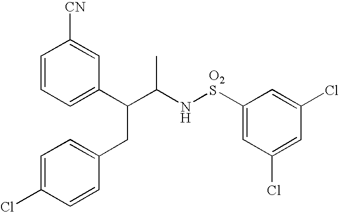

### N-[3-(4-chlorophenyl)-2(-3-cyanophenyl)-1-methylpropyl]-35-dichloro-benzenesulfonamide

To a solution of the N-[3-(4-chlorophenyl)-2-(3-cyanophenyl)-1-methylpropyl]amine hydrochloride (diastereomer α) (24 mg, 0.084 mmol) in dichloromethane (1 ml) at room temperature was added 3,5-dichlorophenylsulphonyl chloride (27 mg, 0.11 mmol) and diisopropylethylamine (29 μl, 0.169 mmol) and the mixture stirred at room temperature for 8 hours. The reaction mixture was purified without work-up by loading directly onto silica gel and eluting from 0-30% ethyl acetate/hexane to give the title racemic compound as a clear oil. ¹H NMR (500 MHz, CDCl₃): δ 7.68 (d, J=2.1 Hz, 2H), 7.58 (t, J=2.1 Hz, 1H), 7.54 (dt, J=7.6, 1.4 Hz, 1H), 7.40 (m, 3H), 7.20 (d, J=8.4 Hz, 2H), 6.93 (d, J=8.5 Hz, 2H), 4.55 (d, J=8.9 Hz, 1H), 3.59 (m, 1H), 3.19 (dd, J=13.9, 6.4 Hz, 1H), 3.11 (dt, J=9.1, 6.4 Hz, 1H), 2.85 (dd, J=14, 9.4 Hz, 1H) 0.92 (d, J=6.6 Hz, 3H). LC-MS: m/e 493 (M+H)⁺ (4.21 min). The enantiomers were separated by chiral HPLC on a Chiralcel OC 4.5 mm×250 mm column eluting with 10% ethanol/hexane at 8 ml/min to give enantiomer A and enantiomer B.

Examples 59-74 (Table 5) were prepared following the procedures described in Example 58 substituting with the appropriate sulfonyl chlorides.

TABLE 5

| | | | | HPLC-mass |
|---|---|---|---|---|
| Ex. | | | Retention | spectrum m/e |
| No. | Name | Structure | time (min) | (M + H) |
| 59. | N-[3-(4-chlorophenyl)-2- (3-cyanophenyl)-1- methylpropyl]- benzenesulfonamide | 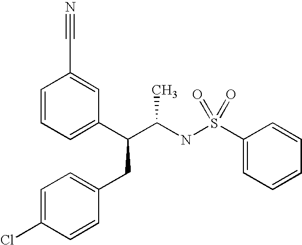 | 3.86 | 425 |
| 60. | N-[3-(4-chlorophenyl)-2 (3-cyanophenyl)-1- methylpropyl]-α- toluenesulfonamide | 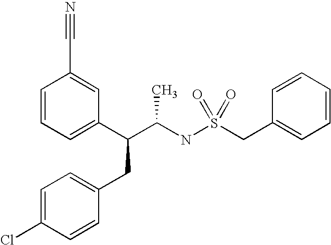 | 3.87 | 439 |
| 61. | N-[3-(4-chlorophenyl)-2- (3-cyanophenyl)-1- methylpropyl]-2- phenylethylsulfonamide | 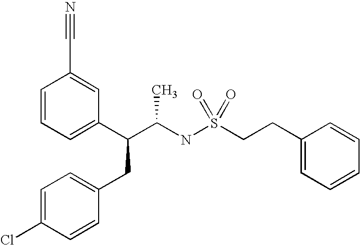 | 3.97 | 453 |
| 62. | N-[3-(4-chlorophenyl)-2- (3-cyanophenyl)-1- methylpropyl]-4-chloro- benzenesulfonamide | 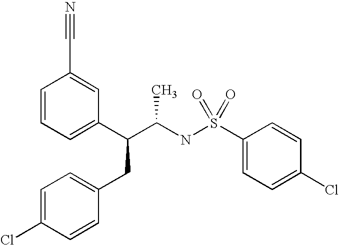 | 4.03 | 459 |
| 63. | N-[3-(4-chlorophenyl)-2- (3-cyanophenyl)-1- methylpropyl]-3-chloro- benzenesulfonamide | 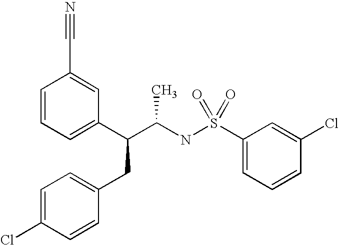 | 4.02 | 459 |
| 64. | N-[3-(4-chlorophenyl)-2- (3-cyanophenyl)-1- methylpropyl]-2-chloro- benzenesulfonamide | 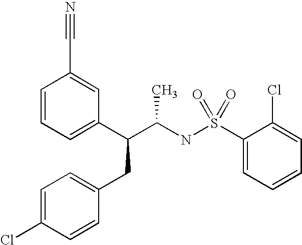 | 3.95 | 459 |
| 65. | N-[3-(4-chlorophenyl)-2- (3-cyanophenyl)-1- methylpropyl]-4- methoxy- benzenesulfonamide | 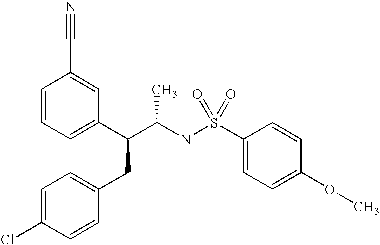 | 3.86 | 455 |
| 66. | N-[3-(4-chlorophenyl)-2- (3-cyanophenyl)-1- methylpropyl]- methanesulfonamide | 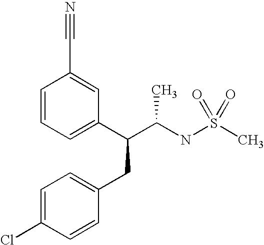 | 3.45 | 363 |
| 67. | N-[3-(4-chlorophenyl)-2- (3-cyanophenyl)-1- methylpropyl]-4-methyl- benzenesulfonamide | 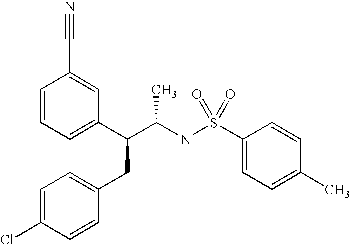 | 3.96 | 439 |
| 68. | N-[3-(4-chlorophenyl)-2- (3-cyanophenyl)-1- methylpropyl]-4- trifluoromethyl- benzenesulfonamide | 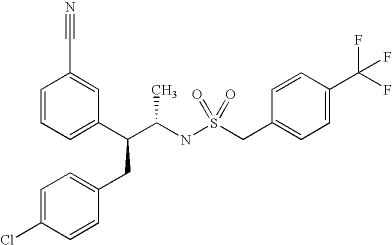 | 4.00 | 507 |
| 69. | N-[3-(4-chlorophenyl)-2- (3-cyanophenyl)-1- methylpropyl]-4-chloro- α-toluenesulfonamide | 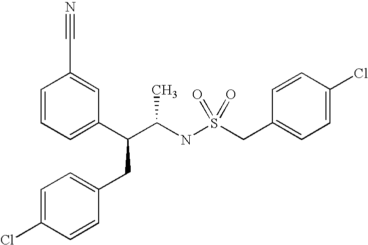 | 4.00 | 473 |
| 70. | N-[3-(4-chlorophenyl)-2- (3-cyanophenyl)-1- methylpropyl]-3- trifluoromethyl-α- toluenesulfonamide | 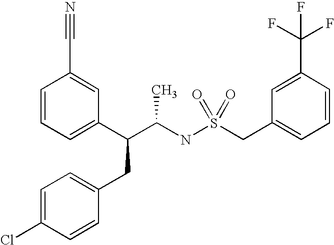 | 3.98 | 507 |
| 71. | N-[3-(4-chlorophenyl)-2- (3-cyanophenyl)-1- methylpropyl]-4-fluoro-α- toluenesulfonamide | 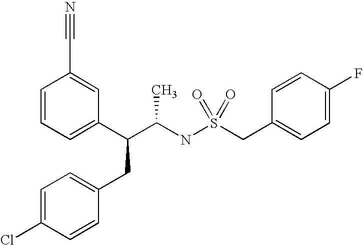 | 3.87 | 457 |
| 72. | N-[3-(4-chlorophenyl)-2- (3-cyanophenyl)-1- methylpropyl]-1,1- dimethyl- ethylsulfonamide | 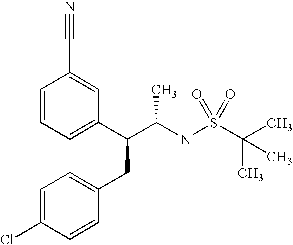 | 3.79 | 405 |
| 73. | N-[3-(4-chlorophenyl)-2- (3-cyanophenyl)-1- methylpropyl]-2- naphthylenesulfonamide | 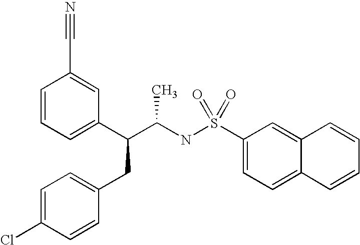 | 4.07 | 475 |
| 74. | N-[3-(4-chlorophenyl)-2- (3-cyanophenyl)-1- methylpropyl]-n- butylsulfonamide | 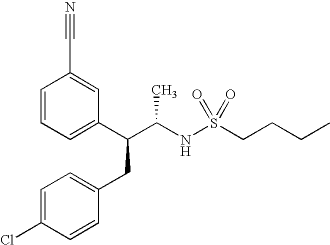 | 3.78 | 405 |

While the foregoing specification teaches the principles of the present invention, with examples provided for the purpose of illustration, it is understood that the practice of the invention encompasses all of the usual variations, adoptions, or modifications, as being within the scope of the following claims and their equivalents.

For example, effective dosages other than the particular dosages as set forth herein above may be applicable as a consequence of variations in the responsiveness of the mammal being treated for any of the indications for the compounds of the invention indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

### BIOLOGICAL EXAMPLE 1

Cannabinoid Receptor-1 (CB1) Binding Assau.

Binding affinity determination is based on recombinant human CB1 receptor expressed in Chinese Hamster Ovary (CHO) cells (Felder et al, Mol. Pharmacol. 48: 443-450, 1995). Total assay volume is 250 μL (240 μL CB1 receptor membrane solution plus 5 μL test compound solution plus 5 μL [3H]CP-55940 solution). Final concentration of [3H]CP-55940 is 0.6 nM. Binding buffer contains 50 mM Tris-HCl, pH7.4, 2.5 mM EDTA, 5 mM MgCl₂, 0.5 mg/mL fatty acid free bovine serum albumin and protease inhibitors (Cat#P8340, from Sigma). To initiate the binding reaction, 5 μL of radioligand solution is added, the mixture is incubated with gentle shaking on a shaker for 1.5 hours at 30° C. The binding is terminated by using 96-well harvester and filtering through GF/C filter presoaked in 0.05% polyethylenimine. The bound radiolabel is quantitated using scintillation counter. Apparent binding affinities for various compounds are calculated from IC₅₀ values (DeBlasi et al., Trends Pharmacol Sci 10: 227-229, 1989).

The binding assay for CB2 receptor is done similarly with recombinant human CB2 receptor expressed in CHO cells.

The compounds, found in Examples 1-74 and listed in Tables 1-5 were tested in the above assay and found to have an IC₅₀ value of 2 micromolar or less. Selective CB1 antagonist/inverse agonist compounds have IC₅₀S 100-fold greater in the CB2 binding assay than in the CB1 assay, and generally have IC50s of greater than one micromolar in the CB2 binding assay.

### BIOLOGICAL EXAMPLE 2

Cannabinoid Receptor-1 (CB1) Functional Activity Assay.

The functional activation of CB1 receptor is based on recombinant human CB1 receptor expressed in CHO cells (Felder et al, Mol. Pharmacol. 48: 443-450, 1995). To determine the agonist activity or inverse agonist activity of any test compound, 50 μL of CB1-CHO cell suspension are mixed with test compound and 70 μL assay buffer containing 0.34 mM 3-isobutyl-1-methylxanthine and 5.1 μM of forskolin in 96-well plates. The assay buffer is comprised of Earle's Balanced Salt Solution supplemented with 5 mM MgCl₂. 1 mM glutamine, 10 mM HEPES, and 1 mg/mL bovine serum albumin. The mixture is incubated at room temperature for 30 minutes, and terminated by adding 30 μl/well of 0.5M HCl. The total intracellular cAMP level is quantitated using the New England Nuclear Flashplate and cAMP radioimmunoassay kit.

To determine the antagonist activity of test compound, the reaction mixture also contains 0.5 nM of the agonist CP55940, and the reversal of the CP55940 effect is quantitated. Alternatively, a series of dose response curves for CP55940 is performed with increasing concentration of the test compound in each of the dose response curves.

The functional assay for the CB2 receptor is done similarly with recombinant human CB2 receptor expressed in CHO cells.

CB1 antagonist/inverse agonist compounds of the present invention generally have EC₅₀s of less than 1 micromolar in the CB1 functional assay and selective CB1 antagonist/inverse agonists have generally have EC50s of greater than 1 micromolar in the CB2 functional assay.

### BIOLOGICAL EXAMPLE 3

Acute Food Intake Studies in Rats or Mice: General Procedure

Adult rats or mice are used in these studies. After at least 2 days of acclimation to the vivarium conditions (controlled humidity and temperature, lights on for 12 hours out of 24 hours) food is removed from rodent cages. Experimental compounds or their vehicles are administered orally, intraperitoneally, subcutaneously or intravenously before the return of a known amount of food to cage. The optimal interval between dosing and food presentation is based on the half-life of the compound based on when brain concentrations of the compound is the highest. Food remaining is measured at several intervals. Food intake is calculated as grams of food eaten per gram of body weight within each time interval and the appetite-suppressant effect of the compounds are compared to the effect of vehicle. In these experiments many strains of mouse or rat, and several standard rodent chows can be used.

### BIOLOGICAL EXAMPLE 4

Chronic Weight Reduction Studies in Rats or Mice: General Procedure

Adult rats or mice are used in these studies. Upon or soon after weaning, rats or mice are made obese due to exclusive access to diets containing fat and sucrose in higher proportions than in the control diet. The rat strains commonly used include the Sprague Dawley bred through Charles River Laboratories. Although several mouse strains may be used, c57B1/6 mice are more prone to obesity and hyperinsulinemia than other strains. Common diets used to induce obesity include: Research Diets D12266B (32% fat) or D12451 (45% fat) and BioServ S3282 (60% fat). The rodents ingest chow until they are significantly heavier and have a higher proportion of body fat than control diet rats, often 9 weeks. The rodents receive injections (1 to 4 per day) or continuous infusions of experimental compounds or their vehicles either orally, intraperitoneally, subcutaneously or intravenously. Food intake and body weights are measured daily or more frequently. Food intake is calculated as grams of food eaten per gram of body weight within each time interval and the appetite-suppressant and weight loss effects of the compounds are compared to the effects of vehicle.

1. A compound of structural formula I: 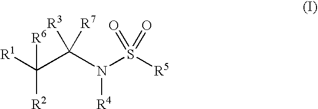
or a pharmaceutically acceptable salt or stereoisomer thereof, wherein:
R¹ is chosen from:
(1) C₁₋₁₀alkyl,
(2) C₃₋₁₀cycloalkyl-C₀₋₄alkyl,
(3) cycloheteroalkyl-C₀₋₄alkyl,
(4) aryl-C₀₋₄alkyl,
(5) heteroaryl-C₁₋₄alkyl,
(6) —OR^{d},
(7) —SR^{d},
(8) —(C═O)_{z}NR^{c}R^{d},
(9) —NR^{c}C(O)R^{d}, and
(10) —CO₂R^{d},
wherein each alkyl is optionally substituted with one to four substituents independently chosen from R^{a}, and each cycloalkyl, and cycloheteroalkyl, aryl, and heteroaryl are optionally substituted with one to four substituents independently chosen from R^{b};
R² is chosen from:
(1) C₁₋₁₀alkyl,
(2) C₃₋₁₀cycloalkyl-C₀₋₄akyl,
(3) cycloheteroalkyl-C₀₋₄alkyl,
(4) aryl-C₀₋₄alkyl, and
(5) heteroaryl-C₀₋₄alkyl,
wherein each alkyl is optionally substituted with one to four substituents independently chosen from R^{a}, and each cycloalkyl, cycloheteroalkyl, aryl and heteroaryl is optionally substituted with one to four substituents independently chosen from R^{b};
R³ and R⁷ are each independently chosen from:
(1) hydrogen,
(2) C₃₋₁₀cycloalkyl-C₀₋₄alkyl,
(3) cycloheteroalkyl-C₀₋₄alkyl,
(4) aryl-C₀₋₄alkyl,
(5) heteroaryl-C₀₋₄alkyl, and
(6) C₁₋₄alkyl,
wherein each alkyl is optionally substituted with one to four substituents independently chosen from R^{a}, and each cycloalkyl, cycloheteroalkyl, aryl and heteroaryl is optionally substituted with one to four substituents independently chosen from R^{b};
R⁴ is chosen from:
(1) hydrogen, and
(2) C₁₋₄alkyl,
wherein each alkyl is optionally substituted with one to four substituents independently chosen from R^{a};
R⁵ is chosen from:
(1) C₁₋₄alkyl,
(2) C₂₋₁₀alkenyl,
(3) C₂₋₁₀alkynyl,
(4) C₃₋₁₀cycloalkyl-C₀₋₄alkyl,
(5) cycloheteroalkyl-C₀₋₄alkyl,
(6) aryl-C₀₋₄alkyl,
(7) heteroaryl-C₁₋₄alkyl,
(8) —NR^{c}R^{d}, and
(9) —NR^{c}C(O)R^{d},
wherein alkyl, alkenyl, and alkynyl are optionally substituted with one to four substituents independently chosen from R^{a} and cycloalkyl, cycloheteroalkyl, aryl and heteroaryl are optionally substituted with one to four substituents independently chosen from R^{b};
R⁶ is chosen from:
(1) hydrogen,
(2) hydroxyl,
(3) C₁₋₄alkyl,
(4) halogen, and
(5) cyano,
provided that when R¹ is —OR^{d}, —SR^{d}, or —NR^{c}C(O)R^{d}, then R⁶ is chosen from hydrogen and C₁₋₄alkyl;
each R^{a} is independently chosen from:
(1) —OR^{d},
(2) —NR^{c}S(O)ₘR^{d},
(3) halogen,
(4) —SR^{d},
(5) —S(O)ₘNR^{c}R^{d},
(6) —(C═O)_{z}NR^{c}R^{d},
(7) —C(O)R^{d},
(8) —CO₂R^{d},
(9) —CN,
(10) —NR^{c}C(O)R^{d},
(11) —NR^{c}C(O)OR^{d},
(12) —NR^{c}C(O)NR^{c}R^{d},
(13) —CF₃,
(14) —OCF₃, and
(15) cycloheteroalkyl;
each R^{b} is independently chosen from:
(1) R^{a},
(2) C₁₋₁₀alkyl,
(3) oxo,
(4) arylC₀₋₄alkyl, and
(5) heteroarylC₀₋₄alkyl,
R^{c} and R^{d} are independently chosen from:
(1) hydrogen,
(2) C₁₋₁₀alkyl,
(3) C₂₋₁₀alkenyl,
(4) cycloalkyl-C₀₋₁₀alkyl;
(5) cycloheteroalkyl-C₀₋₁₀alkyl;
(6) aryl-C₀₋₁₀alkyl, and
(7) heteroaryl-C₁₋₁₀alkyl, wherein
R^{c} and R^{d} together with the atom(s) to which they are attached optionally form a heterocyclic ring of 4 to 7 members containing 0-2 additional heteroatoms independently chosen from oxygen, sulfur and N—R_{g}, and
each R^{c} and R^{d} can be optionally substituted with one to three substituents chosen from R^{h}; each R_{g} is independently chosen from
(1) C₁₋₁₀alkyl,
(2) —C(O)R^{c},
(3) —C(O)H,
(4) —C(O)C₁₋₁₀alkyl,
(5) —C(O)C₂₋₁₀alkenyl,
(6) —C(O)C₀₋₁₀alkylcycloalkyl,
(7) —C(O)C₀₋₁₀alkylcycloheteroalkyl,
(8) —C(O)C₀₋₁₀alkylaryl, and
(9) —C(O)C₀₋₁₀alkyl heteroaryl;
each R^{h} is independently chosen from:
(1) halogen,
(2) C₁₋₁₀alkyl,
(3) —O—C₁₋₄alkyl,
(4) —S—C₁₋₄alkyl,
(5) —CN,
(6) —NO₂,
(7) —CF₃, and
(8) —OCF₃;
m is chosen from 1 and 2; and
z is chosen from 0 and 1.

2. A compound according to claim 1, wherein R⁵ is chosen from: C₁₋₁₀alkyl, aryl-C₀₋₄alkyl, and heteroaryl-C₁₋₄alkyl, wherein alkyl is optionally substituted with one to four substituents independently chosen from R^{a} and aryl and heteroaryl are optionally substituted with one to four substituents independently chosen from R^{b}, and pharmaceutically acceptable salts thereof.

3. A compound according to claim 2, wherein R³ and R⁷ are each independently chosen from: hydrogen, aryl-C₀₋₄alkyl, and C₁₋₄alkyl, wherein each alkyl is optionally substituted with one to four substituents independently chosen from R^{a}, and each cycloalkyl, cycloheteroalkyl, aryl and heteroaryl is optionally substituted with one to four substituents independently chosen from R^{b}, and pharmaceutically acceptable salts thereof.

4. A compound according to claim 1 of structural formula I: 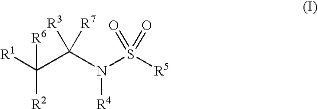
or a pharmaceutically acceptable salt or stereoisomer thereof, wherein:
R¹ is chosen from:
(1) C₁₋₁₀alkyl,
(2) C₃₋₁₀cycloalkyl-C₀₋₄alkyl,
(3) cycloheteroalkyl-C₀₋₄alkyl,
(4) aryl-C₀₋₄alkyl,
(5) heteroaryl-C₁₋₄alkyl,
(6) —OR^{d},
(7) —SR^{d},
(8) —(C═O)_{z}NR^{c}R^{d},
(9) —NR^{c}C(O)R^{d}, and
(10) —O₂R^{d},
wherein each alkyl is optionally substituted with one to four substituents independently chosen from R^{a}, and each cycloalkyl, and cycloheteroalkyl, aryl, and heteroaryl are optionally substituted with one to four substituents independently chosen from R^{b};
R² is R^{2′}—Y—;
Y is C₀₋₄alkyl optionally substituted with one to four substituents independently chosen from R^{a};
R^{2′} is chosen from: aryl and heteroaryl, wherein each aryl and heteroaryl is optionally substituted with one to four substituents independently chosen from R^{b};
R³ and R⁷ are each independently chosen from:
(1) hydrogen,
(2) aryl-C₀₋₄alkyl, and
(3) C₁₋₄alkyl,
wherein each alkyl is optionally substituted with one to four substituents independently chosen from R^{a}, and each cycloalkyl, cycloheteroalkyl, aryl and heteroaryl is optionally substituted with one to four substituents independently chosen from R^{b};
R⁴ is chosen from:
(1) hydrogen, and
(2) C₁₋₄alkyl,
wherein each alkyl is optionally substituted with one to four substituents independently chosen from R^{a};
R⁵ is chosen from:
(1) C₁₋₁₀alkyl,
(2) C₂₋₁₀alkenyl,
(3) C₂₋₁₀alkynyl,
(4) C₃₋₁₀cycloalkyl-C₀₋₄alkyl,
(5) cycloheteroalkyl-C₀₋₄alkyl,
(6) aryl-C₀₋₄alkyl,
(7) heteroaryl-C₁₋₄alkyl,
(8) —NR^{c}R^{d}, and
(9) —NR^{c}C(O)R^{d},
wherein alkyl, alkenyl, and alkynyl, are optionally substituted with one to four substituents independently chosen from R^{a} and cycloalkyl, cycloheteroalkyl, aryl and heteroaryl are optionally substituted with one to four substituents independently chosen from R^{b};
R⁶ is chosen from:
(1) hydrogen,
(2) hydroxyl,
(3) C₁₋₄alkyl,
(4) halogen, and
(5) cyano,
provided that when R¹ is —OR^{d}, —SR^{d}, or —NR^{c}C(O)R^{d}, then R⁶ is chosen from hydrogen and C₁₋₄alkyl;
each R^{a} is independently chosen from:
(1) —OR^{d},
(2) —NR^{c}S(O)ₘR^{d},
(3) halogen,
(4) —SR^{d},
(5) —S(O)ₘNR^{c}R^{d},
(6) —(C═O)_{z}NR^{c}R^{d},
(7) —C(O)R^{d},
(8) —CO₂R^{d},
(9) —CN,
(10) —NR^{c}C(O)R^{d},
(11) —NR^{c}C(O)OR^{d},
(12) —NR^{c}C(O)NR^{c}R^{d},
(13) —CF₃,
(14) —OCF₃, and
(15) cycloheteroalkyl;
each R^{b} is independently chosen from:
(1) R^{a},
(2) C₁₋₁₀alkyl,
(3) oxo,
(4) arylC₀₋₄alkyl, and
(5) heteroarylC₀₋₄alkyl;
R^{c} and R^{d} are independently chosen from:
(1) hydrogen,
(2) C₁₋₁₀alkyl,
(3) C₂₋₁₀alkenyl,
(4) cycloalkyl-C₀₋₁₀alkyl;
(5) cycloheteroalkyl-C₀₋₁₀alkyl;
(6) aryl-C₀₋₁₀alkyl, and
(7) heteroaryl-C₁₋₁₀alkyl, or
R^{c} and R^{d} together with the atom(s) to which they are attached form a heterocyclic ring of 4 to 7 members containing 0-2 additional heteroatoms independently chosen from oxygen, sulfur and N—R_{g},
each R^{c} and R^{d} may be unsubstituted or substituted with one to three substituents chosen from R^{h};
each R_{g} is independently chosen from
(1) C₁₋₁₀alkyl, and
(2) —C(O)R^{c};
each R^{h} is independently chosen from:
(1) halogen,
(2) C₁₋₁₀alkyl,
(3) —O—C₁₋₄alkyl,
(4) —S—C₁₋₄alkyl,
(5) —CN,
(6) —NO₂,
(7) —CF₃, and
(8) —OCF₃;
m is chosen from 1 and 2; and
z is chosen from 0 and 1.

5. A compound according to claim 4, wherein R^{2′} is chosen from: 2,3-dihydro-1H-indolyl, 3,4-dihydroquinolinyl, phenyl, benzyl, and pyridinyl, and R^{2′} is optionally substituted with one to four substituents independently chosen from R^{b}, and pharmaceutically acceptable salts thereof.

6. A compound according to claim 5, wherein Y is —CH₂—, and pharmaceutically acceptable salts thereof.

7. A compound according to claim 1, of structural formula II 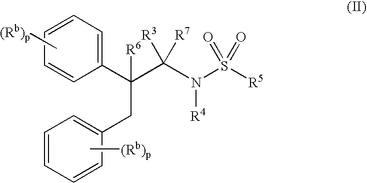
or a pharmaceutically acceptable salt or stereoisomer thereof, wherein:
R³ and R⁷ are each independently chosen from:
(1) hydrogen,
(2) C₃₋₁₀cycloalkyl-C₀₋₄alkyl,
(3) cycloheteroalkyl-C₀₋₄alkyl,
(4) aryl-C₀₋₄alkyl, and
(5) heteroaryl-C₀₋₄alkyl, and
(6) C₁₋₄alkyl,
wherein each alkyl is optionally substituted with one to four substituents independently chosen from R^{a}, and each cycloalkyl, cycloheteroalkyl, aryl and heteroaryl is optionally substituted with one to four substituents independently chosen from R^{b};
R⁴ is chosen from:
(1) hydrogen, and
(2) C₁₋₄alkyl,
wherein each alkyl is optionally substituted with one to four substituents independently chosen from R^{a};
R⁵ is chosen from:
(1) C₁₋₁₀alkyl,
(2) C₂₋₁₀alkenyl,
(3) C₂₋₁₀alkynyl,
(4) C₃₋₁₀cycloalkyl-C₀₋₄alkyl,
(5) cycloheteroalkyl-C₀₋₄alkyl,
(6) aryl-C₀₋₄alkyl,
(7) heteroaryl-C₁₋₄alkyl,
(8) —NR^{c}R^{d}, and
(9) —NR^{c}C(O)R^{d},
wherein alkyl, alkenyl, and alkynyl, are optionally substituted with one to four substituents independently chosen from R^{a} and cycloalkyl, cycloheteroalkyl, aryl and heteroaryl are optionally substituted with one to four substituents independently chosen from R^{b};
R⁶ is chosen from:
(1) hydrogen,
(2) hydroxyl,
(3) C₁₋₄alkyl,
(4) halogen, and
(5) cyano,
each R^{a} is independently chosen from:
(1) —OR^{d},
(2) —NR^{c}S(O)ₘR^{d},
(3) halogen,
(4) —SR^{d},
(5) —S(O)ₘNR^{c}R^{d},
(6) —C(═O)_{z}NR^{c}R^{d},
(7) —C(O)R^{d},
(8) —CO₂R_{d},
(9) —CN,]—NR^{c}C(O)R^{d},
(10) —NR^{c}C(O)OR^{d},
(11) —NR^{c}C(O)NR^{c}R^{d},
(12) —CF₃,
(13) —OCF₃, and
(14) cycloheteroalkyl;
each R^{b} is independently chosen from:
(1) R^{a},
(2) C₁₋₁₀alkyl,
(3) oxo,
(4) arylC₀₋₄alkyl, and
(5) heteroarylC₀₋₄alkyl,
R^{c} and R^{d} are independently chosen from:
(1) hydrogen,
(2) C₁₋₁₀alkyl,
(3) C₂₋₁₀alkenyl,
(4) cycloalkyl-C₀₋₁₀alkyl;
(5) cycloheteroalkyl-C₀₋₁₀alkyl;
(6) aryl-C₀₋₁₀alkyl, and
(7) heteroaryl-C₁₋₁₀alkyl, or
R^{c} and R^{d} together with the atom(s) to which they are attached form a heterocyclic ring of 4 to 7 members containing 0-2 additional heteroatoms independently chosen from oxygen, sulfur and N—R_{g},
each R^{c} and R^{d} may be unsubstituted or substituted with one to three substituents chosen from R^{h};
each R_{g} is independently chosen from
(1) C₁₋₁₀alkyl, and
(2) —C(O)R^{c};
each R^{h} is independently chosen from:
(1) halogen,
(2) C₁₋₁₀alkyl,
(3) —O—C₁₋₄alkyl,
(4) —S—C₁₋₄alkyl,
(5) —CN,
(6) —NO₂,
(7) —CF₃, and
(8) —OCF₃;
m is chosen from 1 and 2;
p is 0, 1, 2, 3, or 4; and
z is chosen from 0 and 1.

8. A compound according to claim 7, wherein:
R³ and R⁷ are each independently chosen from:
(1) hydrogen,
(2) aryl-C₀₋₄alkyl, and
(3) C₁₋₄alkyl,
wherein each alkyl is optionally substituted with one to four substituents independently chosen from R^{a}, and aryl is optionally substituted with one to four substituents independently chosen from R^{b}, and pharmaceutically acceptable salts thereof.

9. A compound according to claim 8, wherein R⁵ is chosen from: C₁₋₁₀alkyl, and aryl-C₀₋₄alkyl, wherein alkyl is optionally substituted with one to four substituents independently chosen from R^{a} and aryl is optionally substituted with one to four substituents independently chosen from R^{b}, and pharmaceutically acceptable salts thereof.

10. A compound according to claim 9, wherein R⁶ is chosen from hydrogen, hydroxyl, and halogen, or a pharmaceutically acceptable salt thereof.

11. A compound selected from the group consisting of:
N-[2-(4-chlorophenyl)-3-(2,4-dichlorophenyl)-1-methylpropyl]-2-methyl-2-propanesulfonamide,
N-[2-(4-chlorophenyl)-3-(4-chloro-2-fluorophenyl)-1-methylpropyl]-2-methyl-2-propanesulfonamide,
N-[2,3-bis(4-chlorophenyl)-1-methylpropyl]-2-methyl-2-propanesulfonamide,
N-[3-(4-chlorophenyl)-2-phenyl-1-methylpropyl]-2-methyl-2-propanesulfonamide,
N-[2,3-diphenyl-1-methylpropyl]-2-methyl-2-propanesulfonamide,
N-[2-(4-chlorophenyl)-3-phenyl-1-methylpropyl]-2-methyl-2-propanesulfonamide,
N-[2,3-diphenyl-1-methylpropyl]-2-methyl-2-propanesulfonamide,
N-[2,3-bis(4-chlorophenyl)-1-methylpropyl]-1,1-dimethylphenethylsulfonamide,
N-[3-(4-chlorophenyl)-2-phenyl-1-methylpropyl]-1,1-dimethylphenethylsulfonamide,
N-[2-(4-chlorophenyl)-3-phenyl-1-methylpropyl]-1,1-dimethylphenethylsulfonamide,
N-[2,3-diphenyl)-1-methylpropyl]-1,1-dimethylphenethylsulfonamide,
N-[2,3-bis(4-chlorophenyl)-1-methylpropyl]-1,1-dimethylphenethylsulfonamide,
N-[2-(4-chlorophenyl)-3-(2,4-dichlorophenyl)-1-methylpropyl]-1,1-dimethylphenethylsulfonamide,
N-[2,3-bis(4-chlorophenyl)-1-methylpropyl]-2-naphthalenesulfonamide,
N-[2-(4-chlorophenyl)-3-(2,4-dichlorophenyl)-1-methylpropyl]-2-naphthalenesulfonamide,
N-[2-(4-chlorophenyl)-2-phenyl-1-methylpropyl]-2-naphthalenesulfonamide,
N-[2,3-diphenyl-1-methylpropyl]-2-naphthalenesulfonamide,
N-[2-(3-bromophenyl)-3-(4-chlorophenyl)-2-hydroxyl-1(S)-methylpropyl]-4-nitrobenzenesulfonamide,
N-[2-(3-bromophenyl)-3-(4-chlorophenyl)-2-fluoro-1(S)-methylpropyl]-4-nitrobenzenesulfonamide,
N-[2,3-bis-(4-chlorophenyl)-1-methylpropyl]-4-fluorobenzenesulfonamide,
N-[2,3-bis-(4-chlorophenyl)-propyl]-benzenesulfonamide,
N-[2,3-bis-(4-chlorophenyl)-propyl]-4-chlorobenzenesulfonamide,
N-[2,3-bis-(4-chlorophenyl)-propyl]-3-chlorobenzenesulfonamide,
N-[3-(4-chlorophenyl)-1-methyl-2-phenylpropyl]-benzenesulfonamide,
N-[3-(4-chlorophenyl)-1-methyl-2-phenylpropyl]-4-chlorobenzenesulfonamide,
N-[3-(4-chlorophenyl)-1-methyl-2-phenylpropyl]-4-fluorobenzenesulfonamide,
N-[3-(4-chlorophenyl)-1-methyl-2-phenylpropyl]-3-chlorobenzenesulfonamide,
N-[2,3-bis-(4-chlorophenyl)-1-methylpropyl]-benzenesulfonamide,
N-[2,3-bis-(4-chlorophenyl)-1-methylpropyl]-4-chlorobenzenesulfonamide,
N-[2,3-bis-(4-chlorophenyl)-1-methylpropyl]-3-chlorobenzenesulfonamide,
N-[2,3-bis-(4-chlorophenyl)-propyl]-1-phenylmethanesulfonamide,
N-[2,3-bis-(4-chlorophenyl)-1-methylpropyl]-1-phenylmethanesulfonamide,
N-[3-4-chlorophenyl)-1-methyl-2-phenylpropyl]-1-phenylmethanesulfonamide,
N-[2,3-bis-(4-chlorophenyl)-1-methylpropyl]-3,4-dichlorobenzenesulfonamide,
N-[2,3-bis-(4-chlorophenyl)-1-methylpropyl]-3,5-dichlorobenzenesulfonamide,
N-[2,3-bis-(4-chlorophenyl)-1-methylpropyl]-2,3,4-trichlorobenzenesulfonamide,
N-[2,3-bis-(4-chlorophenyl)-propyl]-3,4-dichlorobenzenesulfonamide,
N-[2,3-bis-(4-chlorophenyl)-propyl]-3,5-dichlorobenzenesulfonamide,
N-[2,3-bis-(4-chlorophenyl)-propyl]-2,3,4-trichlorobenzenesulfonamide,
N-[3-(4-chlorophenyl)-2-(3-cyanophenyl)-1-methylpropyl]-3,5-dichloro-benzenesulfonamide,
N-[3-(4-chlorophenyl)-2-(3-cyanophenyl)-1-methylpropyl]-benzenesulfonamide,
N-[3-(4-chlorophenyl)-2-(3-cyanophenyl)-1-methylpropyl]-□-toluenesulfonamide,
N-[3-(4-chlorophenyl)-2-(3-cyanophenyl)-1-methylpropyl]-2-phenylethylsulfonamide,
N-[3-(4-chlorophenyl)-2-(3-cyanophenyl)-1-methylpropyl]-4-chloro-benzenesulfonamide,
N-[3-(4-chlorophenyl)-2-(3-cyanophenyl)-1-methylpropyl]-3-chloro-benzenesulfonamide,
N-[3-(4-chlorophenyl)-2-(3-cyanophenyl)-1-methylpropyl]-2-chloro-benzenesulfonamide,
N-[3-(4-chlorophenyl)-2-(3-cyanophenyl)-1-methylpropyl]-4-methoxy-benzenesulfonamide,
N-[3-(4-chlorophenyl)-2-(3-cyanophenyl)-1-methylpropyl]-4-methyl-benzenesulfonamide,
N-[3-(4-chlorophenyl)-2-(3-cyanophenyl)-1-methylpropyl]-4-methyl-benzenesulfonamide,
N-[3-(4-chlorophenyl)-2-(3-cyanophenyl)-1-methylpropyl]-4-trifluoromethyl-benzenesulfonamide,
N-[3-(4-chlorophenyl)-2-(3-cyanophenyl)-1-methylpropyl]-4-chloro-□-toluenesulfonamide,
N-[3-(4-chlorophenyl)-2-(3-cyanophenyl)-1-methylpropyl]-3-trifluoromethyl-□-toluenesulfonamide,
N-[3-(4-chlorophenyl)-2-(3-cyanophenyl)-1-methylpropyl]-4-fluoro-□-toluenesulfonamide,
N-[3-(4-chlorophenyl)-2-(3-cyanophenyl)-1-methylpropyl]-1,1-dimethyl-ethylsulfonamide,
N-[3-(4-chlorophenyl)-2-(3-cyanophenyl)-1-methylpropyl]-2-naphthylenesulfonamide,
N-[3-(4-chlorophenyl)-2-(3-bromophenyl)-1-methylpropyl]-benzenesulfonamide,
N-[3-(4-chlorophenyl)-2-(2-chlorophenyl)-1-methylpropyl]-benzenesulfonamide,
N-[3-(4-chlorophenyl)-2-(2-chlorophenyl)-1-methylpropyl]-(3,5-dichloro)benzenesulfonamide,
N-[3-(4-chlorophenyl)-2-(6-chloroindol-N-yl)-1-methylpropyl]-benzenesulfonamide,
N-[3-(4-chlorophenyl)-2-(5-chloroindol-N-yl)-1-methylpropyl]-benzenesulfonamide,
N-[3-(4-chlorophenyl)-2-phenoxy-1-methylpropyl]-benzenesulfonamide,
N-[3-(4-chlorophenyl)-2-(2-chloro)phenoxy-1-methylpropyl]-benzenesulfonamide,
N-[3-(4-chlorophenyl)-2-(4-chloro)phenoxy-1-methylpropyl]-benzenesulfonamide,
N-[3-(4-chlorophenyl)-2-(4-bromo)phenoxy-1-methylpropyl]-benzenesulfonamide,
N-[3-(4-chlorophenyl)-2-(4-cyano)phenoxy-1-methylpropyl]-benzenesulfonamide,
N-[3-(4-chlorophenyl)-2-(4-chloro)phenoxy-1-methylpropyl]-(3,5-dichloro)benzenesulfonamide,
N-[3-(4-chlorophenyl)-2-(4-chloro)phenoxy-1-methylpropyl]-(3-phenyoxy)benzenesulfonamide,
N-[3-(4-chlorophenyl)-2-(4-chloro)phenoxy-1-methylpropyl]-biphenyl-3-yl-sulfonamide,
N-[3-(4-chlorophenyl)-2-(3-cyanophenyl)-1-methylpropyl]-n-butylsulfonamide, and pharmaceutically acceptable salts and stereoisomers thereof.

12-15. (canceled)

16. A method of treating a disease mediated by the Cannabinoid-1 receptor comprising administration to a patient in need of such treatment a non-toxic, therapeutically effective amount of a compound according to claim 1.

17. The method according to claim 16, wherein the disease mediated by the Cannabinoid-1 receptor is selected from: psychosis, memory deficit, cognitive disorders, migraine, neuropathy, neuro-inflammatory disorders, cerebral vascular accidents, head trauma, anxiety disorders, stress, epilepsy, Parkinson's disease, schizophrenia, substance abuse disorders, relating to opiates, alcohol, marijuana, and nicotine; constipation, chronic intestinal pseudo-obstruction, cirrhosis of the liver, asthma, and obesity or other eating disorders associated with excessive food intake.

18. A method of treating obesity comprising administration to an obese subject of a non-toxic, therapeutically effective amount of a compound according to claim 1.

19. A method of preventing obesity in a subject at risk therefor comprising administration to the subject of 0.01 to 1000 mg of a compound of claim 1.

20. A pharmaceutical composition comprising a pharmaceutically acceptable carrier and a compound according to claim 1, or a pharmaceutically acceptable salt thereof.
